(19) United States
(12) Patent Application Publication
(10) Pub. No.: US 2006/0200191 A1
(43) Pub. Date: Sep. 7, 2006

(54) Method and apparatuses for treating an intravascular occlusion

(76) Inventor: Gholam-Reza Zadno-Azizi, Fremont, CA (US)

Correspondence Address: MEDTRONIC VASCULAR, INC.;IP LEGAL DEPARTMENT, 3576 UNOCAL PLACE, SANTA ROSA, CA, 95403, US

(21) Appl. No.: 11/416,365

(22) Filed: May 2, 2006

Related U.S. Application Data
(62) Division of application No. 11/159,777, filed on Jun. 23, 2005.
Application No. 11/159,777 is a continuation of application No. 10/035,389, filed on Dec. 28, 2001, now Pat. No. 6,958,059.
Application No. 10/035,389 is a continuation-in-part of application No. 09/537,471, filed on Mar. 24, 2000, now Pat. No. 6,454,741.
Application No. 09/537,471 is a continuation of application No. 09/049,857, filed on Mar. 27, 1998, now Pat. No. 6,135,991.
Application No. 09/049,857 is a continuation-in-part of application No. 08/813,807, filed on Mar. 6, 1997, now abandoned.
Application No. 09/049,857 is a continuation-in-part of application No. 09/049,712, filed on Mar. 27, 1998, now Pat. No. 6,544,276.
Application No. 09/049,712 is a continuation-in-part of application No. 08/975,723, filed on Nov. 20, 1997, now Pat. No. 6,050,972.
Application No. 08/975,723 is a continuation-in-part of application No. 08/812,139, filed on Mar. 6, 1997, now abandoned.
Application No. 08/812,139 is a continuation-in-part of application No. 08/650,464, filed on May 20, 1996, now abandoned.

(51) Int. Cl. A61M 29/00

(52) U.S. Cl. 606/200; 604/101.05

(57) ABSTRACT

Methods for an intravascular occlusion are provided. A guidewire having an occlusive device such as balloon or a filter at one end is advanced across the occlusion using a guide catheter, and the occlusive device is expanded distal to the occlusion to occlude the blood vessel. The guide catheter may also have an occlusive device to occlude the vessel proximal to the occlusion. In a treatment method for the carotid arteries, occlusive devices may be provided in the external carotid artery, in the internal carotid artery, and in the common carotid artery.

### CROSS-REFERENCE TO RELATED APPLICATIONS

This application is a divisional of U.S. application Ser. No. 11/159,777, filed Jun. 23, 2005, which is a continuation of U.S. application Ser. No. 10/035,389, filed Dec. 28, 2001, now U.S. Pat. No. 6,958,059, which is a continuation-in-part of: U.S. application Ser. No. 09/537,471, filed Mar. 24, 2000, now U.S. Pat. No. 6,454,741, which is a continuation of U.S. application Ser. No. 09/049,857, filed Mar. 27, 1998, now U.S. Pat. No. 6,135,991, which is a continuation-in-part of U.S. application Ser. No. 08/813,807, filed Mar. 6, 1997, now abandoned; U.S. application Ser. No. 09/049,712, filed Mar. 27, 1998, now U.S. Pat. No. 6,544,276, which is a continuation-in-part of U.S. application Ser. No. 08/975,723, Nov. 20, 1997, now U.S. Pat. No. 6,050,972, which is a continuation-in-part of U.S. application Ser. No. 08/812,139, filed Mar. 6, 1997, abandoned, which is a continuation-in-part of U.S. application Ser. No. 08/650,464, filed May 20, 1996, now abandoned; U.S. application Ser. No. 09/438,030, filed Nov. 10, 1999, now U.S. Pat. No. 6,652,480; U.S. application Ser. No. 09/270,150, filed Mar. 16, 1999, now abandoned; U.S. application Ser. No. 09/837,872, filed Apr. 17, 2001, now abandoned, which is a continuation of U.S. application Ser. No. 09/415,607, filed Oct. 8, 1999, now U.S. Pat. No. 6,217,567, which is a continuation of U.S. application Ser. No. 08/812,876, filed Mar. 6, 1997, now U.S. Pat. No. 6,068,623; and U.S. application Ser. No. 09/314,054, filed May 18, 1999, now abandoned, which is a continuation of U.S. application Ser. No. 08/812,570, filed Mar. 6, 1997, now U.S. Pat. No. 6,022,336, which is a continuation-in-part of U.S. application Ser. No. 08/650,464, filed May 20, 1996, now abandoned; all of which are incorporated by reference in their entireties.

### BACKGROUND OF THE INVENTION

1. Field of the Invention

Certain embodiments disclosed relate to treating an intravascular occlusion. The methods are particularly well suited for treating stenoses or occlusions within saphenous vein grafts, coronary arteries, cerebral arteries and similar vessels.

2. Description of the Related Art

Human blood vessels often become occluded or completely blocked by plaque, thrombi, emboli or other substances, which reduces the blood carrying capacity of the vessel. Should the blockage occur at a critical location in the circulation, serious and permanent injury, or death, can occur. To prevent this, some form of medical intervention is usually performed when significant occlusion is detected, such as during an acute myocardial infarction (AMD.

Coronary heart disease is the leading cause of death in the United States and a common occurrence worldwide. Damage to or malfunction of the heart is caused by narrowing or blockage of the coronary arteries (atherosclerosis) that supply blood to the heart. The coronary arteries are first narrowed and may eventually be completely blocked by plaque, and may further be complicated by the formation of thrombi (blood clots) on the roughened surfaces of the plaques. AMI can result from atherosclerosis, especially from an occlusive or near occlusive thrombus overlying or adjacent to the atherosclerotic plaque, leading to death of portions of the heart muscle. Thrombi and emboli also often result from myocardial infarction, and these clots can block the coronary arteries, or can migrate further downstream, causing additional complications.

The carotid arteries are the main vessels which supply blood to the brain and face. The common carotid artery leads upwards from the aortic arch, branching into the internal carotid artery which feeds the brain, and the external carotid artery which feeds the head and face. The carotid arteries are first narrowed and may eventually be almost completely blocked by plaque, and may further be complicated by the formation of thrombi (blood clots) on the roughened surfaces of the plaques. Narrowing or blockage of the carotid arteries is often untreatable and can result in devastating physical and cognitive debilitation, and even death.

Various types of intervention techniques have been developed which facilitate the reduction or removal of the blockage in the blood vessel, allowing increased blood flow through the vessel. One technique for treating stenosis or occlusion of a blood vessel is balloon angioplasty. A balloon catheter is inserted into the narrowed or blocked area, and the balloon is inflated to expand the constricted area. In many cases, near normal blood flow is restored. It can be difficult, however, to treat plaque deposits and thrombi in the coronary arteries, because the coronary arteries are small, which makes accessing them with commonly used catheters difficult. Other types of intervention include atherectomy, deployment of stents, introduction of specific medication by infusion, and bypass surgery.

Furthermore, the fear of dislodging an embolus from an ulcerative plaque and the severe resulting consequences has prevented the widespread use of angioplasty in the carotid arteries. Because of the potential complications, the options for minimally invasive treatment of the carotid arteries are severely limited.

Carotid endarterectomy is another type of intervention for removal of blockages from the carotid arteries. In endarterectomy, the carotid bifurcation is exposed through an incision in the neck of the patient. Clamps are placed on either side of the occlusion to isolate it, and an incision made to open the artery. The occlusion is removed, the isolated area irrigated and aspirated, and the artery sutured closed. The clamps are removed to reestablish blood flow through the artery. In carotid endarterectomy, the emboli and debris are contained and directed by activating and deactivating the clamps. For example, after the clamps are in place, one on the common carotid artery and one on the internal carotid artery, the particles are contained between the two clamps. After the occlusion is removed, the clamp on the common carotid artery is opened, allowing blood to flow into the previously isolated area toward the clamp on the internal carotid. This blood flow is then aspirated through an external aspiration tube. The common carotid artery is then reclamped, and the clamp on the internal carotid opened. This causes blood to flow into the previously isolated area toward the clamp on the common carotid artery. The flow is then aspirated. The clamp on the internal carotid artery is closed, and the artery is sutured closed. This method allows for the flushing of debris into the area where aspiration occurs.

Alternatively, this method of clamping and unclamping the carotid arteries can be done after the incision in the artery is sutured closed. Using this method, it is hoped that any particles in the internal carotid artery will be forced back to the common carotid artery, then into the external carotid area, where serious complications are unlikely to arise from emboli.

Carotid endarterectomy is not without the serious risk of embolization and stroke caused by particles of the blocking material and other debris moving downstream to the brain, however.

There is therefore a need for improved methods of treatment of occluded vessels which decrease the risks to the patient.

### SUMMARY OF THE INVENTION

In one embodiment of the present invention, a method is provided for treating an intravascular occlusion. The method comprises delivering fluid containing an occlusion-treating drug at a location proximal to an intravascular occlusive device. The occlusive device may be a balloon, while the drug may be a thrombolytic agent, an anticoagulant or a radioisotope. The occlusive device is preferably delivered on a guidewire, with the occlusive device being actuated once the device is delivered distal to the occlusion. The drug is preferably delivered at a rate of between about 0.1 and 10 cc/second. In one embodiment, the drug travels proximally to distally, and once the drug or at least a portion thereof contacts the device, the drug or portion thereof travels in a distal to proximal direction, i.e., against the flow of blood. Correspondingly, because blood is flowing proximally to distally in the vessel, the blood flow localizes the drug at a desired treatment site in order to treat the occlusion.

The fluid-containing drug is preferably delivered through a catheter riding over the guidewire. In one embodiment, the catheter is an aspiration catheter. This allows the same lumen used for delivering drugs to aspirate any particles broken off by the drug treatment. Because the occlusive device is preferably actuated continuously during both drug delivery and aspiration, by delivering drugs and aspirating through the same catheter, the time that the occlusive device remains inflated is minimized.

In another embodiment of the present invention, a method for treating an intravascular occlusion comprises delivering an occlusive device at its distal end into a blood vessel to a site near said occlusion. A catheter having a proximal end and distal end is delivered to the site of said occlusion such that the distal end of the catheter is proximal to the occlusive device. The occlusive device on the guidewire is actuated at a location distal to said occlusion to at least partially occlude blood flow through the vessel. A drug-containing fluid is delivered from the distal end of the catheter such that at least a portion of the drug-containing fluid contacts the occlusive device.

In another embodiment of the present invention, a method of treating an intravascular occlusion in a blood vessel comprises delivering a guidewire having an occlusive device to the site of the occlusion such that the occlusive device is distal to the occlusion. A catheter is delivered having a proximal end and a distal end and a lumen extending therethrough to the site of the occlusion such that the distal end of the catheter is proximal to the occlusive device. The occlusive device is actuated to at least partially obstruct blood flow through the blood vessel. A treatment fluid is delivered through the lumen of the catheter such that the fluid flows in a proximal to distal direction out of the distal end of the catheter, and then flows in a distal to proximal direction after contacting the occlusive device. Particles generated by the action of the treatment fluid on the occlusion are aspirated through the lumen of the catheter at the distal end.

In another embodiment of the present invention, a method for crossing an intravascular occlusion in a blood vessel is provided. The method comprises delivering a hollow wire in a proximal to distal direction past the occlusion, and delivering fluids through a lumen in said hollow wire to dissolve the occlusion while crossing of the occlusion with the hollow wire.

In another embodiment of the present invention, a method for treating an intravascular occlusion, comprises delivering a catheter having a proximal end and a distal end and a lumen extending therethrough into a blood vessel to a site near said occlusion. The catheter has an occlusive device on the distal end. The occlusive device is actuated at a location distal to the occlusion to at least partially occlude blood flow through said vessel. A drug-containing fluid is injected through the lumen of the catheter across said occlusion in a distal to proximal direction. In one embodiment, the drug-containing fluid is delivered through a plurality of holes in the catheter proximal to the occlusive device. In another embodiment, the drug-containing fluid is delivered through a plurality of holes in a proximal face of an occlusive balloon.

In another embodiment, it is an objection of the present invention to provide an apparatus or an assembly and method which can be used with approved diagnostic and therapeutic devices while minimizing the opportunities for emboli to migrate downstream.

Another object of the present invention is to provide an apparatus or assembly and method of the above character which makes it possible to perform therapeutic procedures without using perfusion.

Another object of the invention is to provide an apparatus or assembly and method of the above character in which the proximal balloon utilized is a balloon carried by a guide wire.

Another object of the invention is to provide an apparatus or assembly and method of the above characters in which the inflation fitting carried by the proximal extremity of the balloon-on-a-wire is removable so that catheters can be slid over the wire without removal of the wire from the site in which it is disposed.

Another object of the present invention is to provide an apparatus or assembly and method for treating occluded vessels of the above character which makes it possible to prevent downstream flow of debris or emboli.

Another object of the invention is to provide an apparatus and method which makes it possible to reverse the flow of blood in an occluded vessel during the time that a stenosis is being crossed.

Another object of the invention is to provide an apparatus and method of the above character in which a negative pressure is created within the vessel to reverse the flow of blood in the vessel.

Another object of the invention is to provide an apparatus and method of the above character in which it is only necessary to stop the flow of blood in a vessel of a patient for a very short period of time.

Another object of the invention is to provide an apparatus and method in which a working space is provided in the vessel free of blood for treatment of the stenosis.

Another object of the invention is to provide an apparatus and method of the above character in which material which is dislodged during the treatment of the occlusion or stenosis is removed by suction.

Another object of the invention is to provide an apparatus and method of the above character in which blood is shunted around the working space.

Another object of the invention is to provide an apparatus and method in which a cutting device is utilized for treatment of the stenosis or atheroma in the vessel and in which the material removed from the stenosis or atheroma is aspirated out of the operating space.

Another object of the invention is to provide an apparatus and method of the above character in which the amount of material removed from the stenosis or atheroma can be precisely controlled.

Another object of the invention is to provide an apparatus and method of the above character which makes it possible to treat stenoses or occlusions in the vessel wnich are normally not accessible for surgical procedures.

Another object of the invention is to provide an apparatus and method of the above character which utilizes two spaced apart balloons to create the working space in the vessel.

Another object of the invention is to provide an apparatus and method of the above character that can be utilized to create a working space in a vessel having a bifurcation therein and in which the working space includes the bifurcation.

Another object of the invention is to provide an apparatus and method of the above character which utilizes three spaced apart balloons to create the working space in the vessel having a bifurcation therein.

Another object of the invention is to provide an apparatus and method of the above character which includes a control console for controlling the inflation of the blood flow pump.

Another object of the invention is to provide an apparatus and method of the above character which is particularly adapted for use with the carotid vessels.

Another aspect of the present invention is that the catheter system itself is provided with occlusive devices to form an emboli containment chamber. It will be noted that at least two such occlusive devices are needed to form a chamber in a straight vessel, while multiple occlusive devices may be necessary to provide emboli containment in the case of a branching vessel. Again, in this context, the term “occlusive device” makes reference to the blocking or containment of emboli within the chamber, since perfusion systems which provide occlusion to the emboli are within the scope of the present invention. Thus, various types of occlusive devices such as filters or expandable braids that allow particles of less than 20 micrometers to pass through while preventing the passage of larger particles, and including inflatable or expendable balloons such as those which are employed by the present catheter system or otherwise, are within the scope of the present invention. In one preferred embodiment, the outer catheter comprises a main catheter having an occlusive balloon mounted on the outer diameter thereof. The occlusive balloon is inflated by means of an inflation lumen formed in a wall of the main catheter. The inner catheter comprises what may be referred to as a guidewire, but which is also hollow to provide an inflation lumen for a second occlusive balloon mounted at the distal section thereof. This occlusive balloon remains inflated until the guide catheter crosses the site of the lesion Within the vessel. Thus, when inflated, these two occlusion balloons form an emboli containment chamber. The inner catheter provides a guidewire for those types of therapy devices which are in common use. One such catheter for a dedicated irrigation/aspiration catheter is positioned over the guidewire to form one of the irrigation/aspiration paths therewith.

In another embodiment, the present invention provides a novel method for containing and removing substances such as emboli from blood vessels. The method is particularly useful in bifurcated vessels, such as the carotid arteries and in other blood vessels above the aortic arch. In one embodiment of the method, there is provided at least one occlusive device such as a balloon or filter, a therapy catheter to treat the occlusion, and a source of aspiration to remove the debris created by the therapy. By utilizing the fluid pressure and flow within the blood vessel, this method can eliminate the need for a separate irrigation catheter and irrigation fluid. Alternatively, irrigation fluid may be provided to flush the area. The minimally invasive treatment allows occlusions to be treated more rapidly and less invasively than known methods, with reduced cost and risk to the patient.

In accordance with one aspect of the present invention, there is provided a method for the treatment of an occlusion in a carotid artery. A main catheter having a first occlusive device on its distal end is inserted into the artery, until the occlusive device is proximal to the occlusion. The first occlusive device is activated to occlude the artery proximal to the occlusion. An inner catheter having a second occlusive device on its distal end is inserted into the artery across the occlusion, until the occlusive device is distal to the occlusion. The second occlusive device is then activated to occlude the artery distal to the occlusion and create a working area surrounding the occlusion. By occlusive device is meant any device which is capable of preventing at least some particles or other debris from migrating downstream. Examples of occlusive devices include inflatable balloons, filters or braids, or other mechanical devices.

According to the foregoing aspect of the invention, a therapy catheter is then inserted into the working area and used to treat the occlusion. Appropriate treatment can include direct drug delivery to the site of the occlusion, angioplasty, cutting, scraping or pulverizing the occlusion, ablating the occlusion using ultrasound or a laser, deploying a stent within the artery, use of a thrombectomy or rheolitic device, or other treatments. Following treatment of the occlusion, the therapy catheter is removed. An aspiration catheter is then delivered to the working area, and the first occlusive device is deactivated to allow blood flow into the working area. Blood flow from collateral vessels prevent the movement of particles and debris downstream where they could cause serious complications. The blood flow also acts as irrigation fluid to create turbulence within the area. Aspiration of the working area is then performed to removed particles and debris. Aspiration can occur simultaneously with the deactivating of the first occlusive device, if desired. Alternatively, either step can be performed first.

In another aspect of the method of the present invention, the occlusive devices are activated and deactivated more than once. After the first occlusive device is deactivated to allow blood flow into the area, the occlusive device is reactivated. The second occlusive device is then deactivated, to allow blood flow in from the distal end of the working area. The second occlusive device is reactivated, and these steps can be repeated any number of times until sufficient irrigation and aspiration of the working area occurs.

In yet another aspect of the method, the first inner catheter with its occlusive device is delivered into one branch of a bifurcated vessel (such as the carotid artery), while a second inner catheter having a third occlusive device on its distal end is delivered into the other branch of the bifurcated vessel to occlude it. Aspiration then occurs in both branches of the artery to remove particles and debris.

In a further aspect of the method, aspiration occurs through the main catheter, and a separate aspiration catheter is not required. Following removal of the therapy catheter, and deactivation of the first occlusive device to allow blood flow into the working area, aspiration occurs through the distal end of the main catheter. This eliminates the need to deliver a separate aspiration catheter, thus saving time which is critical in these types of procedures.

If desired, an irrigation catheter can be delivered into the working area following the removal of the therapy catheter. The irrigation catheter is used to deliver irrigation fluid to the working area. Aspiration then occurs through the distal end of the main catheter. In this case, anatomical irrigation (the use of the patient's own blood flow for irrigation) as described above, is not used.

Yet another aspect of the method may be performed with a single occlusive device. A main catheter or guide catheter is first delivered into the carotid artery, with the distal end positioned just proximal to the occlusion. An inner catheter having an occlusive device on its distal end is then positioned with the occlusive device distal to the occlusion. The occlusive device is activated to occlude the artery distal to the occlusion. A therapy catheter is delivered into the artery until it reaches the occlusion and therapy is performed to reduce or eliminate the occlusion. The therapy catheter is removed, and an intermediate catheter is delivered to a position proximal to the occlusive device. Preferably, the distance between the proximal end of the occlusive device and the distal end of the intermediate catheter is narrowed at one point during aspiration to a distance of about 2 centimeters or less. The area just proximal to the occlusive device is aspirated, using the intermediate catheter, and then irrigated. The aspirating and irrigating steps can be repeated as often as necessary to facilitate the removal of particles and debris.

In another embodiment, the intermediate catheter has two or more lumens, such that aspiration and irrigation occur through different lumens within the same catheter. This prevents the possibility that aspirated particles will be flushed back into the patient when irrigation is begun.

In further aspects of the present invention, two and even three occlusive devices are employed. In the case of two occlusive devices, a main or guide catheter with an occlusive device on its distal end is delivered to the common carotid artery and the occlusive device is activated. Next, an inner catheter with an occlusive device is delivered distal to the occlusion in the internal carotid artery and activated, thus isolating the occlusion between the two occlusive devices. Therapy is performed on the occlusion, followed by aspiration, and irrigation if desired.

When three occlusive devices are used, an occlusive device is activated in the common carotid artery. An inner catheter with an occlusive device is then delivered to the external carotid artery and the occlusive device activated. Next, a second inner catheter is delivered to the internal carotid artery past the site of the occlusion and the occlusive device activated to occlude the internal carotid artery. Alternatively, the first inner catheter and occlusive device is delivered to the internal carotid artery and activated, followed by delivery and activation of the second inner catheter and occlusive device in the external carotid artery. In either case, the occlusion is completely isolated between the three occlusive devices. This is followed by therapy on the occlusion and sequential aspiration and irrigation as desired.

Accordingly, a carotid artery can be treated quickly and efficiently. The patient's own blood can serve as irrigation fluid, thereby eliminating the need for a separate irrigation catheter and supply of irrigation fluid. The working area may be cleaned in an efficient manner by performing repeated activation and deactivation of the occlusive devices surrounding the working area. The catheter-based approach reduces the amount of time required to complete the procedure, and allows normal blood flow in the vessel to be restored in a very short period of time. Use of a minimally invasive procedure reduces risks and trauma to the patient, decreases costs, and improves recovery time.

Another aspect of the invention comprises a method for the treatment of an occlusion in a branch of a bifurcated blood vessel having a common portion and two branches, such as the carotid artery, comprising providing an elongate member having an occlusive device at a distal end portion thereof, delivering the elongate member through the common portion of the bifurcated vessel and into a branch of the bifurcated vessel (such as the internal carotid artery), and positioning the occlusive device in said branch distal of the occlusion. The method further comprises sliding a therapy catheter on the elongate member, occluding said branch only on the distal side of the occlusion by actuating the occlusive device, treating the occlusion with the therapy catheter, and providing a second catheter having a fluid flow lumen in fluid communication with a fluid flow opening at a distal end portion of the second catheter. The method additionally comprises using the occlusive device to occlude said branch of the vessel while: (a) positioning the fluid flow opening of the second catheter in said branch of the vessel at a location between the occlusive device and the treated occlusion; and (b) applying fluid pressure to the fluid flow lumen to cause fluid flow along said branch, between (i) an intersection of said branch with the common portion and (ii) said location, whereby fluid flows across the treated occlusion; and then deactuating the occlusive device.

Still another aspect of the invention comprises a method for the treatment of an occlusion in a branch of a bifurcated blood vessel having a common portion and two branches, such as the carotid artery, comprising providing an elongate member having an occlusive device at a distal end portion thereof, delivering the elongate member through the common portion of the bifurcated vessel and into a branch of the bifurcated vessel (such as the internal carotid artery), positioning the occlusive device in said branch distal of the occlusion, sliding a therapy catheter on the elongate member, and occluding said branch on the distal side of the occlusion by actuating the occlusive device. The method further comprises treating the occlusion with the therapy catheter, removing the therapy catheter from said branch of the vessel, providing a second catheter having a fluid flow lumen in fluid communication with a fluid flow opening at a distal end portion of the second catheter, and sliding the second catheter on the elongate member after the removal of the therapy catheter. The method additionally comprises using the occlusive device to occlude said branch of the vessel while (a) positioning the fluid flow opening of the second catheter in said branch of the vessel at a location between the occlusive device and the treated occlusion; (b) applying fluid pressure to the fluid flow lumen to cause fluid flow along said branch, between (i) an intersection of said branch with the common portion and (ii) said location, whereby fluid flows across the treated occlusion; and then deactuating the occlusive device.

Yet another aspect of the invention comprises a method for the treatment of an occlusion in a branch of a bifurcated blood vessel having a common portion and two branches, such as the carotid artery, comprising providing an elongate member having an occlusive device at a distal end portion thereof, delivering the elongate member through the common portion of the bifurcated vessel and into a branch of the bifurcated vessel (such as the internal carotid artery), positioning the occlusive device in said branch distal of the occlusion, sliding a therapy catheter on the elongate member, occluding said branch only on the distal side of the occlusion by actuating the occlusive device, and treating the occlusion with the therapy catheter. The method further comprises using the occlusive device to occlude the branch of the vessel while: (a) delivering irrigation fluid to a distal end portion of the therapy catheter through an annulus between the therapy catheter and the elongate member; (b) passing the irrigation fluid out of a fluid flow opening in the distal end portion of the therapy catheter; and (c) positioning the fluid flow opening of the therapy catheter in said branch of the vessel at a location near the occlusive device between the occlusive device and the treated occlusion, such that fluid flows across the treated occlusion; and then deactuating the occlusive device.

Still another aspect of the invention comprises a method for the treatment of an occlusion in a branch of bifurcated blood vessel having a common portion and two branches, such as the carotid artery, comprising providing an elongate member having an occlusive device at a distal end portion thereof, delivering the elongate member through the common portion of the bifurcated vessel and into a branch of the bifurcated vessel (such as the internal carotid artery), positioning the occlusive device in said branch distal of the occlusion, positioning an outer catheter so that a portion of the outer catheter is in the common portion of the vessel, sliding a therapy catheter within the outer catheter and on the elongate member, actuating the occlusive device such that it occludes said branch of the vessel, and treating the occlusion with the therapy catheter. The method further comprises using the occlusive device to occlude the branch of the vessel while (a) delivering irrigation fluid to a distal end portion of the outer catheter; (b) passing the irrigation fluid out of a fluid flow opening in the distal end portion of the outer catheter; (c) positioning the fluid flow opening of the outer catheter in said branch of the vessel at a location between the occlusive device and the treated occlusion, such that fluid flows across the treated occlusion; and then deactuating the occlusive device.

Still another aspect of the invention comprises a method for treatment of an occlusion in a branch of a bifurcated blood vessel having a common portion and two branches, comprising positioning an occlusive device distal of the occlusion to occlude said branch of the vessel, treating the occlusion using a therapy device, delivering irrigation fluid between the occlusion and the occlusive device such that irrigation fluid flows across the treated occlusion towards an intersection of said branch and the common portion, wherein emboli in said branch are carried to the intersection, and allowing anatomical blood flow in the common portion to carry the emboli through another of the branches.

Yet another aspect of the invention comprises a method for the treatment of an occlusion in a blood vessel, such as the carotid artery, comprising providing an inner catheter comprising an elongate member having an occlusive device at a distal end portion thereof, delivering the elongate member through the vessel, positioning the occlusive device distal of the occlusion, sliding a therapy catheter on the elongate member, actuating the occlusive device such that it occludes the vessel, and treating the occlusion with the therapy catheter. The method further comprises uses the occlusive device to occlude the vessel while: (a) delivering irrigation fluid through the elongate member; (b) passing the irrigation fluid out of a fluid flow opening in the occlusive device such that fluid flows across the treated occlusion; and then deactuating the occlusive device. Still another aspect of the invention comprises a method of performing a medical procedure in a blood vessel using an expandable member which seals against walls of the blood vessel in response to application of an expansion force through a range of vessel diameters up to a maximum diameter beyond which sealing will not occur in the vessel, in which the method comprises positioning the expandable member in a selected blood vessel distal to an occlusion to be treated at a location where the vessel diameter is at least 20% less than said maximum diameter, applying an expansion force to cause the expandable member to expand into sealing contact with walls of the selected vessel at said location, and treating the occlusion while the expandable member is expanded, whereby the expandable member seals against walls of the selected vessel even if the diameter of the selected vessel at said location increases to said maximum diameter as a result of the treatment.

Another aspect of the invention comprises a method of treating an occlusion in a blood vessel, comprising positioning an expandable member distal to the occlusion to be treated, performing therapy on the occlusion, and using the expandable member to block migration of emboli created as a result of the therapy, while allowing blood to flow from one side to another side of the expandable member in a proximal to distal direction. The method further comprises positioning a fluid port of a catheter between the treated occlusion and the expandable member, and applying suction to the fluid port to aspirate fluid into the catheter while the fluid port is positioned between the treated occlusion and the expandable member.

Still another aspect of the invention comprises a method of treating an occlusion in a blood vessel, comprising positioning an expandable member distal to the occlusion to be treated, performing therapy on the occlusion, and using the expandable member to block migration of emboli created as a result of the therapy, while allowing blood to flow past the expandable member in a proximal to distal direction. The method further comprises positioning a fluid port of a catheter between the treated occlusion and the expandable member, delivering irrigation fluid through the fluid port, and using the irrigation fluid to provide fluid flow across the treated occlusion in a distal to proximal direction.

Yet another aspect of the invention comprises a method of treating an occlusion in a blood vessel, comprising positioning an expandable member distal to the occlusion to be treated, using a therapy balloon to perform therapy on the occlusion, using the expandable member to block migration of emboli created as a result of the therapy, while allowing blood to flow from one side to another side of the expandable member in a proximal to distal direction. The method further comprises using the therapy balloon to occlude the blood vessel at a location distal to the treated occlusion, positioning a fluid port of a catheter between the treated occlusion and said location, and providing fluid flow through the fluid port such that said fluid flows across the treated occlusion.

### BRIEF DESCRIPTION OF THE DRAWINGS

FIG. 1 is a perspective view of an integrated inflation/deflation device, shown operably coupled to an illustrative inflation adapter and a balloon catheter deployed in a blood vessel.

FIG. 2A is a side view of a balloon catheter which can be used in accordance with one preferred embodiment of the present invention.

FIG. 2B is a longitudinal cross-sectional view of the distal end of the balloon catheter of FIG. 2A.

FIG. 2C is an enlarged cross-sectional view of the proximal end of the balloon of FIG. 2B.

FIG. 3 shows the inflation adapter of FIG. 1 having a low profile catheter valve and balloon catheter placed therewithin.

FIG. 4A is a partial cross-sectional view of a low profile catheter valve.

FIG. 4B is an enlarged view of the low profile catheter valve of FIG. 4A, showing the valve in an open position (and a closed position shown in phantom).

FIG. 5 is a side view of an illustrative single operator type aspiration catheter according to a preferred embodiment of the present invention.

FIGS. 6A-6D are partial cross-sectional views of a guidewire having an occlusion balloon and an aspiration catheter crossing an occlusion.

FIG. 7 is a perspective view of a therapy catheter delivering a drug and a guidewire having an occlusive device inserted into a blood vessel, with the blood vessel shown partially cut away.

FIGS. 8A and 8B show a catheter having an occlusive device at its distal end, and an aspiration catheter, inserted into a blood vessel to treat an intravascular occlusion using the drug delivery method according to one embodiment of the present invention.

FIG. 9A is a side view of a guidewire having side ports for delivering fluids to an occlusion in a blood vessel, with the vessel shown partially cut away.

FIG. 9B is a side view of a guidewire having an irrigation hole at its distal end for delivering fluids to an occlusion in a blood vessel, with the vessel shown partially cut away..

FIG. 10A is a side view of a temporary occlusion balloon catheter having side ports for delivering fluids to an occlusion in the blood vessel, with the vessel shown partially cut away.

FIG. 10B is a side view of the catheter of FIG. 10A, showing the balloon inflated.

FIGS. 11A-11C are schematic cross-sectional views of alternative embodiments of a hollow catheter having holes, valves, and the like, to permit the escape of irrigation or other fluids.

FIG. 12 is a perspective view of an embodiment in which a distal occlusion device has a plurality of holes therein for passing fluid across an occlusion.

FIG. 13 is a perspective view of an embodiment in which an elongate member (e.g., a guidewire) has a plurality of holes therein for passing fluid across an occlusion.

FIG. 14 is a side-elevational view partially in section showing the catheter apparatus or assembly of the present invention for treating occluded vessels.

FIG. 15 is a cross-sectional view taken along the line 15-15 of FIG. 14.

FIG. 16 is a cross-sectional view taken along the line 16-16 of FIG. 14.

FIG. 17 is a cross-sectional view taken along the line 17-17 of FIG. 14.

FIG. 18 is a schematic illustration of how the catheter apparatus shown in FIG. 19 is deployed in a carotid artery.

FIGS. 19A-19E are illustrations showing the various steps utilized in deployment of the catheter apparatus in performing the method of the present invention in a vessel where a bifurcation is not present.

FIG. 20 is a side-elevational view partially in section of another embodiment of a catheter apparatus or assembly incorporating the present invention for treating occluded vessels using an atherectomy device.

FIG. 21 is a cross-sectional view taken along the line 21-21 of FIG. 20.

FIG. 22 is a cross-sectional view taken along the line 22-22 of FIG. 20.

FIG. 23 is a side-elevational view in section of the distal extremity of another embodiment of a catheter apparatus incorporating the present invention and utilized for delivering an expandable stent to a stenosis.

FIG. 24A is a schematic illustration showing the manner in which the apparatus of the present invention is utilized in connection with vessels of a patient in performing the method of the present invention.

FIG. 24B is an additional partial schematic illustration showing interconnections in the catheter apparatus shown in FIG. 24A.

FIG. 25 is a plan view of another embodiment of a catheter apparatus incorporating the present invention.

FIG. 26 is a cross-sectional view taken along the line 26-26 of FIG. 25.

FIG. 27 is an end elevational view looking down the line 27-27 of FIG. 25.

FIGS. 28A, B, C, and D are illustrations or cartoons showing the method of the present invention being utilized with the apparatus shown in FIG. 24 in a vessel having a bifurcation therein.

FIG. 29 is a side-elevational view of a main catheter incorporating the present invention.

FIGS. 29A and 29B are partial side-elevational views of the distal extremities showing alternative embodiments of the main catheter of the present invention incorporating, respectively, Judkins left shape and Judkins right shape in their distal extremities.

FIG. 30 is a cross-sectional view taken along the line 30-30 of FIG. 29.

FIG. 31 is a cross-sectional view taken along the line 31-31 of FIG. 29.

FIG. 32 is an enlarged partial cross-sectional view of the distal extremity of the catheter shown in FIG. 29.

FIG. 33 is a side-elevational view of the balloon-on-a-wire construction incorporating the present invention.

FIG. 34 is a cross-sectional view taken along the line 34-34 of FIG. 33.

FIG. 35 is an enlarged cross-sectional view of the distal extremity of the construction in FIG. 33.

FIG. 36 is a cross-sectional view similar to FIG. 35 but showing a different embodiment utilizing a twisted dual core.

FIG. 37 is a cross-sectional view similar to FIG. 35 but showing the use of a twisted core.

FIG. 38 is a cross-sectional view of the proximal removable fitting of the construction shown in FIG. 33.

FIG. 39 is a side-elevational view partially in cross section of an irrigation catheter incorporation the present invention.

FIGS. 39A and 39B are side-elevational views of the distal extremities of additional embodiments of irrigation catheters incorporating the present invention.

FIGS. 40-46 are cartoons showing the manner in which the apparatus of the present invention shown in FIGS. 29-39 is used performing a therapeutic procedure in accordance with the present invention.

FIG. 47 is a side-elevational view partially in cross-section of another embodiment of a main catheter incorporating the present invention.

FIG. 48 is a side-elevational view partially in cross-section showing another embodiment of an irrigation catheter incorporating the present invention.

FIGS. 49-53 are cartoons showing the manner in which a therapeutic carotid procedure is performed in accordance with the present invention where there is a bifurcation.

FIG. 54 is a side-elevational view partially in section of another embodiment of a balloon-on-a-wire incorporating the present invention.

FIG. 55 is a cross-sectional view taken along the line 55-55 of FIG. 54.

FIG. 56 is a side-elevational view in section of another embodiment of a catheter apparatus incorporating the present invention for treating occluded vessels.

FIG. 57 is a side-elevational view in section similar to FIG. 56 but showing the apparatus in FIG. 56 with the self- expandable sealing means deployed.

FIG. 58 is a side-elevational view in section of another embodiment of a catheter apparatus incorporating the present invention for treating occluded vessels.

FIG. 59 is a view similar to FIG. 58 but showing the self-expandable sealing means deployed.

FIG. 60A is a schematic illustration of the catheter system of the present invention illustrating the manner in which an emboli containment chamber is formed.

FIG. 60B is a schematic illustration of the catheter system of the present invention utilizing a distal filter.

FIGS. 61A-H illustrate the use of the catheters of the present invention in emboli containment treatment procedures.

FIG. 62 is a perspective drawing of the carotid arteries.

FIG. 63 is a side view of an embodiment of a main catheter.

FIG. 64 is a cross-sectional view of the main catheter taken along line 64-64 of FIG. 63.

FIG. 65 is a cross-sectional view of the main catheter taken along line 65-65 of FIG. 63.

FIG. 66 is a side view of the distal end of an embodiment of an inner catheter.

FIG. 67 is a partial cross-sectional view of the inner catheter taken along line 67-67 of FIG. 66.

FIG. 68 is a side view of an embodiment of an over-the-wire aspiration catheter.

FIG. 69 is a cross-sectional view of the over-the-wire aspiration catheter taken along line 69-69 in FIG. 68.

FIG. 70 is a cross-sectional view of the over-the-wire aspiration catheter taken along line 69-69 in FIG. 68, showing an elongate member (e.g., a guidewire) inserted therethrough.

FIG. 71 is a side view of an embodiment of a single operator aspiration catheter.

FIG. 72 is a cross-sectional view of the single operator aspiration catheter taken along line 72-72 in FIG. 71.

FIG. 73 is a side view of an embodiment of an over-the-wire irrigation or aspiration catheter.

FIG. 74 is a side view of an embodiment of a single operator irrigation catheter.

FIGS. 75 through 77A are cross-sectional views of the single operator irrigation catheter taken along lines 75-75, 76-76 and 77A-77A of FIG. 74.

FIG. 78 is a perspective view of one example of an emboli containment and removal method within a carotid artery.

FIG. 79A is a perspective view of another example of an emboli containment and removal method.

FIG. 79B is a perspective view of an emboli containment and removal method with a filter in the internal carotid artery.

FIG. 80 is a perspective view of yet another example of an emboli containment and removal method which employs a single occlusive device.

FIG. 81 is a perspective view of the emboli containment and removal method illustrated in FIG. 80, showing the use of an intermediate catheter.

FIG. 82 is a perspective view of still another example of an emboli containment and removal method which employs two occlusive devices.

FIG. 83 is a perspective view showing a preferred location for the intermediate catheter when the intermediate catheter is used to flush away emboli from the treated occlusion.

FIG. 84 is a perspective view of an embodiment in which the intermediate catheter is used for aspiration of emboli.

FIG. 85 is a perspective view of an embodiment in which the intermediate catheter is used for irrigation of emboli.

FIG. 85A is a perspective view of an embodiment in which a therapy catheter is used for irrigation of emboli.

FIG. 86 is a perspective view of an embodiment in which the main catheter is used for aspiration of emboli.

FIG. 87 is a perspective view of an embodiment in which the main catheter is used for irrigation of emboli.

FIG. 88 is a perspective view of an embodiment in which a distal occlusion device has a plurality of holes therein for passing irrigation fluid across the treated occlusion.

FIG. 89 is a perspective view of an embodiment in which an elongate member (e.g., a guidewire) has a plurality of holes therein for passing irrigation fluid across the treated occlusion.

FIGS. 90A and 90B are perspective views of an embodiment in which a perfusion-filter located distal to the lesion to be treated permits the perfusion of blood while entraining emboli produced as a result of therapy.

FIG. 91A is a perspective view of an alternative embodiment in which emboli are captured in a perfusion-filter.

FIG. 91B is a cross sectional view of the main catheter of the embodiment of FIG. 91A, illustrating lumens used for inflation of the occlusive device, for delivering irrigation fluid, and for passing an elongate member (e.g., a guidewire).

FIG. 92 is a perspective view of an embodiment in which passive perfusion is performed with a hypotube having holes therein.

FIGS. 93A and 93B are perspective views of a perfusion-filter embodiment in which the occlusion of the vessel is performed with the therapy catheter.

FIG. 94 shows a preferred embodiment of a syringe assembly having features in accordance with the present invention and operably coupled to an illustrative inflation adapter at a proximal portion of a balloon guidewire catheter.

FIGS. 95A and 95B show open and closed positions, respectively, of the sealing member, which is used with the balloon guidewire catheter of FIGS. 94 and 96.

FIG. 96 shows a perspective view of the balloon guidewire catheter of FIG. 94 placed within an open inflation adapter.

### DETAILED DESCRIPTION OF THE PREFERRED EMBODIMENTS

Certain preferred embodiments of the present invention provide methods for localized drug delivery in high concentration to the site of an intravascular occlusion by using an aspiration catheter for both aspiration and drug delivery. This method is used either alone, or in combination with a therapy catheter as discussed below. The drug delivery method may be used in conjunction with any method for preventing distal embolization during removal of plaque, thrombi or other occlusions from a blood vessel. A preferred embodiment of the present invention is adapted for use in the treatment of a stenosis or an occlusion in a blood vessel in which the stenosis or occlusion has a length and a width or thickness which at least partially occludes the vessel's lumen. Thus, the method is effective in treating both partial and complete occlusions of blood vessels.

It is to be understood that “occlusion” as used herein with reference to a blood vessel is a broad term and is used in its ordinary sense and includes both complete and partial occlusions, stenoses, emboli, thrombi, plaque and any other substance which at least partially occludes the lumen of the blood vessel. The term “occlusive device” as used herein is a broad term and is used in its ordinary sense and includes balloons, filters and other devices which are used to partially or completely occlude the blood vessel prior to performing therapy on the occlusion. It will be appreciated that even when a filter is used, the filter may be partially or completely occlusive.

The term “drugs” as used herein is a broad term and is used in its ordinary sense and includes genes and cells. The methods of the present invention are particularly suited for use in removal of occlusions from saphenous vein grafts, coronary and carotid arteries, and vessels having similar pressures and flow.

### I. Overview of Occlusion System

A. Balloon System

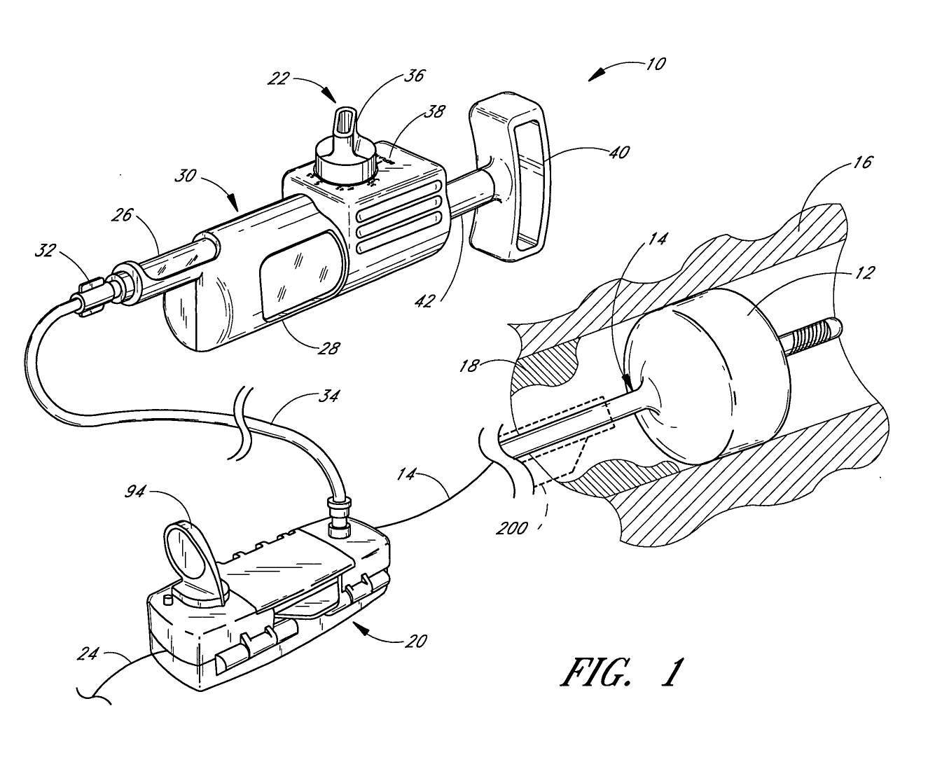

FIG. 1 illustrates generally the components of one exemplifying occlusion balloon guidewire system 10. As described in further detail below, an occlusion balloon 12 used in this system is delivered on a guidewire 14 to a location in a blood vessel 16 distal an occlusion 18. Through the use of an adapter 20 and an inflation/deflation device or syringe assembly 22, the balloon is inflated through a lumen in the guidewire 14 to occlude the vessel distal to the occlusion. Through the use of a valve 24 described below, the adapter 20 can be removed from the proximal end of the guidewire 14 while the balloon 12 remains inflated. With the proximal end of the guidewire free of obstructions, various therapy and other catheters can be delivered and exchanged over the guidewire 14 to perform treatment on the occlusion 18. Because the balloon 12 on the guidewire 14 remains inflated distal to the occlusion 18, any particles broken off by treating the occlusion 18 are isolated proximal to the balloon. These particles can be removed using an aspiration catheter 200 (shown in phantom in FIG. 1) delivered over the guidewire. After the particles are removed, the adapter 20 and inflation/deflation device 22 can be reattached to the proximal end of the guidewire to deflate the balloon.

B. Syringe Assembly

Preferred embodiments of the present invention may comprise or be used in conjunction with a syringe assembly as described in U.S. Pat. No. 6,234,996, the entirety of which is incorporated herein by reference in its entirety. One preferred syringe assembly is available from Medtronic PercuSurge, Inc. of Sunnyvale, Calif. under the name EZ FLATOR™.

One preferred embodiment of a syringe assembly 22 for inflation and deflation of an occlusion balloon is shown in FIG. 1. The syringe assembly 22 comprises a low-volume inflation syringe 26 and a high capacity or reservoir syringe 28 encased together in a housing 30. The syringe assembly 22 is preferably attached via a connector 32 and a short tube 34 to an adapter 20 within which a low profile catheter valve 24 and a balloon catheter 14 are engaged during use. The balloon catheter is shown in an inflated state within a blood vessel in FIG. 1 An inflation/deflation knob 36 is disposed on the outside of the housing 30. Indicia 38 are preferably located on the housing 30 adjacent the knob 36 so that a clinician using the device can monitor the precise volume of liquid delivered by the inflation syringe 22. As depicted, the indicia 38 preferably comprise numbers corresponding to the size and shape of the balloon used. When the knob 38 is rotated from the “DEFLATE” or “0:” position to the number corresponding to the balloon in use, the syringe assembly 22 delivers the fluid volume associated with that balloon size. Alternatively, the indicia 38 could indicate the standard or metric volume of fluid delivered at each position. A handle 40 is formed at a proximal end of the plunger 42. Preferably, the handle 40 is large, as illustrated in FIG. 1, and is easily held in a clinician's hand.

C. Occlusion Balloon Guidewire

The occlusion balloon guidewire system generally illustrated in FIG. 1 performs the function of occluding a vessel and allowing for the slidable insertion or advancement of various other catheters and devices. The term “catheter” as used herein is therefore intended to include both guidewires and catheters with these desired characteristics.

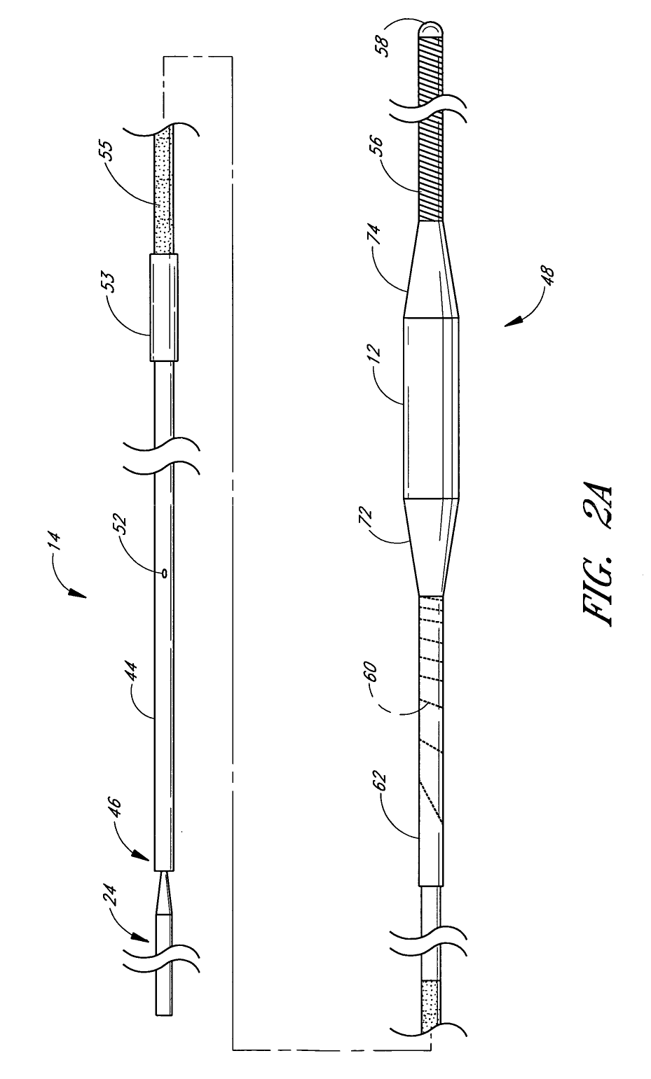

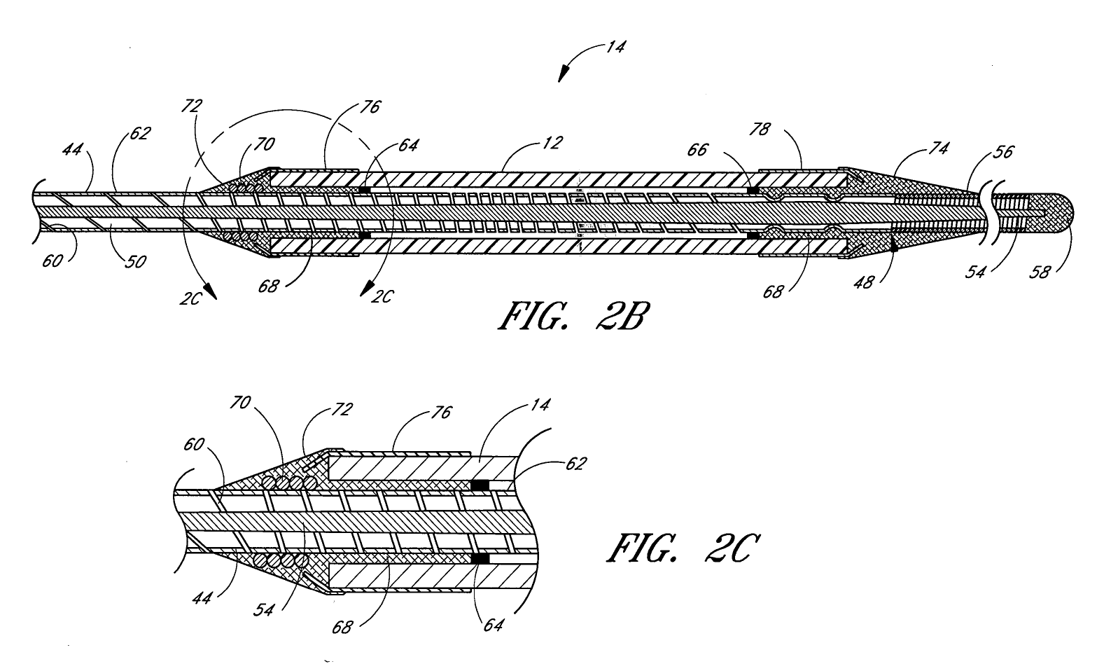

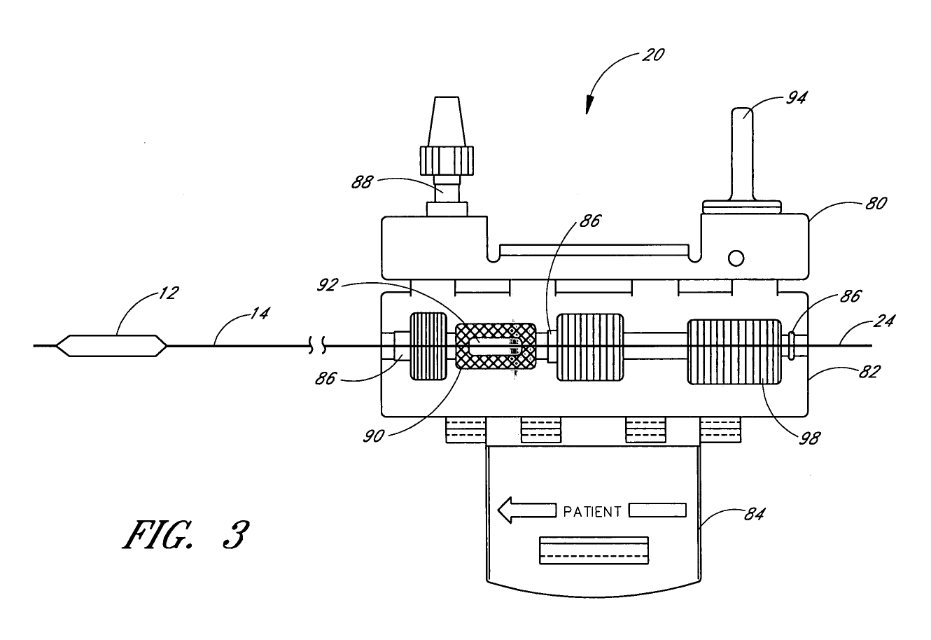

As shown in FIG. 2A, a balloon guidewire catheter 14 generally comprises an elongate flexible tubular body 44 extending between a proximal control end 46, corresponding to a proximal section of the tubular body 44, and a distal functional end 50 (not shown), corresponding to a distal section of tubular body 44. Tubular body 44 has a central lumen 48, which extends between the proximal and distal ends. An inflation port 52, shown also in FIGS. 4A and 4B described below, is provided on tubular body 44 near the proximal end 46. Inflation port 52 is in fluid communication with lumen 50 such that fluid passing through inflation port 52 into or out of the lumen 50 may be used to inflate or deflate an inflatable balloon 12 in communication with lumen 50.

A valve 24, as described below, is inserted into the proximal end 46 of the tubular body 44 to control inflation of a balloon 12 mounted on the distal end of the tubular body through inflation notch 52. The inflation notch 52 is preferably formed by electric discharge machining (EDM). A proximal marker 53, which is preferably made of gold, is placed over the tubular body 44 distal to the inflation notch 52. Distal to the marker 53, a nonuniform coating 55 of polymer material, more preferably polytetrafluoroethylene (TFE), is applied to the tubular body 44, terminating proximal to a shrink tubing 62. The shrink tubing 62 extends up to and within the balloon 12, as described below. Adhesive tapers 72 and 74 extend from the proximal and distal ends of the balloon, respectively. The proximal taper 72 preferably extends from the proximal end of the balloon to the shrink tubing 62 on the tubular body 44, while the distal taper 74 extends to coils 56 extending from the distal end 48 of the tubular body 44. The coils 52 terminate in a distal ball 58.

The length of the tubular body 44 may be varied considerably depending on the desired application. For example, when catheter 14 serves as a guidewire for other catheters in a conventional percutaneous transluminal coronary angioplasty procedure involving femoral artery access, tubular body 44 is comprised of a hollow hypotube having a length in the range from about 160 to about 320 centimeters, with a length of about 180 centimeters being optimal for a single operator device, or 300 centimeters for over the wire applications. Alternatively, for a different treatment procedure not requiring as long a length of tubular body 44, shorter lengths of tubular body 44 may be provided.

Tubular body 44 generally has a circular cross-sectional configuration with an outer diameter within the range from about 0.008 inches to 0.14 inches. In applications where catheter 14 is to be used as a guidewire for other catheters, the outer diameter of tubular body 44 ranges from 0.010 inches to 0.038 inches and preferably is about 0.014 to 0.020 inches in outer diameter or smaller. Noncircular cross-sectional configurations of lumen 50 can also be adapted for use with the catheter 14. For example, triangular, rectangular, oval and other noncircular cross-sectional configurations are also easily incorporated for use with the preferred embodiments, as will be appreciated by those of skill in the art. The tubular body 44 may also have variable cross-sections.

The tubular body 44 has sufficient structural integrity or “pushability” to permit catheter 14 to be advanced through the vasculature of a patient to distal arterial locations without buckling or undesirable kinking of tubular body 44. It is also desirable for the tubular body 44 to have the ability to transmit torque such as in those embodiments where it may be desirable to rotate tubular body after insertion into a patient. A variety of biocompatible materials known by those of skill in the art to possess these properties and to be suitable for catheter manufacture may be used to produce tubular body 44. For example, tubular body 44 may be made of a stainless steel material such as ELGILOY™. or may be made of polymeric material such as PEEK, nylon, polyimide, polyamide, polyethylene or combinations thereof. In one preferred embodiment, the desired properties of structural integrity and torque transmission are achieved by forming the tubular body 44 out of an alloy of titanium and nickel, commonly referred to as nitinol. In a more preferred embodiment, the nitinol alloy used to form the tubular body 80 is comprised of about 50.8% nickel and the balance titanium, which is sold under the trade mark TINEL™ by Memry Corporation. It has been found that a catheter tubular body having this composition of nickel and titanium exhibits an improved combination of flexibility and kink-resistance in comparison to other materials.

Other details regarding construction of balloon guidewire catheters may be found in assignee's U.S. Pat. Nos. 6,068,623, 6,228,072, and copending applications entitled FLEXIBLE CATHETER, application Ser. No. 09/253,591, filed Feb. 22, 1999, now U.S. Pat. No. 6,500,147, and FLEXIBLE CATHETER WITH BALLOON SEAL BANDS, application Ser. No. 09/653,217, filed Aug. 31, 2000, now abandoned, all of which are hereby incorporated by reference in their entirety. One preferred guidewire system is available from Medtronic PercuSurge, Inc. of Sunnyvale, Calif., under the name GUARDWIRE PLUS™.

As illustrated in FIG. 2A, an occlusive device such as an inflatable balloon 12 is mounted on the distal end 48 of tubular body 44. In one preferred embodiment, the balloon 12 is a compliant balloon formed of a material comprising a block polymer of styrene-ethylene-butylene-styrene (SEBS), as disclosed in assignee's copending application entitled BALLOON CATHETER AND METHOD OF MANUFACTURE, application Ser. No. 09/026,225, filed on Feb. 19, 1998, now U.S. Pat. No. 6,554,795, and in U.S. Pat. No. 5,868,705, the entirety of both of which are hereby incorporated by reference. The balloon 12 may be secured to the tubular body 44 by any means known to those skilled in the art, such as adhesives or heat bonding. For example, for attachment of a SEBS balloon to a nitinol tube, a primer such as 7701 LOCTITE™ by Loctite Corporation is preferably used along with cyanoacrylate adhesive such as LOCTITE-4011.

The balloon 12 described in the preferred embodiments preferably has a length of about 5 to 9 mm and more preferably about 6 to 8 mm. Other occlusive devices such as filters are suitable for the catheter 44, such as those disclosed in assignee's copending applications entitled OCCLUSION OF A VESSEL, Ser. No. 09/026,106, filed Feb. 19, 1998, now U.S. Pat. No. 6,312,407, OCCLUSION OF A VESSEL, Ser. No. 09/374,741, filed Aug. 13, 1999, now abandoned, OCCLUSION OF A VESSEL AND ADAPTER THEREFOR, Ser. No. 09/509,911, filed Feb. 17, 2000, now abandoned, MEMBRANES FOR OCCLUSION DEVICE AND METHODS AND APPARATUS FOR REDUCING CLOGGING, Ser. No. 09/505,554, filed Feb. 17, 2000, now abandoned, and STRUT DESIGN FOR AN OCCLUSION DEVICE, Ser. No. 09/505,546, filed Feb. 17, 2000, now abandoned, the entirety of each of which is hereby incorporated by reference.

With reference to FIG. 2B, a core wire 54 is provided inside the lumen 50 and is crimped to the tubular body 44. Coils 56 extend from the distal end of the tubular body 44, surround the core wire 54, and terminate in a distal ball 58. In one embodiment, the core wire may have one or more tapers, and can extend proximally into tubular body 44. Other details regarding the core wire are discussed in assignee's copending application entitled CATHETER CORE WIRE, Ser. No. 09/253,971, filed Feb. 22, 1999, now U.S. Pat. No. 6,355,016, the entirety of which is hereby incorporated by reference.

In one embodiment, shown in FIG. 2B, the tubular body 44 preferably has cuts 60 to create a coiled configuration. A sleeve 62 is preferably provided over the tubular body 44. Adhesive stops 64 and 66 are provided about 1 to 2 mm from the ends of the balloon, to control the wicking length of the adhesive 68 into the balloon working area. Balloon inflation is provided through the cuts 60 in the tubular body 44. A marker 70 is mounted to the tubular body 66 proximal of the balloon 12. Adhesive tapers 72 and 74 are provided adjacent the balloon 12 to provide a transition region between the tubular body 44 and balloon 12 at the balloon's proximal end and between the balloon 12 and the core wire 54 at the balloon's distal end. Seal bands 76 and 78 are applied to the proximal and distal ends of the balloon to improve bond integrity. Other details regarding this balloon catheter may be found in assignee's above-referenced copending applications entitled FLEXIBLE CATHETER and FLEXIBLE CATHETER WITH BALLOON SEAL BANDS.

D. Inflation Adapter and Low Profile Catheter Valve

Referring next to FIG. 3, the inflation adapter 20 comprises a housing having two halves 80, 82 preferably formed of metal, medical grade polycarbonate, or the like. The halves 80, 82 are attached by hinges to be separated or joined in a clam shell manner. A locking clip 84 secures the halves while the adapter 20 is in use. Clips 82 within the housing accept and securely hold the catheter 14 in a correct position. The male luer member 88 or another suitable connector, extends from a top of the housing to provide an inflation passageway. Seals 90 are provided within the housing and around an internal segment 92 of the inflation pathway to conduct the pressurized fluid provided by the syringe assembly 22. An actuator 94, shown in FIG. 1 at the top of the adapter housing 96, controls a cam which operates sliding panels 98 (FIG. 3) contained in the housing.

As shown in FIG. 1, a low profile catheter valve 24 is attached to an open proximal end of the catheter 14. Inflation fluid is injected through the adapter 20 and valve 24 into a lumen of the hollow catheter 14, and into the balloon 12. The inflation adapter 20 is used to open and close the valve 24 to regulate the inflation of the balloon 12 mounted on the distal end of the catheter 14.

It will be emphasized that other types of adapters and/or valves can be employed with the inflation syringe and/or syringe assembly described herein, in order to achieve rapid and accurate inflation/deflation of medical balloons or other non-balloon medical devices. Therefore, although the preferred embodiments are illustrated in connection with a low volume occlusion balloon 12, other types of balloons and non-balloon devices can benefit from the advantages of the invention described herein.

As shown in FIGS. 4A and 4B, the low profile catheter valve 24 comprises a movable sealer portion 100 attached at a distal end of a wire segment 102 and positioned within the inflation lumen 50 of the guidewire catheter 14. The wire 102 may be secured to a spring just within a proximal opening of the catheter 14. It will be noted that various spring or biasing arrangements may be utilized, including a zig-zag wire 104 which is formed on or replaces the wire segment 102 and which provides biasing force to the sealer portion 100 due to frictional engagement with the walls of the lumen 50. The sealer portion 100 forms a fluid tight seal with the inflation lumen 50 by firmly contacting the entire circumference of a section of the inflation lumen 50. The sealer portion 100 may be positioned proximally of the side-access inflation port 90 on the catheter as shown in FIG. 4B, to establish an unrestricted fluid pathway between the inflation port 52 and the inflatable balloon on the distal end. As desired, the clinician may move the sealer portion 100 to a position at or distal of the inflation port 52, as shown in phantom in FIG. 4B, thereby preventing any fluid from being introduced into or withdrawn from the lumen 50 via the inflation port 52. The valve 24 is considered “low profile” because it is no larger in cross-sectional diameter than the catheter 14 itself.

Preferably, the catheter 14 is positioned within the housing of the adapter 20 with the valve closed, such that the side inflation port 52 is located in the sealed inflation area 92 of the housing. The catheter 14 is then positioned in the second half 82 of the adapter 20. A distal portion of the catheter 14 extends out of the housing and into the patient, and a proximal portion of the catheter including the catheter valve 24 extends out of the other side of the adapter 20. The adapter is closed, the locking clip 84 is secured, and a syringe assembly is attached. The actuator 94 is moved from a first position to a second position, such that the sliding panels 98 within the housing cause the valve 24 to be in an open position to allow fluid flow through the inflation port 52. A syringe assembly 22 is then used to inflate the balloon 12. Closing the valve 24 is accomplished by moving the actuator 96 from the second position back to the first position, such that the balloon inflation is maintained. Once the valve is closed the adapter may be removed and treatment and other catheters may be delivered over the guidewire.

Other inflation adapter/inflation syringe assemblies may also be used. Also, the adapter 20 can have additional features, such as a safety lock provided on the actuator knob 94 to prevent accidental opening when the adapter is being used and the catheter valve is open. In addition, the adapter can be provided with an overdrive system to overdrive a sealing member into a catheter. Details of these features and other inflation assemblies may be found in assignee's U.S. Pat. No. 6,050,972 and copending applications, SYRINGE AND METHOD FOR INFLATING LOW PROFILE CATHETER BALLOONS, application Ser. No. 09/025,991, filed Feb. 19, 1998, now abandoned, and LOW VOLUME SYRINGE AND METHOD FOR INFLATING SURGICAL BALLOONS, application Ser. No. 09/195,796, filed Nov. 19, 1998, now abandoned, all of which are incorporated by reference in their entirety.

E. Aspiration Catheter

The occlusion system described above advantageously enables an exchange of catheters over a guidewire while an occlusive device isolates particles within the blood vessel. For example, a therapy catheter can be delivered over the guidewire to perform treatment, and then be exchanged with an aspiration catheter to remove particles from the vessel. Further details of this exchange are described in assignee's copending application entitled EXCHANGE METHOD FOR EMBOLI CONTAINMENT, Ser. No. 09/049,712, filed Mar. 27, 1998, now U.S. Pat. No. 6,544,276, the entirety of which is hereby incorporated by reference.

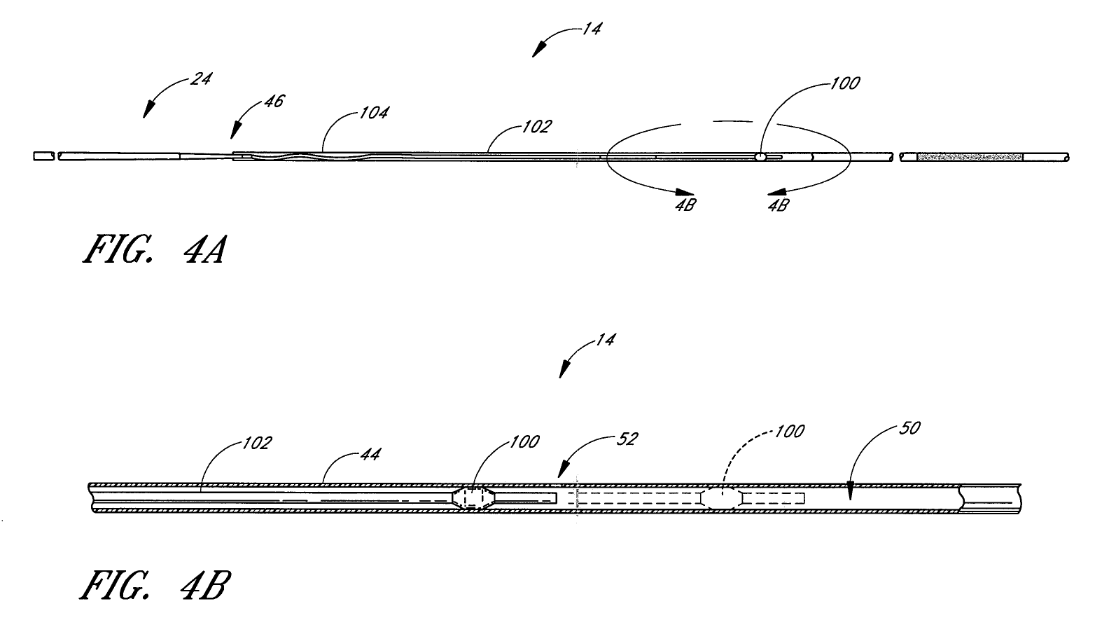

An aspiration catheter according to one preferred embodiment of the present invention is shown in FIG. 5. The catheter 200 includes an adapter 202 and an aspiration port 204 at its proximal end to which a source of negative pressure is attached. The aspiration catheter further comprises an elongate tubular body 206 which extends distally from the adapter 202 and through a plurality of support sheaths 210 and 212. Beyond the support sheath 212 the elongate tubular body 206 extends to a transition point 214 where the outer diameter of the tubular body 206 tapers down in size. This tapered or necked-down portion of the tubular body 206 is preferably inserted into a dual lumen tubing 216 through the proximal end 218 of the dual lumen tubing. The tubular body 206 is preferably inserted into one of the lumens of the dual lumen tubing 216 such that its distal end 220 is a sufficient distance distal from the proximal end 218 of the dual lumen tubing to provide a secure connection therebetween.

The dual lumen tubing 216 preferably defines two lumens, one for aspiration and the other for a guidewire to pass therethrough. More particularly, the lumen that the elongate body 206 is inserted into acts as the aspiration lumen, being in fluid communication with the lumen of the elongate tubular body 206. The aspiration lumen preferably ends in a distal aspiration mouth 222, which preferably defines an oblique opening. Aspiration therefore occurs through both the lumen of the elongate tubular body 206 and the aspiration lumen of the dual lumen tubing.

The guidewire lumen is provided adjacent the aspiration lumen in the dual lumen tubing and has a proximal end 224 preferably distal to the proximal end 218 of the aspiration lumen of the dual lumen tubing, and a distal end 226 preferably distal to the aspiration mouth 222. A marker 228 is placed within the guidewire lumen at the distal end of the aspiration mouth. Additional markers 230, 232 may also be placed over the elongate body 206 and/or support sheaths. Further details regarding these and other aspiration catheters are provided below and in Applicant's copending applications entitled ASPIRATION CATHETER, Ser. No. 09/454,522, filed Dec. 7, 1999, now U.S. Pat. Nos. 6,849,068, and 6,152,909, the entirety of both of which are hereby incorporated by reference.

### II. Drug Delivery and Other Treatment Methods

In a preferred embodiment of the invention, an occlusion balloon guidewire 14 such as described above is delivered to the site of an occlusion in a blood vessel. In one embodiment (not shown), a guide catheter is first introduced into the patient's vasculature through an incision made in the femoral artery in the groin and is used to guide the insertion of the guidewire and/or other catheters and devices to the desired site. The guidewire is then advanced until its distal end reaches a site proximal to the occlusion. Fluoroscopy is typically used to guide the guidewire and other devices to the desired location within the patient. The devices are frequently marked with radiopaque markings to facilitate visualization of the insertion and positioning of the devices within the patient's vasculature. It should be noted that at this point, blood is flowing through the vessel in a proximal to distal direction. The guide catheter may then be removed, or alternatively, may be used as the aspiration catheter itself, as described below.

A. Aspirating While Crossing the Occlusion

In one embodiment, aspiration is performed while advancing a guidewire across the site of the occlusion in a proximal to distal direction to prevent distal embolization. An aspiration catheter, such as described below, is delivered over the guidewire to a site just proximal to the site of the occlusion, and, while aspirating, the occlusion in the vessel is crossed with both the guidewire and the aspiration catheter in a proximal to distal direction. Further details of this method are described in assignee's copending application entitled METHODS FOR REDUCING DISTAL EMBOLIZATION, Ser. No. 09/438,030, filed Nov. 10, 1999, now U.S. Pat. No. 6,652,480, and in U.S. Pat. No. 5,833,650, the entirety of both of which are hereby incorporated by reference. The term “aspiration catheter” is intended to include any elongated body having a lumen which can be used to withdraw particles, fluid or other materials from a blood vessel. Any such device can be attached to a suction apparatus for removal of intravascular particles.

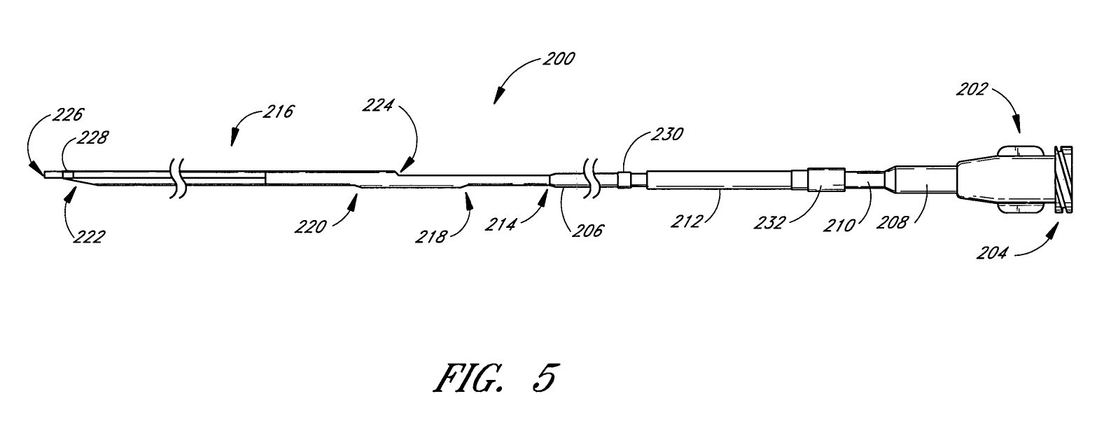

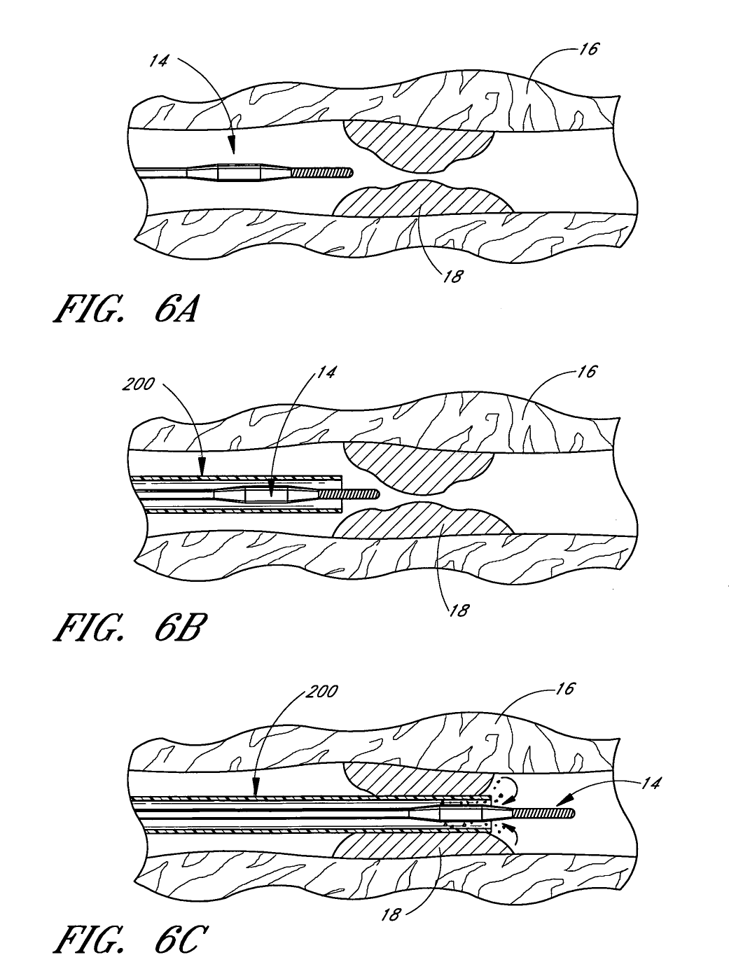

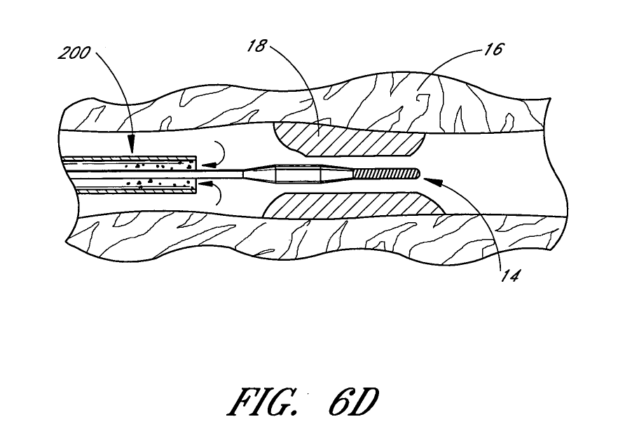

FIGS. 6A-6D illustrate one embodiment in which an occlusion 18 in a vessel 16 is crossed with a guidewire having an occlusive device and an illustrative aspiration catheter 200. It will be appreciated, however, that the occlusion 18 may first be crossed with an ordinary guidewire as described in the above-referenced application METHODS FOR REDUCING DISTAL EMBOLIZATION. A guidewire 14 with an occlusive device such as a balloon at its distal end is inserted into the vessel 16 to a location just proximal to the occlusion 18 (FIG. 6A). An aspiration catheter 200 is delivered over the guidewire 14 so that the distal ends of the guidewire 14 and aspiration catheter 200 are both just proximal to the occlusion 18 (FIG. 6B). Alternatively, the aspiration catheter can be delivered first. Aspiration is performed while crossing or advancing past the occlusion 18 with the distal ends of the guidewire 14 and aspiration catheter 200, in a proximal to distal direction (FIG. 6C). Then the distal end of the aspiration catheter 200 is moved back in a distal to proximal direction while aspirating (FIG. 6D). Blood flow into the aspiration catheter 200 is indicated by the arrows. The proximal to distal, then distal to proximal aspiration may be repeated one or more times if desired.

In one embodiment, the distal tip of the aspiration catheter is no more than about 2 cm, in another embodiment no more than about 0.5-1 cm, behind or proximal to the distal tip of the guidewire during crossing. In yet another embodiment, the distal end of the aspiration catheter is then moved in a distal to proximal direction across the occlusion, while continuously aspirating. This process ensures the removal of any particles which may be created during the delivery of the guidewire to a position distal to at least a portion of the occlusion. Aspiration from proximal to distal, and distal to proximal, can be repeated as many times as necessary to completely aspirate all particles. These procedures are all preferably performed prior to occlusion of the vessel at a site distal to the occlusion with the occlusion device, and prior to treatment of the occlusion. It should be noted that, as used herein, “proximal” refers to the portion of the apparatus closest to the end which remains outside the patient's body, and “distal” refers to the portion closest to the end inserted into the patient's body.

As the guidewire and aspiration catheter cross the occlusion, blood and/or other fluid enters the vessel and keeps any particles dislodged during the procedure from flowing in a distal to proximal direction. In addition, the blood pressure and flow provides the irrigation necessary for aspiration. The blood pressure in the vessel is preferably at least about 0.2 psi, and the vessel is capable of providing a flow rate of at least about 5 cc per minute when not occluded.

B. Drug Delivery

In a drug or fluid delivery embodiment of the present invention, after the distal end of the guidewire having an occlusive device such as a balloon or filter is delivered past the site of the occlusion and the optional aspiration step is complete, the occlusive device is actuated to at least partially, an in one embodiment totally, occlude the vessel at a site distal to the site of the occlusion. In another embodiment, prior to actuation of the occlusive device, a first therapy or other catheter is delivered over the guidewire. Once the blood vessel is occluded, therapy can be performed by delivering a drug or fluid through a catheter advanced over the guidewire to the site of the occlusion as described herein to partially or totally dissolve the occlusion. After therapy has been performed, aspiration of any particles broken off from the occlusion may also be performed while the occlusive device is actuated. It will be appreciated that it may take time for the drug to dissolve or act on the occlusion, and therefore a clinician may wait a desired period before aspirating.

Various thrombolytic or other types of drugs can be delivered locally in high concentrations to the site of the occlusion via a therapy catheter. It is also possible to deliver various chemical substances or enzymes via a therapy catheter to the site of the stenosis to dissolve the obstruction. The therapy catheter can be any of a number of devices that may or may not ride over the guidewire, including a balloon catheter used to perform angioplasty, a catheter which delivers a stent, an atherectomy device, a laser or ultrasound device used to ablate the occlusion and similar devices. Drug delivery using a therapy catheter is shown in FIG. 7.

Referring to FIG. 7, once the vessel 16 is occluded with the occlusion guidewire 14, a therapy catheter 300 is used to treat the occlusion 18. The therapy catheter can be any of a number of devices, including a balloon catheter used to perform angioplasty, a catheter which delivers a stent, a catheter for delivering enzymes, chemicals, or drugs to dissolve and treat the occlusion (as illustrated in FIG. 7), an atherectomy device, or a laser or ultrasound device used to ablate the occlusion. Alternatively, the therapy catheter can be eliminated and use of the guide catheter or a separate aspiration catheter alone can be used to aspirate the occlusion. This method is especially useful to remove emboli from the coronary arteries or saphenous vein graft following acute myocardial infarction, because the aspiration catheter can be made small enough to enter the coronary arteries.

Thus, as illustrated in FIGS. 8A and 8B, in one embodiment, both therapy and aspiration are preferably performed using the same catheter, which is preferably an aspiration catheter 200. Although aspiration catheter 200 as shown in FIGS. 8A and 8B has only one lumen, it will be appreciated that other types of aspiration catheters may be used. For example, an aspiration catheter such as described in FIG. 5 can be employed. Other aspiration catheters are described in U.S. Pat. No. 6,152,909.

In the embodiment where an aspiration catheter 200 aspirates while the guidewire 14 crosses the occlusion 18 as described above, when the occlusive device is actuated the aspiration catheter is already delivered to the site of the occlusion over the guidewire. It will also be appreciated, however, that the guidewire 14 may cross the occlusion 18 without aspirating simultaneously. In this embodiment, the aspiration catheter 200 may be delivered after the guidewire crosses the occlusion. The aspiration catheter is then preferably delivered until it is proximal to the occlusion 18 before the occlusive device such as a balloon is actuated. By actuating the occlusive device before the aspiration catheter crosses the occlusion, the risk of particles migrating downstream during crossing of the occlusion by the aspiration catheter is eliminated. Alternatively, if there is minimal risk that the crossing of the aspiration catheter will break off particles, the occlusive device can be actuated after the aspiration catheter crosses the occlusion 18. As shown in FIG. 8A, once delivered, aspiration catheter 200 is preferably proximal to the balloon 12 and distal to the occlusion 18.

One embodiment relates to localized delivery of high concentrations of a thrombolytic, anticoagulant or restenosis-inhibiting drug through the lumen of the aspiration catheter, to promote dissolution of the occlusion and restoration of blood flow through the blood vessel. The fluid containing the drug which is delivered from the aspiration catheter travels in a proximal to distal direction out of the lumen of the aspiration catheter, as indicated by arrows 234 in FIG. 8A, and then in a distal to proximal direction after contacting the occlusive device, and displaces blood proximally. Additionally, blood flow in the vessel in a proximal to distal direction localizes the drug containing fluid to the area of the occlusion.

Thrombolytic agents contemplated for use in the preferred embodiments of the present invention include, but are not limited to, tissue plasminogen activator (t-PA), streptokinase. Anticoagulants include heparin, hirudin and coumadin. In addition, solutions such as phosphate-buffered saline (PBS), lactated Ringer's solution, or any other pharmaceutically acceptable solution may be used to deliver a radioisotope to the site of an occlusion which has been treated with a therapy catheter to inhibit restenosis of the occlusion. These radioisotopes, including beta-emitters (e.g., ³²p) and gamma-emitters (e.g., ¹³¹I), and any other medically acceptable radioisotopes well known in the art, permanently damage the treated occlusion and prevent tissue regrowth.

Other therapeutic or other agents that may be used include, but are not limited to, thrombin inhibitors, antithrombogenic agents, fibrinolytic agents, cytostatic agents, vasospasm inhibitors, calcium channel blockers, vasodilators, antihypertensive agents, antimicrobial agents, antibiotics, inhibitors of surface glycoprotein receptors, antiplatelet agents, antimitotics, microtubule inhibitors, anti secretory agents, actin inhibitors, remodeling inhibitors, antisense nucleotides, antimetabolites, antiproliferatives, anticancer chemotherapeutic agents, anti-inflammatory steroid or non-steroidal anti-inflammatory agents, immunosuppressive agents, growth hormone antagonists, growth factors, dopamine agonists, radiotherapeutic agents, peptides, proteins, enzymes, extracellular matrix components, inhibitors, free radical scavengers, chelators, antioxidants, anti polymerases, antiviral agents, photodynamic therapy agents, and gene therapy agents.

In one embodiment, the drug is delivered through the lumen of the aspiration catheter at a flow rate of between about 0.1 cc/sec and 10 cc/sec, in another embodiment, about 0.5 to 2 cc/sec, and in yet another embodiment, about 0.5 cc/sec to 1 cc/sec. In another embodiment, the tip of the aspiration catheter is placed about 0.5 mm to 10 mm, more preferably about 1 mm to 5 mm, from the surface of the occlusive device. Localization of the tip of the aspiration catheter close to the occlusive device 12 creates a more isolated area for drug treatment of the occlusion. In one embodiment, when the tip of the aspiration catheter is close to the surface of the occlusive device, the fluid containing the drug replaces the column of blood distal to the catheter tip, resulting in proximal to distal movement of the fluid containing the drug which replaces the column of blood distal to the catheter tip. In contrast, if the tip of the catheter is placed too far proximal to the occlusive device, the fluid containing the drug cannot move forward out of the catheter due to the force exerted by the column of blood distal to the catheter tip.

In another embodiment, when the drug delivered through the lumen of the aspiration catheter is released at a rapid rate, the drug moves in a proximal to distal direction toward the occlusive device. Once the drug reaches the occlusive device, at least a portion of the drug bounces against the occlusive device and moves in a distal to proximal direction. This localizes the drug at a location proximal to the occlusive device.

After drugs are delivered through the aspiration catheter 200, emboli or other particles 236 may be formed in the vessel as shown in FIG. 8B. Aspiration can then occur through the same lumen that delivered drugs to the occlusion 18, as indicated by arrows 238. The aspiration catheter may preferably be moved proximally and distally in order to optimize aspiration. The use of the same aspiration catheter lumen advantageously reduces the time that the occlusive device remains expanded, thereby minimizing risk to the patient. Once aspiration is complete, the occlusive device can be deactivated to restore blood flow to the vessel. Further details regarding aspirating particles are described in U.S. Pat. No. 6,135,991, the entirety of which is incorporated by reference. Following aspiration, additional therapy can be performed using a therapy catheter if desired. When separate therapy and aspiration catheters are used, once the desired therapy is performed, the therapy catheter is withdrawn from the patient's body and the aspiration catheter can once again be delivered over the guidewire.

The aspiration catheter, as shown in FIGS. 8A and 8B, rides over the guidewire with the guidewire inserted through the aspiration lumen of the catheter. Alternatively, a single operator type aspiration catheter can be used, in which only a portion of the aspiration catheter rides over the guidewire, which is inserted into a separate guidewire lumen. Single operator catheters suitable for use with these embodiments are described below.

C. Irrigation Catheters

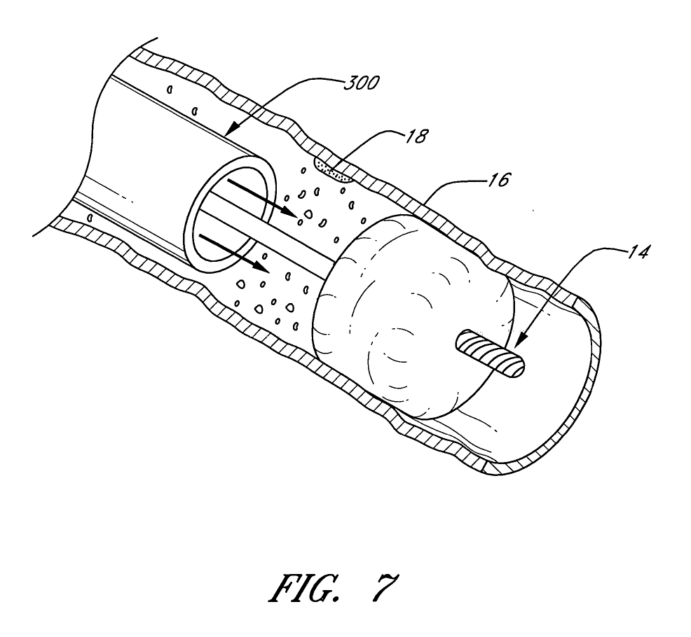

It will be appreciated that when the occlusion in the vessel is too large, it is often desirable to create some space to move past the occlusion prior to delivering the guidewire 14 having the occlusive device. To do this, a guidewire 14 without a balloon or other occlusive device may be used which contains side ports 240 near the distal end and/or an irrigation hole 242 at its distal end, as shown in FIGS. 9A and 9B, respectively. Fluids such as described above are ejected through these holes to break apart the occlusion as the guidewire crosses the occlusion. Further details describing guidewires for fluid delivery are contained in assignee's U.S. Pat. No. 6,068,623. After the guidewire has cleared space through the occlusion, in one embodiment a therapy catheter or aspiration catheter as described above can simply be delivered over this catheter to perform treatment on the occlusion. It will also be appreciated that an aspiration catheter may simultaneously be used to aspirate particles broken off from the occlusion while the guidewire shown in FIGS. 9A and 9B crosses the occlusion, such as described above.

In another embodiment, after the guidewire has cleared some space, the guidewire is exchanged for a guidewire having an occlusive device as described above. Further details regarding this type of exchange are described in U.S. Pat. No. 6,159,195, the entirety of which is hereby incorporated by reference. In addition, if an aspiration catheter is already provided on the guidewire, the aspiration catheter itself may be used for the exchange.

Once the guidewire having an occlusive device is delivered, the vessel is then treated such as described above. For instance, an aspiration catheter may be used as described above to deliver drugs to dissolve the occlusion, followed by aspiration. These procedures preferably occur while the balloon on the catheter is inflated. The aspiration catheter is then removed and, optionally, the therapy catheter is inserted to perform therapy, the therapy catheter is removed and the aspiration catheter is delivered to aspirate the particles resulting from the therapy.

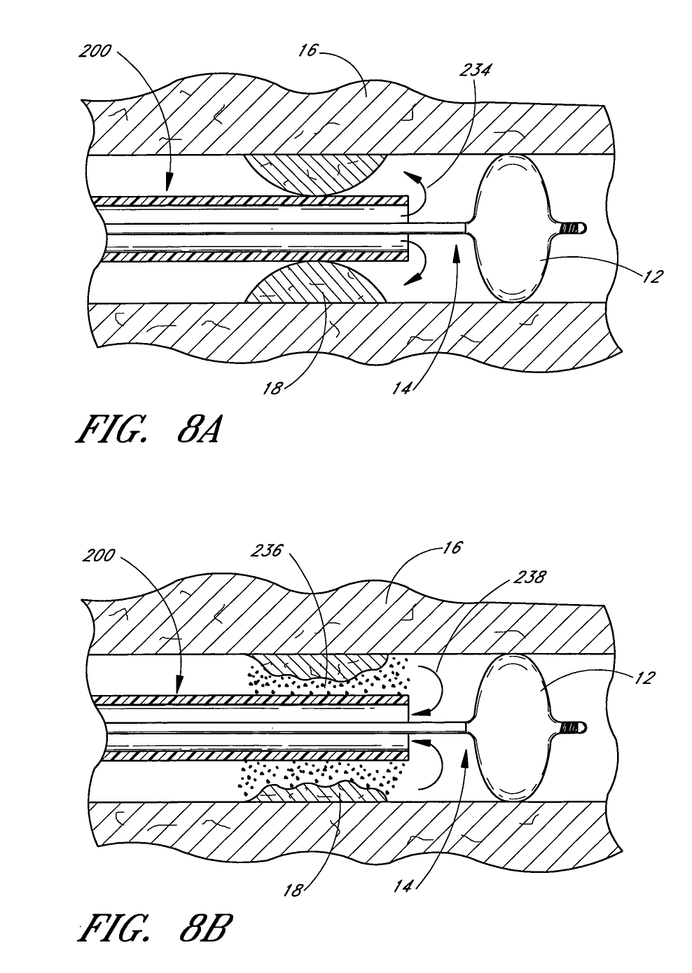

In another embodiment shown in FIG. 10A, a temporary occlusion balloon catheter 14 is delivered which contains irrigation holes 240 proximal to the balloon 12. These holes allow for the ejection of drugs to dissolve the occlusion 18 as the guidewire passes therethrough. Additionally, the same fluid used for drug delivery may also be the fluid used for balloon inflation. Drugs may be delivered both while the guidewire crosses the occlusion, and also while the balloon 12 on the guidewire 14 is inflated, shown in FIG. 10B. An aspiration catheter as described above may be used to aspirate particles while the guidewire crosses the occlusion and also while the balloon is inflated and drugs are delivered through the holes 240 in the guidewire

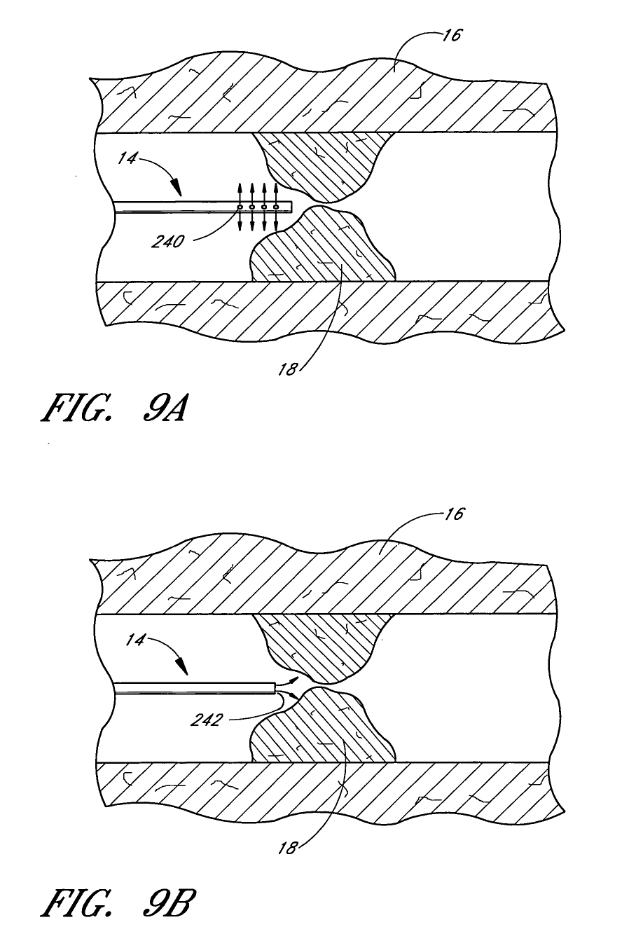

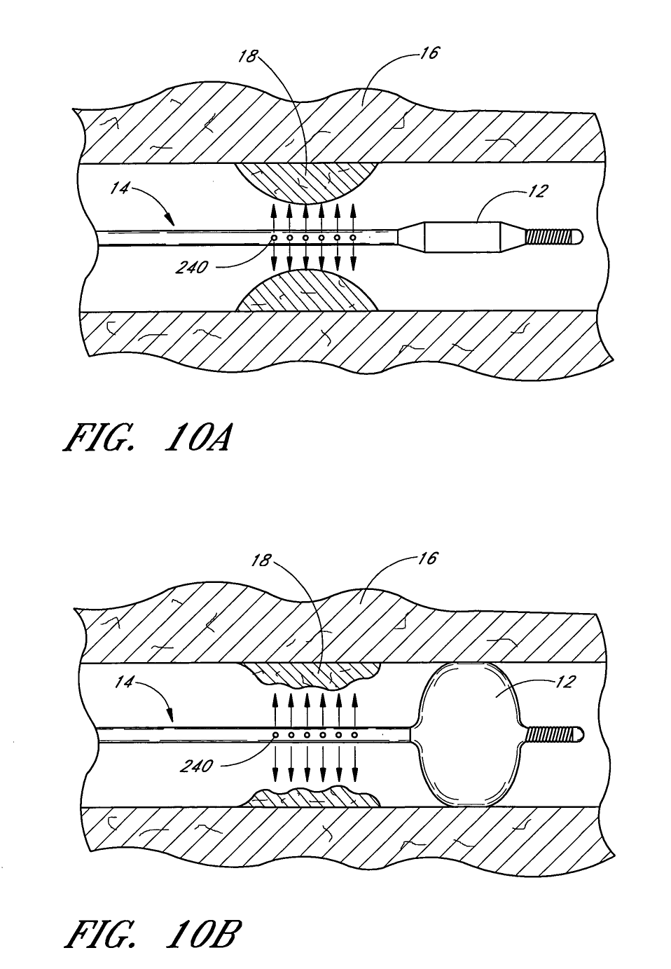

FIGS. 11A-11C illustrate other irrigation catheters for use with the above embodiments that provide a nitinol hollow guidewire having the capability to pass fluid therethrough. FIG. 11A illustrates a preferred embodiment of an irrigation catheter 302A constructed from a superelastic nitinol hollow wire. In this embodiment, the irrigation catheter 302A is comprised of an hypotube 304 and a coil member 306. The hypotube 304 is provided with proximal and distal ends 308 and 310 as well as a lumen 312 extending along the hypotube 304, thereby providing a fluid passageway. The coil member 306 of the catheter 302A is joined to the distal end 310 of the hypotube 304 as in the manner shown in FIG. 11A. The distal end 310 of the hypotube 304 may also include one or more perforations 314 thereof so that fluids can be delivered into or received from the desired body locations. In addition to distal perforations 314, gaps between the coil turns 316 also provide an effective passageway to deliver or receive fluids through coil member 306. Therefore, in this embodiment, perforations 314 at the distal end 310 of the hypotube 304 are optional so that the fluid may exit or enter the catheter 302A from the coil member 306. Although the catheter 302A of this embodiment can be used for delivering drugs to the distal body locations, the catheter 302A can also be used in those applications where irrigation and aspiration are necessary for emboli removal. For the most available cardiovascular catheters, the outer diameter of this irrigation catheter is about 0.38″ or smaller.

FIG. 11B shows a second embodiment which comprises a multilumen irrigation catheter 302B. In this embodiment, a portion of the catheter 302B comprising the hypotube 304 and the coil member 306 is configured similar to that of first embodiment. As a departure from the previous embodiment, however, the present embodiment also comprises a balloon member 318 and a conduit 320. The conduit 320 is preferably disposed along the inner lumen 312 of the hypotube 304. The balloon member 318 is coaxially mounted on the distal end 310 of the hypotube 304 as in the manner shown in FIG. 11B. The conduit 320 is provided with distal and proximal ends 322 and 324 as well as an inner lumen 326.

In this embodiment, the proximal end 322 of the conduit 320 is preferably connected to a gas source (not shown), while the distal end 324 is connected to the balloon member 318 through an inlet port 328 in the distal end 310 of the hypotube 304. The distal end 324 of the conduit 320 and the inlet port 328 are sealably connected to each other by suitable means such as adhesive to avoid any gas leak. In this arrangement, the inner lumen 326 of the conduit 320 connects the gas source to the balloon member 318 so that the gas from the gas source can inflate the balloon member 318.

The conduit 320 is preferably made of a flexible material such as polyimide, polyamide, or the like alloy and is in the form of hypotubing. Preferably, the outer diameter of the conduit 320 is significantly smaller than the inner diameter of the lumen 312 of the hypotube 304 so that fluid in the lumen 312 can flow without any restriction. In this embodiment, carbon dioxide (CO₂) gas is preferably employed to inflate balloon member 318. In fact, (CO₂) gas easily dissolves in blood and does not cause any harm in the patient's body, if an accidental leak occurs. If desired, however, the balloon member may be inflated using any of a number of harmless gases or fluids, or possible combinations thereof. In applications, the irrigation catheter 302B may function as the catheter 302A in the first embodiment. However, with the inflatable balloon member 318, the catheter 302B can be advantageously used for occlusion and irrigation therapies.

FIG. 11C shows a third embodiment which comprises another single lumen catheter 302C as in the case of first embodiment. In this embodiment, a portion of the catheter 302C comprising the hypotube 304 and the coil member 306 is also configured similar to that of first embodiment. The present embodiment also comprises a balloon member 318. The balloon member 318 is coaxially mounted on the distal end 310 of the hypotube 304 as in the manner shown in FIG. 11B. Fill holes 330 are provided in the wall of the distal end 304 of the hypotube 304 along the section of hypotube enclosed within the balloon member 318. During the application, these fill holes 330 allow the passage of irrigation fluid into the balloon member 318. As the fluid pressure reaches up to inflation pressure of the balloon member 318, the balloon member is inflated. An exemplary inflation pressure range for the occlusion balloons can be given as 40 psi. However, for the therapeutic balloons, such pressure range can be as high as 200 psi.

As shown in FIG. 11C, a number of valve members are also provided over the inner wall of the distal end 310 of the hypotube 304. The valve members are attached over the perforations 85 as in the manner shown in FIG. 11C. Preferably, the valve members 332 are comprised of elastomeric membranes. These membranes 332 can be configured and dimensioned to withstand some threshold fluid pressure, such as the inflation pressure of the balloon member 318.

In application, any pressure over this threshold pressure breaks open these membranes 332, i.e., activates valves 332, and delivers the irrigation fluid, through perforations 314, into the body locations. The fluid delivery can be also provided through leakages from both optional slits (not shown) in the balloon member 318 and the gaps between the coil turns 316. As in the previous embodiment, the catheter 302C can be advantageously used for occlusion and irrigation therapies.

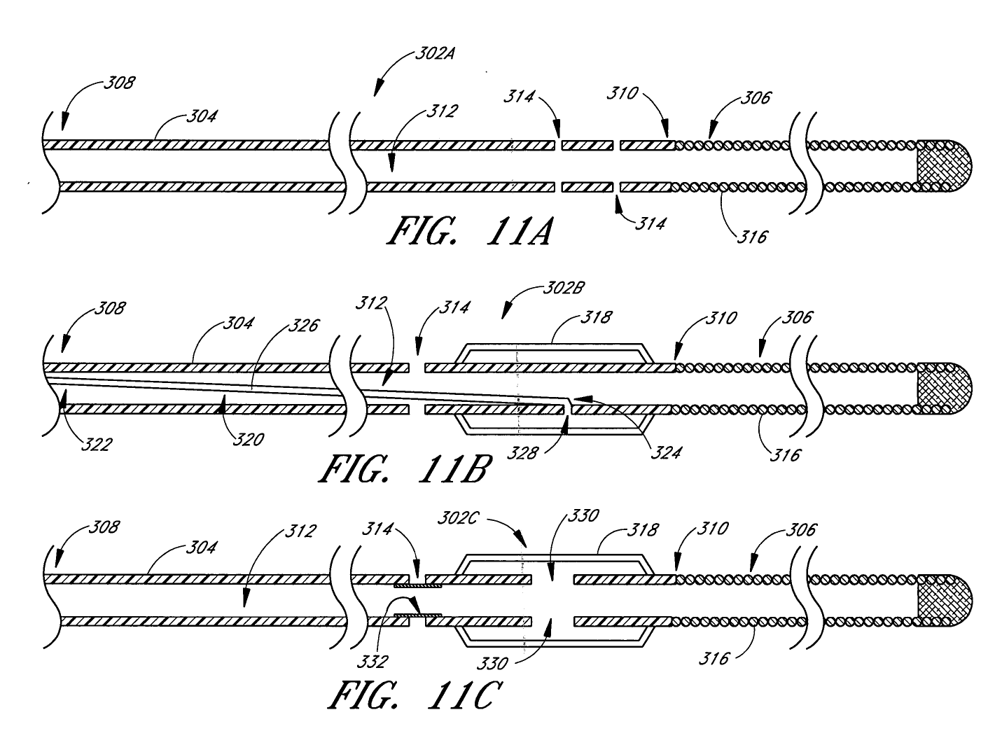

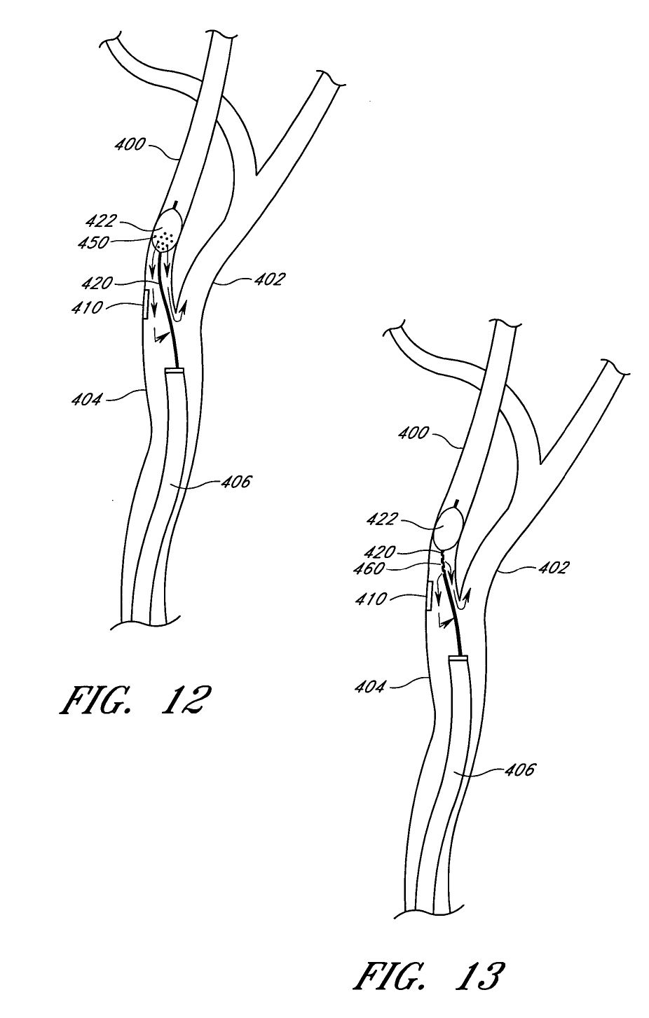

FIG. 12 shows another embodiment of an occlusive device capable of passing saline solution, drugs or other fluids across an occlusion. Although this occlusive device is shown for treatment in the carotid arteries, it will be appreciated that the device and method may be used in other locations as well.

A main catheter 406, with or without a distal occlusive device, is introduced into the patient's vasculature through an incision in the femoral artery in the groin of the patient or through direct access to the arteries in the neck. The main catheter 406 is guided through the vasculature until it reaches the common carotid artery 404, where it can remain in place throughout the procedure.

Once the main catheter 406 is in place proximal to the occlusion 410, an inner catheter or guidewire 420 having an occlusive device 422 at its distal end is delivered through the main catheter 406 into the internal carotid artery 400 and past the site of the occlusion 410. Alternatively, a detachable occlusive device can be deployed at the site distal to the occlusion, and the delivery device removed. In this example, the occlusive device 422 is an inflatable balloon. The balloon is inflated to occlude the internal carotid artery 400 at a site distal to the occlusion 410. It should be understood that the occlusion within the artery can be in a discrete location or diffused within the vessel. Therefore, although placement of the distal occlusive device is said to be distal to the occlusion to be treated, portions of the diffuse occlusion may remain distal to the occlusive device.

The occlusive device 422 preferably may be used to flush fluid across the occlusion 410. In one embodiment, the fluid may be saline solution or another suitable flushing solution. In another embodiment, the fluid may be any one of a number of drugs such as described above. The fluid may be advantageously passed through a lumen in the guidewire 420 and into the occlusive device 422. The occlusive device 422 has at least one fluid flow opening and is preferably microporous on its proximal end, having a plurality of holes 450 (e.g., 10-50) that are preferably less than 1000 microns in diameter and more preferably between 50 and 100 microns in diameter. The holes may be formed in the occlusive device 422 by laser drilling, for example. As fluid passes through the occlusive device 422 and into the internal carotid 400, emboli, particulates, and other debris are flushed past the treated occlusion 410 and down the external carotid 402. In embodiments where the occlusion is not formed near the branching of two vessels, the fluid may be isolated across the occlusion as it flows in a proximal direction away from the balloon. Thus, when the fluid used is a drug as described above, the drug is preferably localized across the occlusion for treatment.

Fluid flow may be maintained with a pressurized syringe or other suitable inflation device, as described above, located outside the patient. The fluid is used for inflating the occlusive device 422 as well as for irrigating emboli from the internal carotid 400 down the external carotid 402, or for localizing drugs across the occlusion.

Another irrigation device and method is disclosed in FIG. 13, in which one or more holes 460 in the guidewire 420 are located distal to the treated lesion 410 and proximal to the occlusive device 422. (For example, 1, 2, or 3 holes of dimensions 0.050″×0.002-0.003″ may be used, or 10 holes of dimensions 0.003″×0.003″, to provide a flow such that the pressure inside the vessel does not exceed 50 psi.) Fluid is pumped through the guidewire 420 and out of the holes 460 (which may advantageously be 50-300 microns in diameter) to flush away emboli from the treated lesion 410N and down the external carotid 402, or to localize drugs to a desired treatment location. The guidewire 420 may have a single lumen (not shown) that is in fluid communication with both the internal carotid artery 400 (via the holes 460) and the occlusive device 422, in which case the irrigation fluid and the fluid used to inflate the occlusive device 422 are the same. Alternatively, the guidewire 420 may have dedicated lumens (not shown) for irrigation and inflation..

Instead of pumping irrigation fluid through the holes 460 as shown in FIG. 13, a larger slot (not shown) of dimensions 0.005″×0.100-0.200″ may be cut into the guidewire 420 and then covered with a braid (not shown) that extends 0.010-0.030″ beyond the edges of the slot. As irrigation fluid is passed through the guidewire 420, the braid expands, permitting the irrigation fluid to pass out of the slot and into the internal carotid 400. Instead of using a braid, this slot may alternatively be covered with a plastic sheath (not shown) having a plurality of slits or pores (not shown) which are in fluid communication with the slot. Ten pores having a diameter of 50-100 microns may advantageously be used.

Fluid flow rates for the methods disclosed in FIGS. 12 and 13 are preferably between about 0.1 cc/sec and 10 cc/sec, more preferably about 0.1 cc/sec and 3 cc/sec, more preferably between about 0.5 and 1.5 cc/sec, and still more preferably about 1 cc/sec. The fluid pressure may be pulsed on and off to better flush away emboli or treat the occlusion. For example, fluid pressure may be alternately applied for 5 seconds (in the form a pulse) and then turned off for 2-3 seconds.

Further details regarding the devices of FIGS. 12 and 13 and other devices and methods are described in Applicant's copending application entitled METHOD FOR CONTAINING AND REMOVING OCCLUSIONS IN THE CAROTID ARTERIES, Ser. No. 09/270,150, filed Mar. 16, 1999, now abandoned, the entirety of which is hereby incorporated by reference.

The preferred methods of the invention can be used especially following myocardial infarction, for totally occluded vessels and partially occluded vessels defined by TIMI 0-1 flow, and having no major side branch. However, the method is not intended to be limited only to such applications, and may also be used for vessels having blood flow through side branches. TIMI stands for “thrombolysis in myocardial infarction.” This value is measured angiographically by injecting a dye and noting the time it takes to clear through the blood vessel. A TIMI of 3 means that the vessel is open. A TIMI of 0 means that the vessel is totally occluded. In a totally occluded vessel, one cannot visualize past the site of the occlusion because the dye will not flow past the occlusion. Because the site cannot be visualized, a distal occlusive device generally cannot be used unless the occlusion is dissolved using methods such as described above.

D. Treatment Methods Described in U.S. application Ser. No. 08/650,464

In general, the catheter apparatus is for treatment of a stenosis in a lumen in a blood carrying vessel. It is comprised of a main catheter and a balloon-on-a-wire device. The main catheter is comprised of a first flexible elongate tubular member having proximal and distal extremities. A first inflatable elastic balloon having an interior is coaxially mounted on the distal extremity of the first flexible elongate tubular member. The first flexible elongate tubular member has a balloon inflation lumen therein in communication with the interior of the first balloon. The first elongate tubular member has a main lumen therein extending from the proximal extremity to the distal extremity and exiting through the distal extremity. An adapter is mounted on the proximal extremity of the first flexible elongate tubular member and has a balloon inflation port in communication with the balloon inflation lumen, a therapeutic catheter port and an aspiration port in communication with the main lumen. The balloon-on-a-wire device is comprised of a guide wire having proximal and distal extremities.

A second inflatable elastic balloon has an interior and is coaxially mounted on the distal extremity of the guide wire. The guide wire has a balloon inflation lumen therein in communication with the interior of the second balloon. The balloon-on-a-wire device is slidably mounted in the therapeutic catheter port and in the main lumen of the first elongate tubular member with the proximal extremity of the guide wire being disposed outside of the main lumen. Removable valve means is carried by the proximal extremity of the guide wire and has the capability of forming a fluid-tight seal with respect to the guide wire while permitting relative axial movement of the guide wire and the first flexible elongate tubular member with respect to each other whereby the first balloon can be moved so that it is proximal of the stenosis and the second balloon so that it is distal of the stenosis. The removable valve means includes an inflation port in communication with the balloon inflation lumen and the guide wire. The apparatus is also comprised of means coupled to the balloon inflation port of the first flexible elongate tubular member for inflating the first balloon and means coupled to the balloon inflation port of the removable valve means for inflating the second balloon to create a working space which brackets the stenosis.

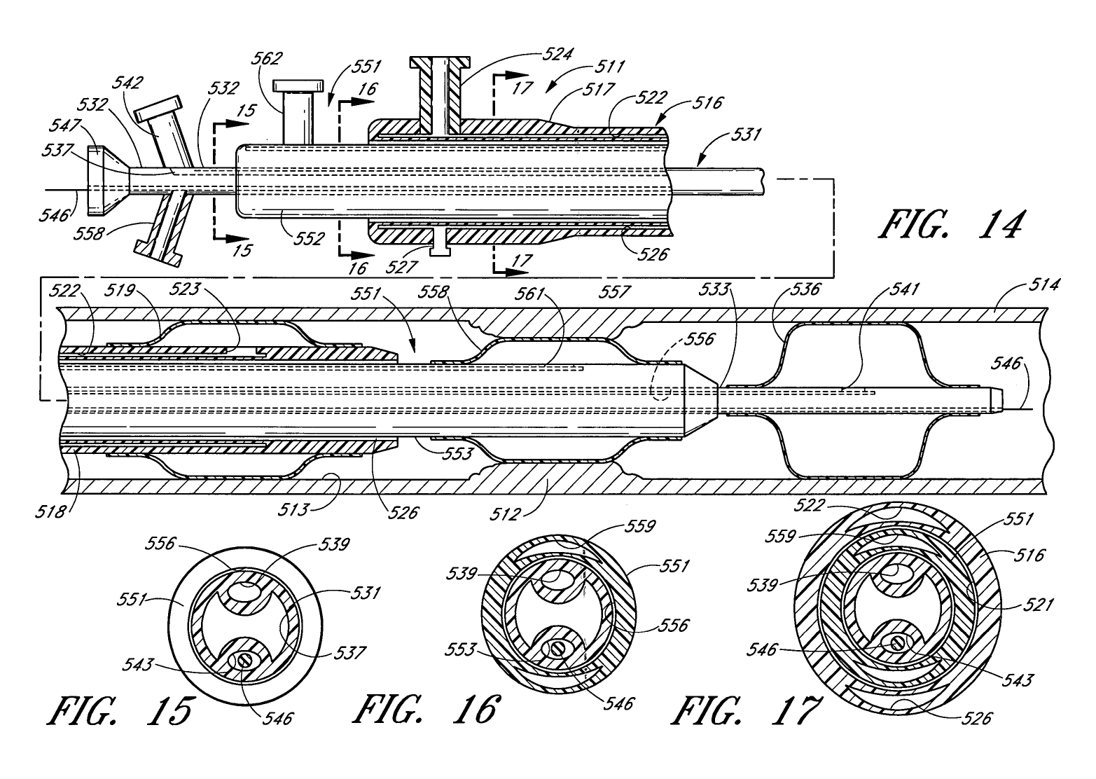

More particularly as shown in FIGS. 14-17, the catheter apparatus 511 of the present invention is for use in the treatment of a stenosis 512 in a lumen 513 in a blood-carrying vessel 514 in which the stenosis 512 has a length and a width or thickness which at least partially occludes the lumen 513. The apparatus consists of a first elongate flexible tubular member 516 formed of a suitable plastic material which is provided with proximal and distal extremities 517 and 518. A first balloon 519 is mounted on the distal extremity 518 and preferably is a compliant balloon formed of a suitable elastic material such as a latex or a very low radiation polyethylene so that it can be inflated to the size of the vessel 514 in which it is to be disposed. Thus, the balloon 519 should be capable of expanding to various diameters depending on the size of the vessel. The first balloon 519 can be formed as a separate balloon separate from the elongate tubular member 516 as shown and adhered thereto by suitable means such as an adhesive (not shown), or it can be formed integral with the tubular member 516 in a manner well known to those skilled in the art.

The tubular member 516 is provided with a large centrally disposed or main lumen 521 extending from the proximal extremity 517 to the distal extremity 518. It is also provided with a balloon inflation lumen 522 which has a distal extremity in communication with the interior of the first balloon 519 through a port 523. The proximal extremity of the balloon inflation lumen 522 is in communication with a balloon inflation fitting 524 mounted on the proximal extremity 517 of the tubular member 516. The fitting 524 can be of a conventional type as for example a Luer-type fitting which is adapted to be connected to a balloon inflation device (not shown) for inflating and deflating the first balloon 519.

The first tubular member 516 is also provided with an aspiration lumen 526 which exits through the distal extremity 518 and the proximal extremity 517 of the tubular member 516. A Luer-type fitting 527 is mounted on the proximal extremity 517 and is in communication with the aspiration lumen 526. The fitting 527 is adapted to be connected to a suitable aspiration or suction source (not shown) of a conventional type such as a syringe or rubber bulb for aspiration purposes as hereinafter described.

The catheter assembly or apparatus 511 also consists of a second elongate flexible tubular member 531 having proximal and distal extremities 532 and 533. A second inflatable balloon 536 of the same type as the first inflatable balloon is coaxially mounted on the distal extremity 533 in a conventional manner. The tubular member 531 is provided with a large generally centrally disposed arterial blood flow lumen 537 which opens through the distal extremity 533 and is in communication with a Luer-type fitting 538 which as hereinafter described is adapted to be connected to a supply of arterial blood from the patient which for example can be taken from another femoral artery of the patient by the use of a blood pump.

The second tubular member 531 is also provided with a balloon inflation lumen 539 which is in communication with the interior of the second inflatable balloon 536 through a port 541. The proximal extremity of the lumen 539 is in communication with the Luer-type fitting 542 mounted on the proximal extremity 532 of the second tubular member 531 and as with the balloon inflation fitting 524 is adapted to be connected to a balloon inflation-deflation device (not shown) of a conventional type. The second tubular member 531 is also provided with a lumen 543 which also can be used as a guide wire and/or for introducing a saline solution extending from the proximal extremity to the distal extremity. The lumen 543 is sized so that it is adapted to receive a conventional guide wire 546 as for example a 0.014″ or 0.018″ guide wire and extends from the proximal extremity to the distal extremity so that the guide wire 546 can extend beyond the distal extremity of the second tubular member 531. A fitting 547 is provided on the proximal extremity 532 in communication with the lumen 543 for introducing the saline solution.

As shown in FIG. 14, the second tubular member 531 is disposed within the central lumen 521 of the first tubular member 516 and is slidably and coaxially mounted therein for displacement of the second balloon 536 with respect to the first balloon 519 as hereinafter described.

The catheter assembly or apparatus 511 also consists of a third elongate flexible tubular member 551 having proximal and distal extremities 552 and 553. It is provided with a centrally disposed lumen 556 extending from the proximal extremity 552 to the distal extremity 553 and through which the second tubular member 531 is coaxially and slidably mounted.

Means 557 is provided on the distal extremity 553 of the third tubular member 551 for performing a medical procedure. In the embodiment of the invention shown in FIG. 14, this means 557 consists of a third balloon 558 which can be non-compliant coaxially mounted on the distal extremity of the third tubular member 551. The third balloon 558 can be attached in the same manner as the first and second balloons 519 and 536 hereinbefore described. The third tubular member 551 is provided with a balloon inflation lumen 559 which has its distal extremity in communication with the interior of the balloon 558 through a port 561. The proximal extremity of the balloon inflation 559 is in communication with a Luer-type fitting 562 provided on the proximal extremity 552 and adapted to be connected to a conventional inflation deflation device (not shown) for inflating and deflating the third balloon 558.

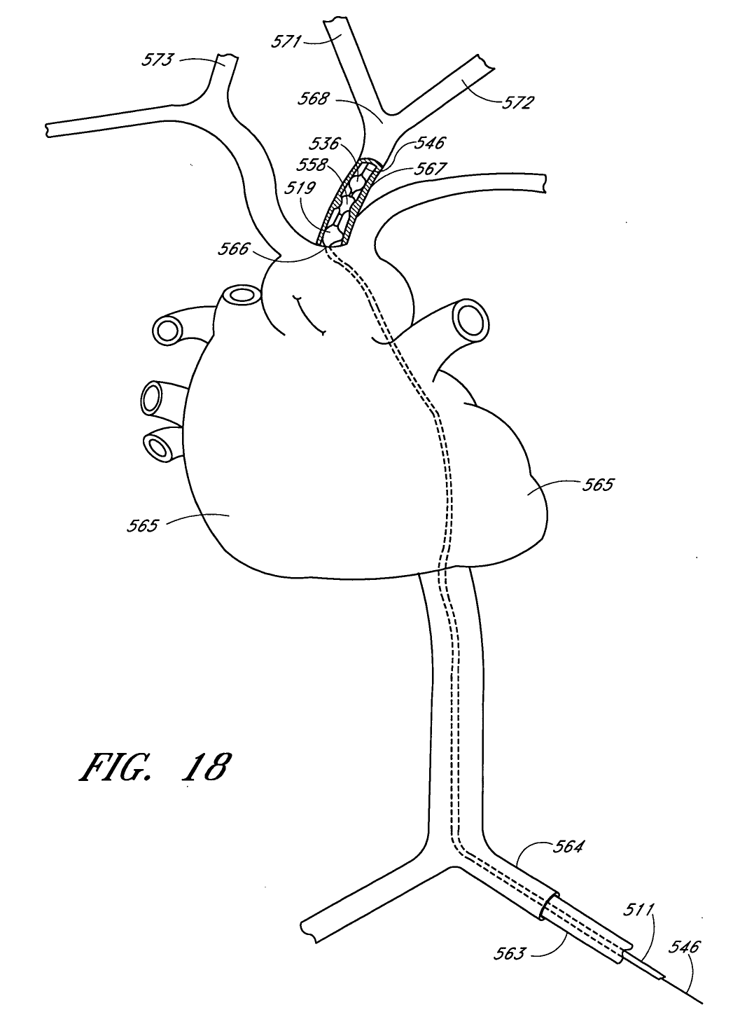

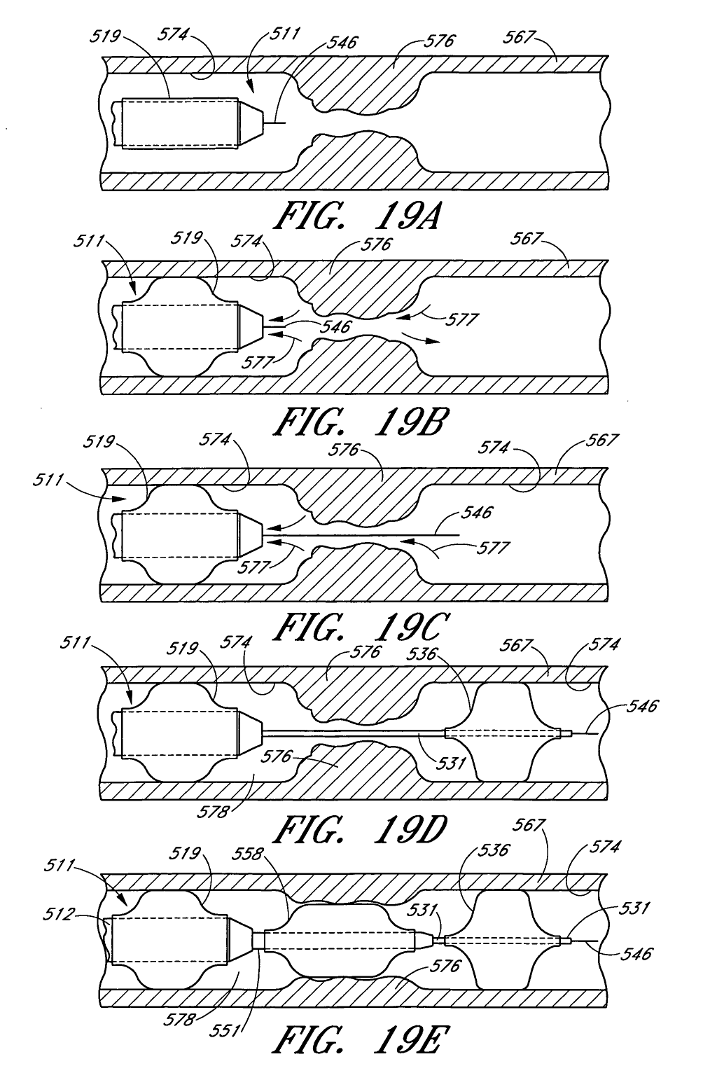

The operation and use of the catheter assembly or apparatus 511 in the method of the present invention for treating occluded vessels may now be briefly described in connection with an occlusion formed by a stenosis in a vessel not having a bifurcation therein as for example in saphenous graft or in one of the right and left carotid arteries, also called internal and external carotid arteries, of a patient in connection with the illustrations shown in FIGS. 18 and 19A-19E. A guiding catheter 563 (FIG. 18) of a conventional type is inserted into an incision into a femoral artery 564 of a patient and is advanced through that artery into the aorta of the heart 565 of the patient and into the ostium 566 of the selected carotid artery or vessel as for example the left carotid 567.

After the guiding catheter has been appropriately positioned, the guide wire 546 is introduced separately into the guiding catheter or along with the catheter assembly 511. The distal extremity of the catheter apparatus or assembly 511 with all of the first, second and third balloons 519, 536 and 558 completely deflated, is introduced into the guiding catheter 563 along with or over the guide wire 546 and is advanced through the guiding catheter 563 into the ostium 566 of the carotid artery or vessel 567 and into the lumen or passageway 568 of the vessel as shown in FIGS. 18 and 19B.

The distal extremity of the catheter assembly 511 is advanced until it is just proximal of a stenosis 569 in the carotid artery 567 to be treated. The balloon 519 is then inflated by introducing a suitable inflation medium such as a radiopaque liquid into the fitting 524 to cause it to pass through the balloon inflation lumen 522 through the port 523 and into the interior of the first balloon 519 to inflate the same as shown in FIG. 19B. The balloon 519 is progressively inflated until it engages the side wall of the vessel 567 to occlude the vessel 567. At the time that this is occurring, a negative pressure or suction is applied to the aspiration fitting 527 to supply a negative pressure through the balloon inflation lumen 522 to suck or aspirate blood in the vessel 567 distal of the first balloon 519 into the aspiration lumen 526 and out the aspiration fitting 527 to thereby reverse the flow of blood through the stenosis as shown by the arrows 571 in FIG. 19B.

While a reverse flow of blood is occurring in the vessel 567, the guide wire 546 is advanced through the stenosis 569 as shown in FIG. 19C. In the event that any pieces or particles of plaque are knocked off of the occlusion formed by the stenosis 569 by movement of the guide wire 546 through the same, such pieces of plaque or emboli will be drawn out with the reverse flow of blood into the aspiration lumen 526 and out of the aspiration fitting 527. During the time that the guide wire 546 is being advanced through the stenosis 569 it may be desirable at the same time to introduce a saline solution through the guide wire lumen 543 of the second elongate flexible tubular member 531 to exit through the distal extremity of the second elongate flexible tubular member 531 into the space immediately proximal of the stenosis 569. This introduced saline solution aids the flow of particulate or other particles dislodged from the stenosis 569 during advancement of the guide wire 546 through the same and carries them back with the mixed saline blood solution through the aspiration lumen 526 in a manner hereinbefore described.

With the guide wire 546 remaining in position, the second elongate flexible tubular member 531 with the second balloon 536 thereon in a deflated condition is advanced over the guide wire 546 through the stenosis 569 until the second balloon 536 is distal of the stenosis 569 as shown in FIG. 19D after which the second balloon 536 is inflated by introducing an inflation medium as for example a radiopaque liquid through the inflation fitting 542 into the lumen 539 through the port 541 to the interior of the second balloon 536 to inflate the second balloon 536 until it engages the sidewall of the vessel 567.

Prior to, during or after inflation of the second balloon 536, the guide wire 546 can be removed. However, it is preferable to remove the guide wire 546 as soon as the second balloon 536 has been advanced so that it is beyond the stenosis 569. At this time, and certainly prior to complete inflation of the second balloon 536, blood is shunted across the stenosis 569 and into the lumen 568 distal of the second balloon 536 by introducing blood through the fitting 538 and into the centrally disposed blood flow lumen 537 in the second tubular member 531 so that it exits out the central lumen 537 distal of the second balloon 536. The blood which is supplied to the fitting 537 can be taken from another femoral artery of the patient and pumped into the fitting 538. In addition, if desired, the blood which is aspirated in the space distal of the first balloon 519 can be appropriately filtered and also supplied to the fitting 538. By shunting blood past the stenosis 569 in this manner it can be seen that blood is being continuously supplied to the carotid artery of the patient during the time that the second balloon 536 is inflated and occludes the lumen 568 in the vessel 567.

As soon as the second balloon 536 has been inflated, it can be seen that there is provided a working space 576 (FIG. 19D) between the first and second balloons 519 and 536 so that medical procedures can be undertaken to remove or reduce the stenosis 569 in the space between the first and second balloons 519 and 536.

Assuming that it is desired to compress the plaque or material forming the stenosis 569 to provide a larger lumen, opening or passageway through the stenosis 569 the third tubular member 551 can be advanced by grasping the proximal extremity 552 to cause the distal extremity with the third balloon 558 thereon to be advanced into the working space 576. As soon as the balloon 558 has been properly positioned within the stenosis 569, the balloon 558 also can be inflated with a suitable inflation medium as for example a radiopaque liquid. The balloon 558 can be inflated to the desired pressure to cause compression of the plaque of the occlusion against the sidewall of the vessel 567 by the application of appropriate pressure. As in conventional angioplasty procedures, the third balloon 558 can be formed of a non-elastic relatively non-compliant material so that high pressures as for example 10-15 atmospheres can be used within the balloon to apply compressive forces to the vessel without danger of rupturing the vessel. It should be appreciated that the non-elastic capabilities can also be achieved by a composite elastic material.

Since the blood flow has been restored to the vessel 567 by the shunt hereinbefore described, the compression of the occlusion forming the stenosis 569 can be carried out for an extended period of time, as for example after a few minutes, if desired to help ensure that a large lumen or passageway is formed through the stenosis 569 as shown in FIG. 19E. If it is believed that the occlusion forming the stenosis 569 has been sufficiently compressed, the third balloon 558 can be deflated. In the event an inelastic balloon is utilized for the third balloon 558, and it is desired to utilize a larger third balloon, this can be accomplished by removing the third tubular member 551 with the deflated balloon 558 thereon and introducing a third tubular member 551 having a larger size balloon thereon over the second tubular member 531 and advancing it into the stenosis 569 and inflating the larger size balloon to create a still larger passage through the stenosis 569.

After the appropriate dilation the stenosis 569 has been accomplished the third balloon can be removed from the stenosis while aspiration of the working space 576 is still ongoing so that any plaque coming off the occlusion forming the stenosis 569 can be aspirated out of the vessel. After the third balloon 558 has been removed from the stenosis, the second balloon 536 and the first balloon 519 can be deflated to permit normal blood flow through the vessel 567 after which the arterial blood flow supply to the fitting 538 can be terminated. The entire catheter assembly 511 can then be removed from the guiding catheter 563 after which the guiding catheter 563 can be removed and a suture applied to the incision created to obtain access to the femoral artery.

In place of the third balloon 558 for causing compression of the occlusion forming the stenosis 567 to create a larger passageway therethrough, an atherectomy device 581 (see FIG. 20) can be utilized for operating in the working space 576 to remove the plaque of the occlusion forming the stenosis. This can be accomplished with a catheter assembly or apparatus 581 which in many respects is similar to the apparatus 511 shown in FIG. 14 and consists of a first tubular member 516 with a first balloon 519 and a second tubular 531 with a second balloon 536 thereon. In place of the third flexible elongate tubular member 551 there is provided a third flexible elongate tubular member 586 which is provided with proximal and distal extremities 587 and 588. The flexible elongate tubular member 586 is slidably and rotatably mounted in the central lumen 521 of the flexible elongate member 516 and is provided with a central or main lumen 589 through which the second flexible elongate tubular member 531 extends. It is also provided with a lumen 591 extending from the proximal extremity to the distal extremity through which a saline solution can be introduced for saline irrigation as hereinafter described. It is also provided with another lumen 592 which is adapted to receive a plurality of electrical conductors 593 for performing electrical functions as hereinafter described. The lumen 592 is connected to a conventional Luer-type fitting 596 serving as a fluid irrigation fitting mounted on the proximal extremity first tubular member 512 and is in communication with an annular recess 597 which is in communication with the lumen 591 provided in the tubular member 586 for supplying a saline irrigation liquid through the flexible elongate tubular member 586 and into the working space 576 provided between the first and second balloons 516 and 536. In order to aid aspiration of the saline irrigation liquid from the working space 576, the outer surface of the flexible elongate tubular member 586 is provided with a helical groove 598 therein which has one end which opens into the working space 576 and which has the other end in communication with the aspiration fitting 527.

Means is provided for rotating the second tubular member 586 and consists of suitable means such as a spur gear 601 mounted on the proximal extremity 587 of the tubular member 586. The spur gear 601 is driven in a suitable manner as for example by another smaller spur gear 601 which is of greater width than spur gear 601 so as to provide a splined gear connection between the gears 601 and 602. This accommodates the desired longitudinal movement for the tubular member 586 so that the distal extremity 588 of the tubular member 586 can be advanced and retracted in the working space 576 as hereinbefore described. An electrical drive motor 603 is provided for driving the gear 602.

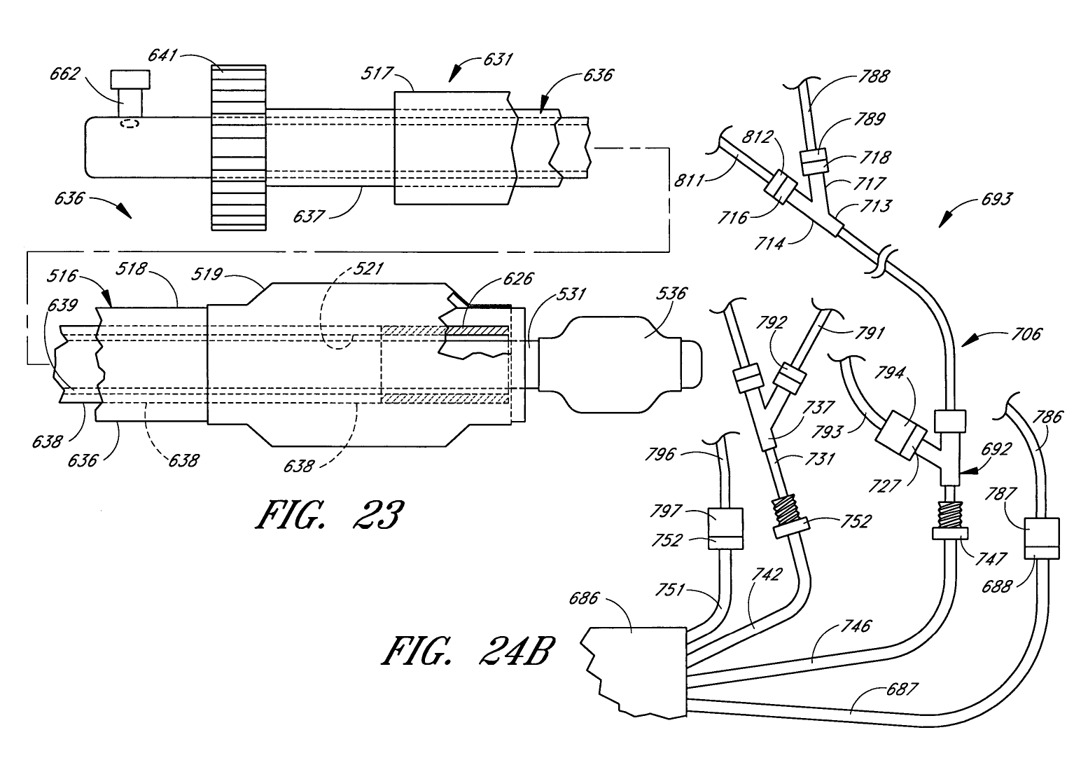

Atherectomy means 606 is provided on the distal extremity 588 of the flexible elongate tubular member 586. As shown in FIGS. 20 and 22, the atherectomy means 606 consists of a flexible elongate member 607 formed of a suitable material such as stainless steel or preferably a superelastic Nitinol. The flexible elongate member 607 is wound into a helix as shown in FIG. 22 onto the distal extremity of the tubular member 586. The flexible elongate member 607 can be formed of a ribbon having a thickness of 0.003″ and a width of 0.060″. One end of the flexible elongate member 607 can be secured to the tubular member 586, as for example by inserting the same into a slit 608 and additionally by the use of adhesive (not shown). The flexible elongate member 607 is wrapped into a helix in a direction opposite to the direction of normal rotation of the tubular member 586 and can be provided with a special tip 609 on its free end with the tip having an arcuate surface 611 that is inclined rearwardly to terminate at a cutting edge 612 (see FIG. 22) which is adapted to engage the plaque or the stenosis 569.

When the distal extremity 588 of the flexible elongate tubular member 586 has been introduced into the working space 576, the end or tip 609 of the flexible elongate member 607 of the atherectomy means 606 is free. A saline solution is introduced into the fitting 557. Thereafter the motor 603 can be energized to cause rotation of the tubular member 586 and to thereby cause rotation of the helically wound flexible elongate member 607 to cause its free end or tip 609 to be moved outwardly radially under centrifugal force to bring the cutting edge 612 into engagement with the plaque 569 in the stenosis 569 to cause progressive removal of the plaque forming the stenosis 569 to enlarge the passageway extending through the stenosis. Because of the rounded configuration of the tip 609, the tip 609 will not dig into the vessel wall but will only remove plaque which is engaged by the cutting edge 612. As the plaque is being removed, the saline solution introduced through the fitting 596 into the space 576 picks up the plaque particles or emboli as they are being removed. The saline solution with the plaque or emboli therein is removed through the spiral groove 598 and through the aspiration port 527. The flexible elongate tubular member 586 can be moved back and forth so that the cutting tip 609 engages the length of the stenosis 569 so that substantially all of the stenosis 569 can be removed.

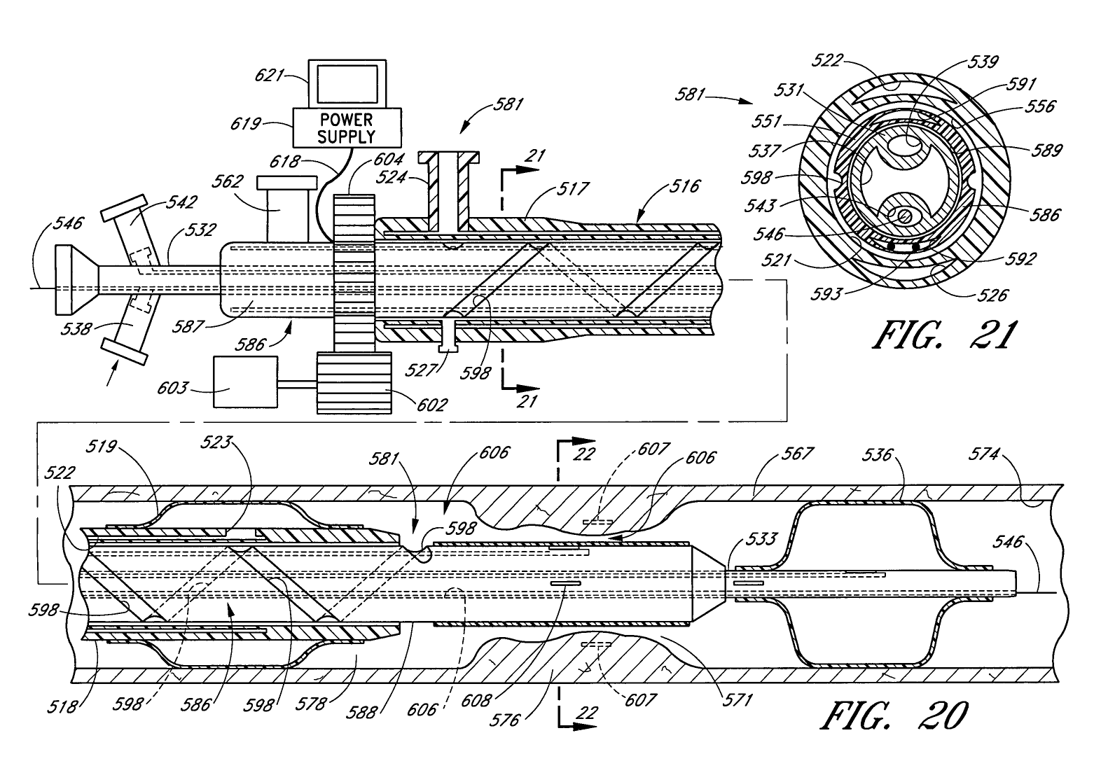

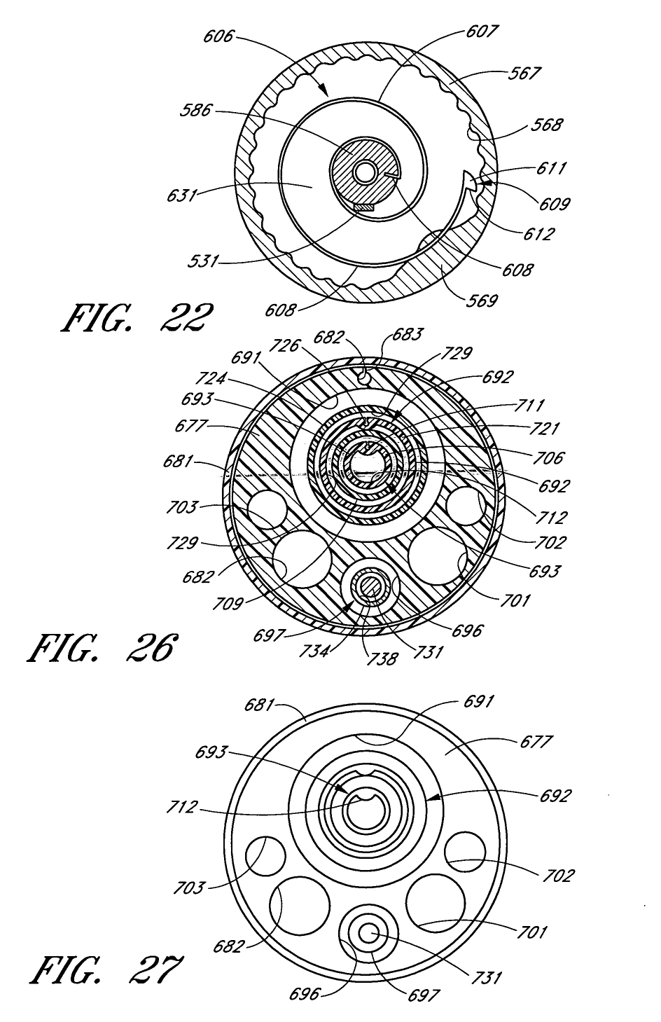

Means is provided to sense when sufficient plaque has been removed from the stenosis 569 and to ensure that cutting edge 612 does not cut into the vessel wall. An ultrasonic sensor 616 (see FIG. 20) is mounted in the distal extremity of the tubular member 586 and is connected by conductors 593 (see FIG. 21) extending through the lumen 592 and connected to a cable 618 which is connected to an ultrasonic power supply 619 and a video monitor 621. By using the Doppler effect, ultrasonic energy can be utilized in connection with the transducer 616 to ascertain the depth of cut being made by the flexible elongate member 607 as it is being rotated.

As soon as a desired amount of plaque has been removed from the stenosis 569 to provide the desired passage through the stenosis, rotation of the tubular member 586 is terminated after which the tubular member 586 can be withdrawn followed by deflation of the second balloon 536 and withdrawing it. Deflation of the first balloon 516 then occurs after which it is withdrawn from the vessel 567. Thereafter, the guiding catheter 563 can be removed and the incision closed as hereinbefore described.

In order to ensure that restenosis will not take place, it may be desirable to place a cylindrical stent 626 in the stenosis 569. Such a stent 626 can be a self-expanding stent formed of a suitable material such as a superelastic Nitinol and movable between unexpanded and expanded conditions. Such a stent 626 can be placed by a suitable catheter apparatus 631 of the type shown in FIG. 23. The stent 626 which is cylindrical in form is pushed over the proximal extremity of the second elongate flexible tubular member 531 into the main or central lumen 521 so that it is retained in the unexpanded position. It is then pushed forwardly toward the distal extremity of the first flexible elongate tubular member 516 by means of a flexible elongate tubular member 636 having proximal and distal extremities 637 and 638 and having a flow passage 639 extending from the proximal extremity 637 to the distal extremity 638. The proximal extremity 637 is provided with a knurled collar 641 which is adapted to be engaged by the hand to facilitate pushing of the flexible elongate tubular member 636 so that its distal extremity is in engagement with the stent 626. Thus, when desired the stent 626 may be discharged or dislodged from the distal extremity of the second tubular member 531 and pushed into the working space 576 created between the first balloon 519 and the second balloon 536.

After the stent 626 has been discharged out of the end of the first flexible elongate tubular member 516, the stent 626 will self expand toward its expanded condition until it is in engagement with the wall of the vessel in the vicinity of the occlusion forming the stenosis 569 to frictionally retain the stent in engagement with the vessel wall. As soon as the stent 626 is in engagement with the vessel wall, the second balloon 536 can be deflated as can the first balloon 519. The first deflated balloon 536 can then be withdrawn through the interior of the cylindrical stent 626. This can be followed by deflation of the first balloon 519 and the removal of the flexible elongate tubular member 516 with its first balloon 519 and the flexible tubular member 531 with its second balloon 536, along with the flexible elongate member 636 until the entire catheter assembly or apparatus 631 has been removed from the guiding catheter 563. Thereafter the guiding catheter 563 can be removed and the incision sutured as hereinbefore described.

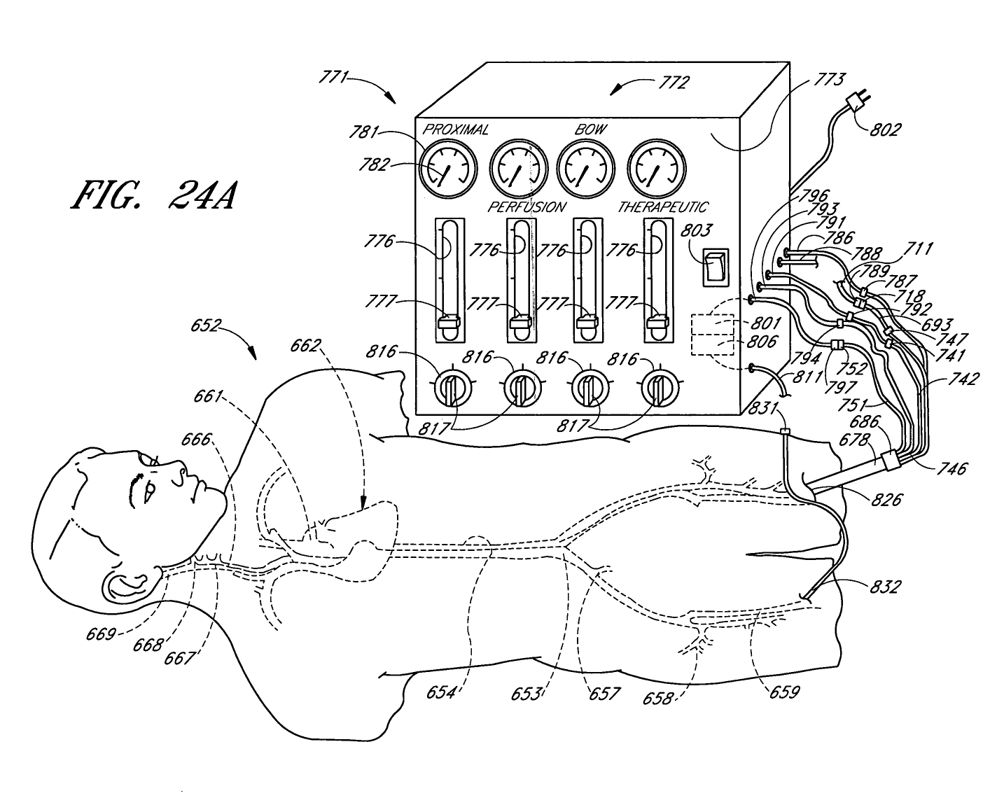

In FIG. 24, there is shown another embodiment of an apparatus 651 incorporating the present invention which is particularly adapted for use treating a stenosis at or near a bifurcation appearing in an arterial vessel. The apparatus 651 is shown being used on a human being 652 showing the principal arteries and pulmonary veins of the human body. Thus there as shown, the abdominal aorta 653 branches into the common iliac 654 which branches into the external iliac 656 and the internal iliac 657. The external iliac branches into the deep femoral artery 658 and into the femoral artery 659. The abdominal aorta 653 extending in the opposite direction passes through the aortic arch 661 of the heart 662. The aortic arch 661 is connected to the common carotid 666 which extends into a bifurcation 667 branching into the external carotid 668 and the internal carotid 669. Similar bifurcations appear in the basilar artery which is an artery which is particularly inaccessible for surgical treatment.

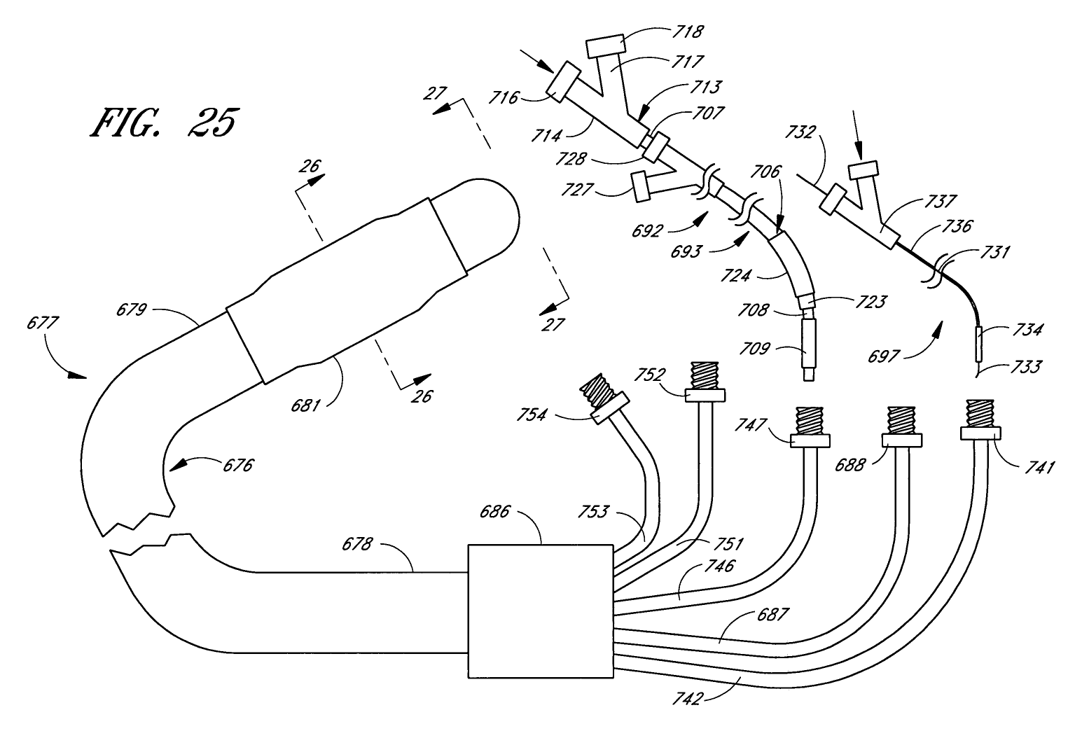

As hereinafter explained, the apparatus 651 shown in FIGS. 24, 25 and 26 consists of a proximal occlusion balloon catheter 676 which can be considered to be a first catheter. The catheter 676 is formed of a flexible elongate tubular member 677 having proximal and distal extremities 678and 679. The tubular member 677 is formed of a suitable material such as plastic and can have a suitable size ranging from 5 to 14 French and preferably 9 to 10 French. A balloon 681 is provided on the distal extremity 679 and is formed of a suitable elastic material. It is generally cylindrical in form and has its proximal and distal extremities secured to the tubular member 677 by suitable means such as an adhesive (not shown). The tubular member 677 is provided with a plurality of lumens therein. One lumen 682 serves as a balloon inflation lumen and extends from the proximal extremity 678. It can have a suitable size such as 0.024″ and has port 683 in communication with the interior of the balloon 681. A manifold 686 formed of a suitable material such as plastic is mounted on the proximal extremity 678. A tubular member 687 is mounted in the manifold 686 and is in communication with the inflation lumen 682.

The tubular member 677 is also provided with a large lumen 691 having a suitable size as for example 0.045″ which is adapted to slidably receive therein a therapeutic balloon catheter 692 and a perfusion balloon catheter 693. It is also provided with another lumen 696 having a suitable size as for example 0.026″ which is adapted to receive a balloon-on-a-wire catheter 697. It is also provided with an aspiration lumen 701 having a suitable size as for example 0.025″ and an irrigation lumen 702 having a suitable size as for example 0.015″. There is also provided another lumen 703 which can be used for other purposes.

The therapeutic balloon catheter 692 and the perfusion balloon catheter 693 are constructed in a manner similar to the balloon catheters hereinbefore described. Thus the perfusion balloon catheter 693 is provided with a flexible elongate tubular member 706 having proximal and distal extremities 707 and 708. A balloon 709 formed of an elastic material is secured to the distal extremity 708 by suitable means such as an adhesive (not shown) and is adapted to be inflated through a port 710 in communication with a balloon inflation lumen 711. The tubular member 706 is also provided with a blood perfusion lumen 712 which is centrally disposed therein. The proximal extremity 707 of the tubular member 706 is connected to a Y adapter or fitting 713 of which the central arm 714 is in communication with the blood perfusion lumen 712 and is provided with a Luer-type fitting 716. The side arm 717 of the fitting 713 is in communication with the balloon inflation lumen 711 and is provided with a Luer-type fitting 718 adapted to be connected to a source of pressure as hereinafter described.

The therapeutic balloon catheter 692 consists of a tubular member 721 having a proximal and distal extremities 722 and 723. A balloon 724 formed of a non-elastic material is secured to the distal extremity 723 by suitable means such as an adhesive. A port (not shown) is in communication with the interior of the balloon 724 and is in communication with a balloon inflation lumen 726. A Luer-type fitting 727 is mounted on the proximal extremity 722 and is in communication with the balloon inflation lumen 726. Another fitting 728 is mounted on the proximal extremity 722 and is in communication with a large centrally disposed lumen 729 which can receive the perfusion balloon catheter 693 for slidable movement as hereinafter described.

The balloon-on-a-wire catheter 697 is slidably mounted in the lumen 696 and consists of a guide wire 731 of a conventional construction having a suitable diameter as for example 0.018″ and having a proximal and distal extremities 732 and 733. A balloon 734 formed of a non-elastic material is mounted on the distal extremity 733 and is secured thereto by suitable means such as an adhesive (not shown). The proximal extremity of the balloon 734 is secured to the distal extremity of a tubular member 736 formed of a suitable material such as plastic and which is coaxially disposed on the guide wire 731. The tubular member 736 extends the length of the guide wire to the proximal extremity and is connected to a Luer-type wye fitting 737 and is in communication with an annular lumen 738 disposed between the tubular member 736 and the exterior surface of the guide wire 731. The lumen 738 is in communication with the interior of the balloon 734 for inflating and deflating the balloon 734. The balloon-on-a-wire catheter 697 is adapted to be introduced through a fitting 741 carried by a tube 742 mounted in the manifold 686 and in communication with the lumen 696 in the multi-lumen elongate tubular member 677.

A tube 746 is mounted in the manifold 686 and is in communication with the large lumen 691 and is provided with a fitting 747 which is adapted to receive the perfusion balloon catheter 693 and the therapeutic balloon catheter 692 as hereinafter described. Another tube 751 is provided in the manifold 686 and is in communication with the aspiration lumen 701. It is provided with the fitting 752. Another tube fitting 753 is mounted in the manifold 686 and is in communication with the irrigation lumen 702 and is provided with a fitting 754.

The various fittings for the catheter as hereinbefore described are adapted to be connected into a control console 771. The control console 771 consists of a rectangular case 772 which is provided with a front panel 773.

A plurality of balloon inflation deflation devices 776 of a conventional type typically called endoflaters are mounted within the case 772 and have control handles 777 extending through vertically disposed slots 778 provided in the front panel. These endoflaters 776 are labeled as shown in FIG. 24 and are connected by tubing (not shown) through pressure gauges 781 mounted in the front panel 773 and are provided with needle indicators 782 to indicate the pressure being applied by the endoflater to the tubing. The tubing is connected in such a manner so that the endoflater 776 and the associated pressure gauge 781 are connected to a tube 786 which is provided with a mating fitting 787 adapted to mate with a fitting 688 so that it is in communication with the inflation lumen 682 of the proximal occlusion balloon catheter 676. In a similar manner, the tubing 788 is provided with a fitting 789 which mates with a fitting 718 of the balloon inflation lumen 711 of the perfusion balloon catheter 693 for inflating balloon 709. Similarly, tube 791 with its mating fitting 792 is adapted to mate with the fitting 737 for inflating the balloon 734. Similarly, the tube 793 with its fitting 794 mates with the fitting 727 in communication with the balloon inflation lumen 726 for inflating the balloon 724 of the therapeutic catheter 692. Another tube 796 which is provided with its fitting 797 mates with the fitting 752 that is in communication with the aspiration lumen 701. The tube 796 is in communication with the inlet of a blood pump 801 of a suitable type as for example a roller pump well known to those skilled in the art which is mounted within the case 772 and which is connected to a source of electrical power through electrical plug 802 connected into the case 772. The roller pump 801 is provided with an on/off switch 803 mounted on the front panel 773. After it passes through the pump 801, blood is supplied to a blood filter 806 of a conventional type and then is supplied through a tube 811 having a fitting 812 adapted to mate with the fitting 716 of the perfusion balloon catheter which is in communication with the perfusion lumen 712.

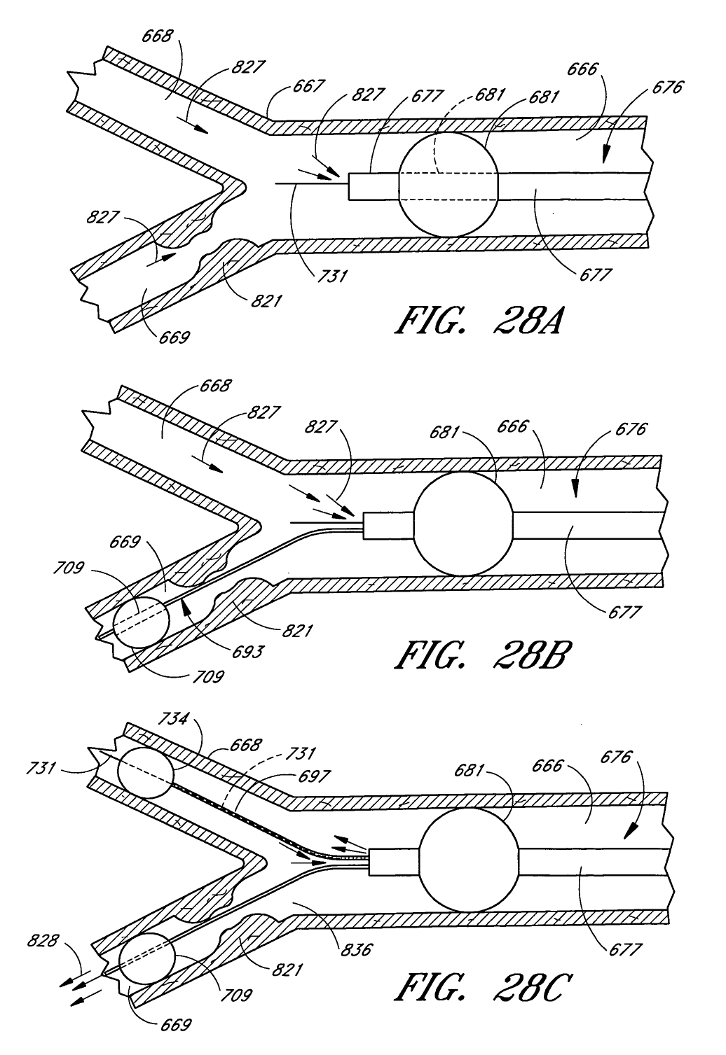

A three-way valve 816 is associated with each of the endoflaters 776 and has a control knob 817 extending through the front panel 753 and is adaptable to be moved between three positions with a center off position and an aspiration position in a counter-clockwise direction and a pressurized position in a clockwise position as viewed in FIG. 27.

Operation and use of the apparatus 651 may now be briefly described as follows. Let it be assumed that it is desired to treat a stenosis occurring in a bifurcation in a carotid artery as depicted by the illustrations shown in FIGS. 28A through 28D. As shown in the illustration in FIG. 28A, let it be assumed that a stenosis is present adjacent the bifurcation 667 and in the external carotid 668 and that it is desired to treat this stenosis in accordance with the apparatus 651 of the present invention in performing the method of the present invention. The proximal occlusion balloon catheter 676 is loaded with the therapeutic balloon catheter 692 slidably mounted over the perfusion balloon catheter 693 and both are slidably mounted in the main lumen 691. The balloon-on-a-wire catheter 697 is slidably mounted in the lumen. While the patient is being prepared for the procedure, all of the lumens in the catheters of the apparatus are flushed with saline to remove all air from the lumens. They are then connected to the control console 771 in the manner hereinbefore described and as shown in FIG. 24. An incision 826 (see FIG. 24A) is made in the femoral artery in the left leg of the patient and a guiding catheter (not shown) similar to the type utilized in angioplasty is introduced through the femoral artery 659. This guiding catheter is advanced until it is near the aorta arch 661. Thereafter, the first or proximal occlusion balloon catheter 676 has its distal extremity 679 introduced into the guiding catheter and advanced in the guiding catheter. It is advanced so that its distal extremity 679 enters the common carotid and is near the bifurcation 667. The balloon 681 is inflated by operating the control handle 777 associated with the proximal occlusion balloon 681 as shown in FIG. 28A to create the desired pressure within and to inflate the elastic balloon 681 so that it occludes the common carotid just proximal of the stenosis 824. As soon as this occurs, the roller pump 801 is turned on by operating the on/off switch 803 to create a negative pressure on the distal side of the balloon 681 to cause blood to flow in a reverse direction as shown by arrows 827 to thereby change the directional flow of blood from the internal and external carotids away from the brain rather than to the brain. The blood travels into the aspiration lumen 701 as indicated by the arrows 827 and into the tube 751 through fittings 752 and 797 and tube 796 to the roller pump 803. The blood after passing through the roller pump 803 passes through a blood filter 806 and then passes into the tube 811 and the fitting 812 and connected to the fitting 789 of the perfusion catheter 693. Alternatively, the fitting 812 can be which is connected to another fitting 831 mounted on a tube 832 introduced into the venous side of the circulatory system of the patient's body, as for example into the vein in the right leg of the patient 652 as shown in FIG. 24. Any debris or emboli in the aspirated blood being pumped will be filtered out by the blood filter 806.

As soon as or during the time this retrograde circulation of blood is established through the roller pump 801, the perfusion balloon catheter 693 extending proximally from the fitting 747 is advanced into the internal carotid 669 past the stenosis 821 at the bifurcation 667. If necessary, a guide wire can be utilized which can be introduced through the perfusion lumen 712 to aide in advancing the perfusion balloon catheter 693 into the internal carotid 669. Any emboli or debris dislodged from the stenosis 621 by crossing the same either by the guide wire or by the distal extremity of the catheter 693 will be picked up by the retrograde flow of blood which is being aspirated through the proximal occlusion balloon catheter 676 to thereby prevent any emboli or debris from entering the brain of the patient. The elastic perfusion balloon 709 is then inflated as shown in FIG. 28B using the appropriate endoflater to inflate the balloon to the desired pressure while watching the associated pressure gauge. As soon as occlusion occurs, perfusion of blood can be started as hereinafter described.

Prior to or after the balloon 709 of perfusion catheter 693 has been inflated, the balloon-on-a-wire catheter 697 extending proximally of the fitting 741 is advanced into the external carotid 669 as shown in FIG. 28C. The balloon 734 is then expanded by use of the appropriate endoflater to supply an inflating medium through the fitting 737 to occlude the external carotid 669. As soon as occlusion has been accomplished in both the external and internal carotids, retrograde flow of blood is terminated by shutting off the roller pump 801. It should be appreciated that if desired, automatic controls can be provided whereby when a certain pressure is reached in each of the balloons 709 and 734 the roller pump would automatically be shut off to stop retrograde flow. By this procedure, it can be seen that the lesion of stenosis 821 has been bracketed by the balloons 681, 709 and 734. Prior to that occurring, retrograde flow of blood is established to prevent any emboli or debris from moving towards the brain.

As soon as retrograde flow of blood has been terminated, perfusion of blood is started. This can be accomplished by connecting a cannula (not shown) to the fitting 716 of the perfusion catheter 706 and to obtain a supply of blood from the femoral artery in the other leg of the patient. Alternatively, an outside blood supply can be used. Thus fresh blood will be supplied from the femoral artery of the patient directly into the perfusion balloon so that it is discharged distally of the perfusion balloon 709 as shown by the arrows 828 to continue to supply blood to the carotid artery. It has been found that it is unnecessary to a supply perfusion of blood to the external carotid artery because there is sufficient auxiliary circulation in that carotid artery during the time the procedure is taking place.

In the event there is inadequate pressure on the arterial blood being perfused to overcome the resistance in the lumen 669, the roller pump 801 can be utilized by merely operating the same in a reverse direction and connecting it between the cannula and the perfusion catheter.

After the lesion or stenosis 821 has been bracketed as hereinbefore described and a working space 836 formed adjacent the stenosis or lesion 821, a therapeutic procedure can be employed. By way of example this can consist of advancing the therapeutic balloon catheter 692 over and axially of the perfusion catheter 693 to bring its balloon 724 into registration with the stenosis 821 as shown in FIG. 28D. Thereafter, the balloon 724 can be inflated by use of the appropriate endoflater as hereinbefore described to cause the inelastic balloon to be pressurized to a pressure of 10 to 15 atmospheres to compress the stenosis 821. Prior to or during this procedure it may be desirable to introduce a saline or heparin solution or a radiopaque contrast liquid into the working space 836. This can be accomplished by introducing this liquid through the injection lumen 702. If desired, this can be accomplished prior to terminating the aspiration procedure hereinbefore described. Also it should be appreciated that if desired a small endoscope can be inserted through one of the lumens to view the area within the working space. Alternatively, if desired an ultrasonic probe can be utilized to view the area in which the lesion is disposed.

As hereinbefore described with a previous embodiment, in place of the therapeutic balloon catheter, other types of catheters can be utilized as for example one incorporating an atherectomy device of the type hereinbefore described to facilitate removal of the stenosis. It is readily apparent that during these procedures if it is necessary to supply a saline solution or a heparinized solution into the working space that the working space can also be continued to be aspirated to remove any debris or emboli which occur during the procedure.

Let it be assumed that the desired therapeutic actions have been undertaken and that the stenosis 821 has been reduced and substantially eliminated so that there is adequate flow through the internal carotid. If it can be seen that there also is a stenosis in the external carotid, the balloon-on-a-wire catheter 697 and the perfusion catheter 693 can be withdrawn and moved so that they enter the opposite carotid to permit therapeutic treatment of a stenosis occurring in the other carotid.

When all the desired therapeutic procedures have been accomplished, the supply of saline or contrast solution can be terminated and the therapeutic balloon 724 deflated. The balloon 734 of the balloon-on-a-wire catheter can be deflated as well as the perfusion balloon 709. Perfusion of blood through the perfusion catheter can be terminated. The perfusion balloon catheter 693 and the balloon-on-a-wire catheter 697 can be retracted into the main multi-lumen tubular member 677 of the proximal occlusion balloon catheter after which the perfusion balloon catheter can be withdrawn carrying with it the other catheters disposed therein. Thereafter, the guiding catheter can be removed and a suture applied to the incision made to gain access to the femoral artery.

It is readily apparent that similar procedures can be carried out with respect to other vessels in the body, such as saphenous vein grafts in the heart, and particularly with respect to vessels in the brain where it is difficult if not impossible to employ surgical procedures as for example with respect to the basilar arteries in which bifurcations appear.

As also herein before explained, the catheter apparatus of the present invention can be utilized for deploying stents. Where that is desirable the apparatus of the present invention, perfusion can be accomplished during employment of the stent.

From the foregoing it can be seen that an apparatus and method has been provided for treating occluded vessels and particularly for treating carotid arteries. The apparatus and method of the present invention is particularly advantageous for the carotid arteries because it permits access to portions of the carotid arteries which are not accessible by surgery.

The catheter apparatus assembly and method of the present invention are also particularly useful for treating other occluded vessels but particularly the carotid arteries because it makes possible the removal of plaque without endangering the patient. An operating or working space is provided while shunting blood around the working space so that there is continued blood flow in the vessel to support the functions which are normally supported by the vessel. As also pointed out above, the apparatus and method of the present invention are particularly useful in connection with vessels having bifurcations therein and in which the stenosis occurs at or near the bifurcation. From the foregoing it can be seen with the apparatus and method of the present invention, retrograde flow of blood is accomplished during deployment of the device to prevent undesired travel of emboli. Occlusion of the vessels is provided to obtain a working space by bracketing the working space with balloons while at the same time maintaining perfusion of blood making it possible to utilize a substantial period of time for undertaking therapeutic procedures with respect to the bracketed stenosis.

In connection with the present apparatus and method for treating occluded vessels, it has been found that it is possible to utilize the apparatus and method without perfusion and other procedures involving the carotid arteries and saphenous vein grafts for periods of time extending over five minutes and greater which has made it possible to simplify the apparatus and the method utilized in conjunction therewith.

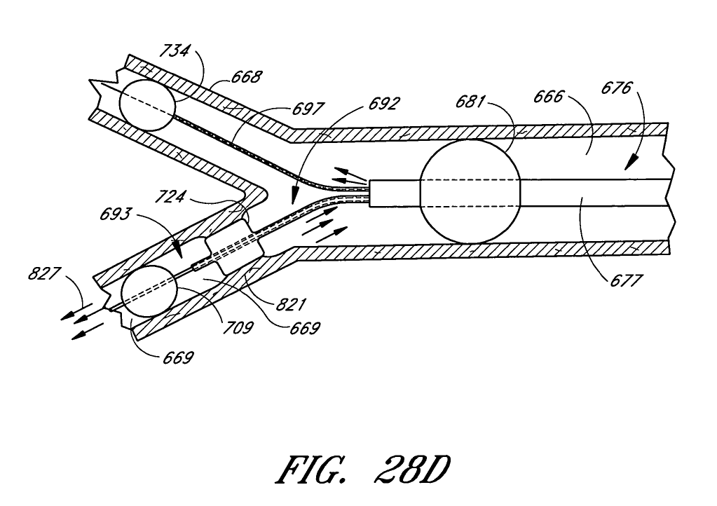

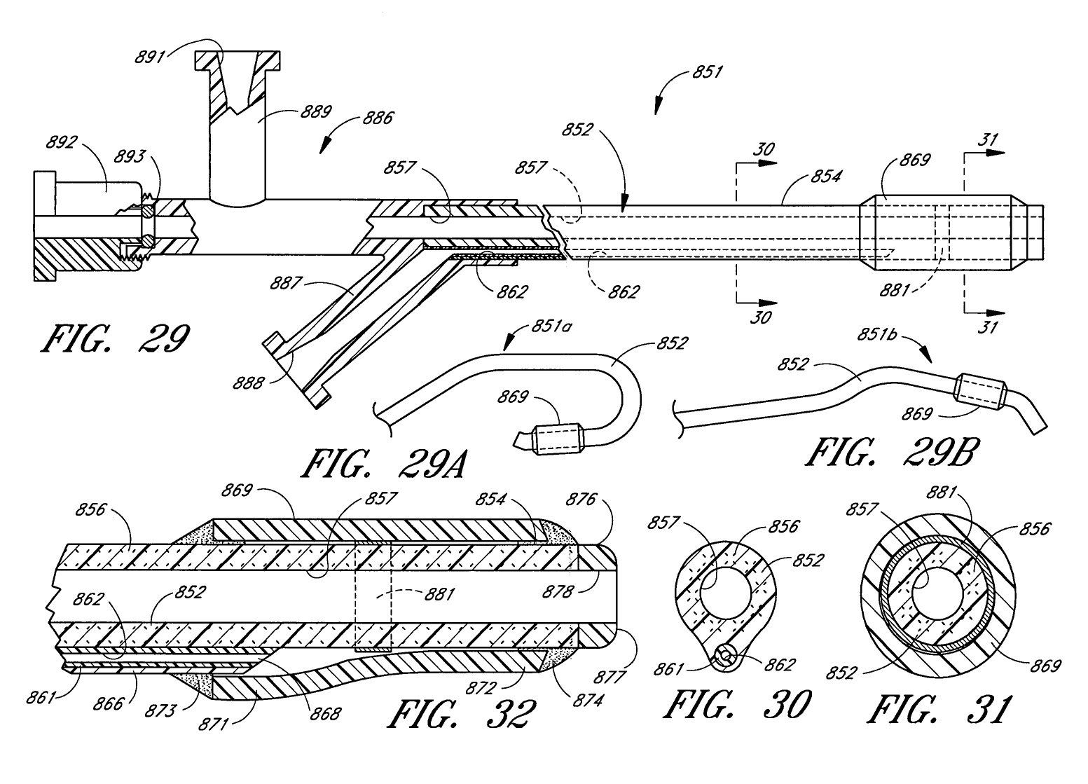

With respect to an apparatus or assembly which does not require the use of perfusion, a main catheter 851 utilized as a part of the apparatus is shown in FIGS. 29, 30, 31 and 32 consists of a flexible elongate tubular member 852 formed of a suitable material such as plastic of the type hereinbefore described and which has proximal and distal extremities 853 and 854. The tubular flexible elongate tubular member 852 can be of various sizes as for example for a saphenous vein graft catheter it can be 8 to 9.5 French in balloon profile with a length ranging from 80 cm to 120 cm. The flexible elongate tubular member 852 can be formed of a suitable material such as PEBAX, Nylon, Hytrel, polyurethane or polyethylene. A flexible braid 856 (see FIGS. 30, 31 and 32) formed of a suitable material such as stainless steel is embedded within the wall of the flexible elongate tubular member 852 as shown and extends from the proximal extremity 853 to the distal extremity 854. The braid 856 can be formed of a suitable stainless steel such as a wire or ribbon having a thickness of 0.001″. The braid 856 provides additional torquability and also inhibits the kinking of the flexible elongate tubular member 852 when it must extend over a tight radius. The flexible elongate tubular member 852 is provided with a large central lumen 857 having a suitable diameter such as 0.065 or greater extending from the proximal extremity to the distal extremity.

If it is desired to provide a flexible elongate member 852 which has a greater flexibility at the distal extremity, a different material can be used in the distal extremity 854. For example, the distalmost 5-15 centimeters can be formed of a material such as PEBAX having a Shore D hardness of 35-50 with the remainder of the flexible elongate member 852 having a Shore D hardness of 65-75.

A supplemental flexible elongate tubular member 861 is provided which has incorporated therein a balloon inflation lumen 862. The supplemental flexible elongate tubular member 861 can be of a suitable size as for example an I.D. of 0.014″ and an O.D. of 0.018″ and formed of a suitable material such as a polyimide. The supplemental flexible elongate tubular member has a length which is almost as long as the flexible elongate tubular member 852 and overlies the outside wall of the flexible elongate tubular member 852 and extends from the proximal extremity to near the distal extremity as shown in FIGS. 29 and 32. A tube 863 of a suitable material such as Pebax extends over the length of the polyimide tubing 861 and is secured to the flexible elongate tubular member 852 by a shrink tube 866 extending from the proximal extremity 853 to the distal extremity 854, after which the shrink tube 863 is subjected to heat. The shrink tube 866 is then subjected to a hot melt process of a temperature around 350° F. for a period of time until the Pebax tube 863 melts, after which the shrink tubing 866 can be stripped off so that there remains a relatively uniform mass formed of Pebax that surrounds the braid 857 and the polyimide tube 861 which forms the supplemental flexible elongate tubular member 861. The polyimide tube which forms the supplemental flexible elongate tubular member 861 thus provides an inflation lumen 867 extending from the proximal extremity and to the distal extremity and opens through an opening 868 into the interior of an occlusion balloon 869 which is bonded to and coaxially mounted on the distal extremity of the flexible elongate member 852 in the manner shown in FIG. 32. The polyimide tubing is provided to give the balloon inflation lumen shaft 561 greater strength than that which is provided by the Pebax itself.

As can be seen from FIG. 32, the supplemental flexible elongate tubular member 861 is terminated short of the distalmost extremity of the flexible elongate tubular member 852 by approximately 1 cm. The occlusion balloon 869 is formed of various compliant or non-compliant materials. Suitable compliant materials include elastomers such as C-Flex latex, silicones and polyurethanes. Suitable non-compliant materials would be polyethylene, PET and Nylon. A composite material can also be used such as a combination of PET and an elastomer. The occlusion balloon 869 should have a strength so that it can readily accommodate any pressure of one atmosphere and as high as four atmospheres, or approximately 60 psi. The occlusion balloon 869 is cylindrical and is provided with proximal and distal extremities 871 and 872 which are secured by a suitable medical grade adhesive. Alternatively, fuse bonding may be used. Thus a seal 873 formed of this adhesive bonds the proximal extremity 871 of the occlusion balloon 869 over the outer surface of the distal extremity of the flexible elongate tubular member 852 and the supplemental flexible elongate tubular member 861. Similarly, a seal 874 bonds the distal extremity 872 to the distal extremity of the flexible elongate tubular member 852 to provide an air-tight space within the balloon accessible through the opening 868. A soft cylindrical tip 876 formed of suitable material such as Pebax is bonded to the distal extremity of the flexible elongate tubular member 862 and is provided with a rounded surface 877 which extends forwardly and has a passage 878 therein in communication with the lumen 857 and the flexible elongate tubular member 852. A cylindrical radiopaque marker 881 formed of a suitable material such as platinum, platinum-iridium or gold is mounted on the distal extremity of the flexible elongate tubular member 852 in a position so it is substantially equidistant of the ends of the occlusion balloon 869.

A main adapter or fitting 886 formed of a suitable material such as plastic is mounted on the proximal extremity 853 of the flexible elongate tubular member 852. It is provided with a first Luer fitting 887 which provides a balloon inflation port 888 in communication with the balloon inflation lumen 862. It is also provided with another Luer fitting 889 which is provided with an aspiration port 891 in communication with the main central lumen 857. The main adapter 886 is also provided with a Tuohy-Borst fitting 892 which is in communication with the central lumen 857. The Tuohy-Borst fitting 892 is adapted to receive therapeutic devices, as for example a balloon-on-a-wire device as hereinafter described and is adapted to form a liquid-tight seal therewith by an o-ring 893.

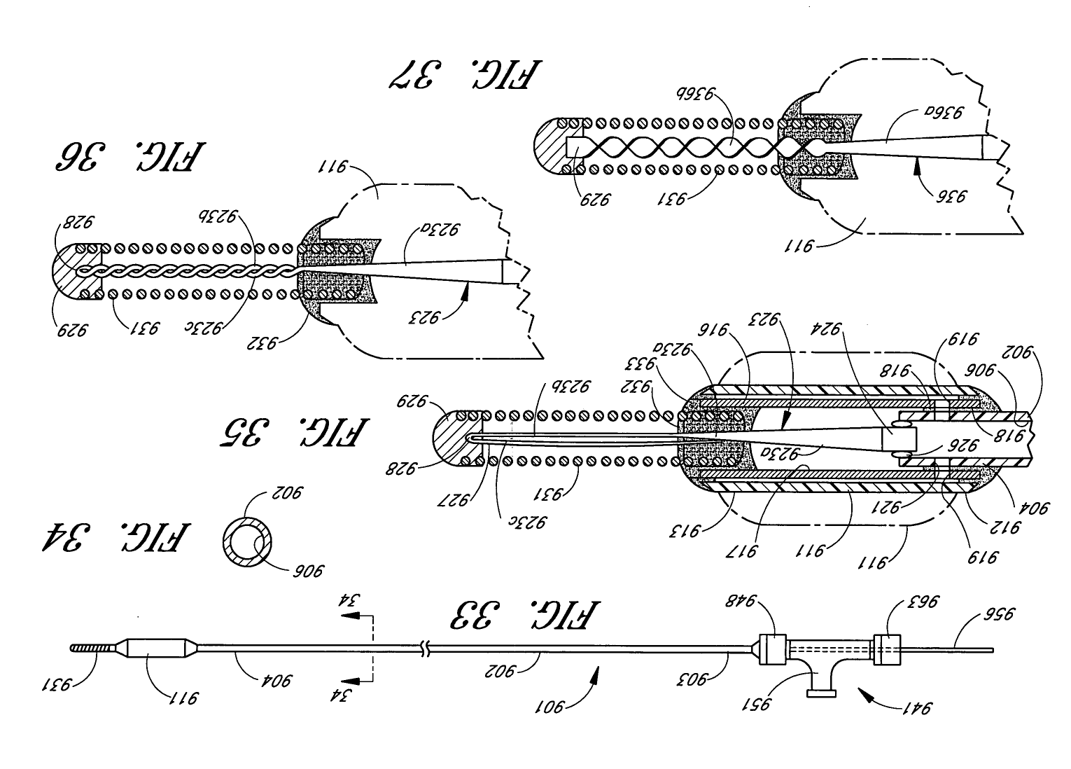

A balloon-on-a-wire device 901 incorporating the present invention is shown in FIGS. 33 and 34. The device 901 consists of a guide wire 902 formed of a suitable material such as stainless steel and having a suitable diameter as for example ranging from 0.010″ to 0.032″ but preferably a diameter ranging from 0.014″ to 0.018″. It is preferable that the guide wire 902 be formed of a nickel titanium alloy typically called Nitinol which has the advantage that it is more flexible and has greater kink resistance characteristics than another suitable material such as stainless steel.

It has a suitable length as for example 150 cm. The guide wire 902 is provided with proximal and distal extremities 903 and 904 and is provided with a central lumen 906 extending from the proximal extremity to the distal extremity. The lumen can be of a suitable size as for example 0.010″ I.D. for an 0.014″ O.D. guide wire.

An occlusion balloon 911 is coaxially mounted on the distal extremity 904 of the guide wire 902. The occlusion balloon 911 is preferably formed of the same material as the occlusion balloon 869 on the main catheter 851. The occlusion balloon 911 has proximal and distal extremities 912 and 913. A tube 916 formed of a suitable material such as a polyimide is disposed within the occlusion balloon 911 and has a bore 917 extending therethrough which is sized so that it is slightly larger than the outside diameter of the guide wire 902 so that its proximal extremity can be slipped over the distal extremity 904 of the guide wire 902 and then bonded thereto by suitable means such as an adhesive 918. A plurality of circumferentially spaced apart radially extending inflation holes 919 are provided in the proximal extremity of the tube 916 and are in alignment with similarly spaced holes 921 provided in the distal extremity 904 of the guide wire 902 so that they are in communication with the central lumen 906 of the guide wire 902. The inflation holes 919 as shown are in communication with the interior of the occlusion balloon 911 so that fluid passing from the passage 906 can be utilized for inflating the occlusion balloon 911.

A solid core wire 923 formed of a suitable material such as stainless steel is provided with a proximal tapered extremity 924. The core wire 923 is sized so it is adapted to fit within the lumen 906 of the guide wire 902 and is secured therein by suitable means such as an adhesive 926 or alternatively a weld. The core wire 923 has a tapered portion 923a which commences at the proximal extremity 924 and which is tapered so that the cross-sectional diameter progressively decreases to the distal extremity of the occlusion balloon 911. The core wire 923 is also provided with additional portions 923b and 923c which can be of substantially constant diameter as for example 0.003″. The portion 923 is folded over with respect to the portion 923b so that the portions 923b and 923c lie in a plane to facilitate shaping of the distal extremity of the guide wire 902 during use of the same. The core wire 923 is provided with a distal extremity 927 in which a bend 928 is formed between the two portions 923b and 923c. The bend 928 is secured within a hemispherical solder bump or protrusion 929 which is carried by the distal extremity of a coil 931 formed of a suitable radiopaque material such as platinum or a platinum alloy. The platinum coil 931 can have a suitable outside diameter as for example 0.014″ corresponding to the diameter of the guide wire 902 and can have a suitable length ranging from 1 to 3 cm. The proximal extremity of the coil 931 is secured to the distal extremity of the polyimide tube 916 by suitable means such as an adhesive 932 which can be the same adhesive or a different adhesive 933 utilized for securing the distal extremity 913 of the balloon to the polyimide tube 916 to form a fluid-tight seal between the distal extremity of the occlusion balloon 911 and the distal extremity of the polyimide 916. From this construction it can be seen that the portions 923b and 923c of the core wire 923 in addition to serving as a shaping ribbon are also utilized as a safety ribbon to ensure that the tip 928 and the spring 931 cannot be separated from the guide wire 902. The proximal extremity 912 of the balloon 911 is also secured to the proximal extremity of the polyimide tube 916 and also to the distal extremity 904 of the guide wire 902 to form a fluid-tight seal with respect to the occlusion balloon 911 so that the occlusion balloon 911 can be inflated and deflated through the inflation holes 919 and 921.

Alternative constructions for the distal extremity of the core wire 923 are shown in FIGS. 36 and 37. In FIG. 26 it can be seen that the portions 923b and 923c have been twisted to in effect provide a twisted pair serving as a safety ribbon and as a shaping ribbon. In the embodiment shown in FIG. 37, the core wire 936 is provided with a tapered portion 936a which is the same as the tapered portions of 923a hereinbefore described. However, the core wire 936 has been provided with a distal portion 936b which has been flattened to a suitable thickness as for example a width of 0.006″ and a thickness of 0.003″ and then twisted to form a helix as shown in which the distal extremity is embedded within the solder 929. Such a helix 936 can serve as a safety ribbon and also can be shaped to some extent.

A removable inflation fitting 941 or valve attachment 941 is mounted on the proximal extremity of the guide wire 902 and forms a part of the balloon-on-a-wire device 901. The fitting or attachment 941 is formed of a suitable material such as a polycarbonate and is provided with a central bore 942. The attachment or fitting is slid externally over the proximal extremity 903 of the guide wire 902. Means is provided for forming a fluid- tight seal between the proximal extremity 903 of the guide wire 902 and a body 943 of the fitting 941 and consists of an o-ring 946 (see FIG. 38) seated in a well 947. A thumb screw 948 is threadedly mounted on the body 943 and is provided with an inwardly extending circular protrusion 949 that is adapted to engage the o-ring 946 and to compress the same to form a fluid-tight seal when the protrusion 949 is moved inwardly toward the o-ring 946 as the thumb screw 948 is rotated in a clockwise direction. The o-ring 946 decompresses or springs back when released upon rotation of the thumb screw 948 in a counterclockwise direction so that the fitting 941 can be removed from the proximal extremity 903 of the guide wire 902. The body 943 also includes a Luer fitting 951 which provides an inflation port 952 that is in communication with the bore 942 in the body 943 and which is also in communication with the open proximal extremity of the guide wire 902 and the lumen 906 therein.

Means is provided for plugging the bore 906 when the removable attachment or fitting 941 is removed and consists of a plug mandrel 956 formed of a suitable material such as 0.014″ stainless steel solid rod. It is necessary that this rod have a diameter which is greater than the diameter of the lumen 906 and the guide wire 902. The plug mandrel 956 is provided with a progressive portion 956a that tapers down from as, for example from 0.014″ to a suitable diameter as for example 0.008″ to a cylindrical portion 956b.

Means is provided for forming a fluid-tight seal between the plug mandrel 956 which forms a plug mandrel and the body 943 of the attachment or fitting 941 and consists of an o-ring 961 providing suitable sealing means seated within a well 962 provided in the body 943. A thumb screw 963 threadedly engages the body 943 and is provided with a cylindrical protrusion 964 which engages the o-ring and compresses it to form a fluid-tight seal with respect to the plug mandrel 956 by rotation in a clockwise direction of the thumb screw 963. The plug mandrel 956 can be released by a counterclockwise rotation of the thumb screw 963 permitting decompression of the o-ring 961.

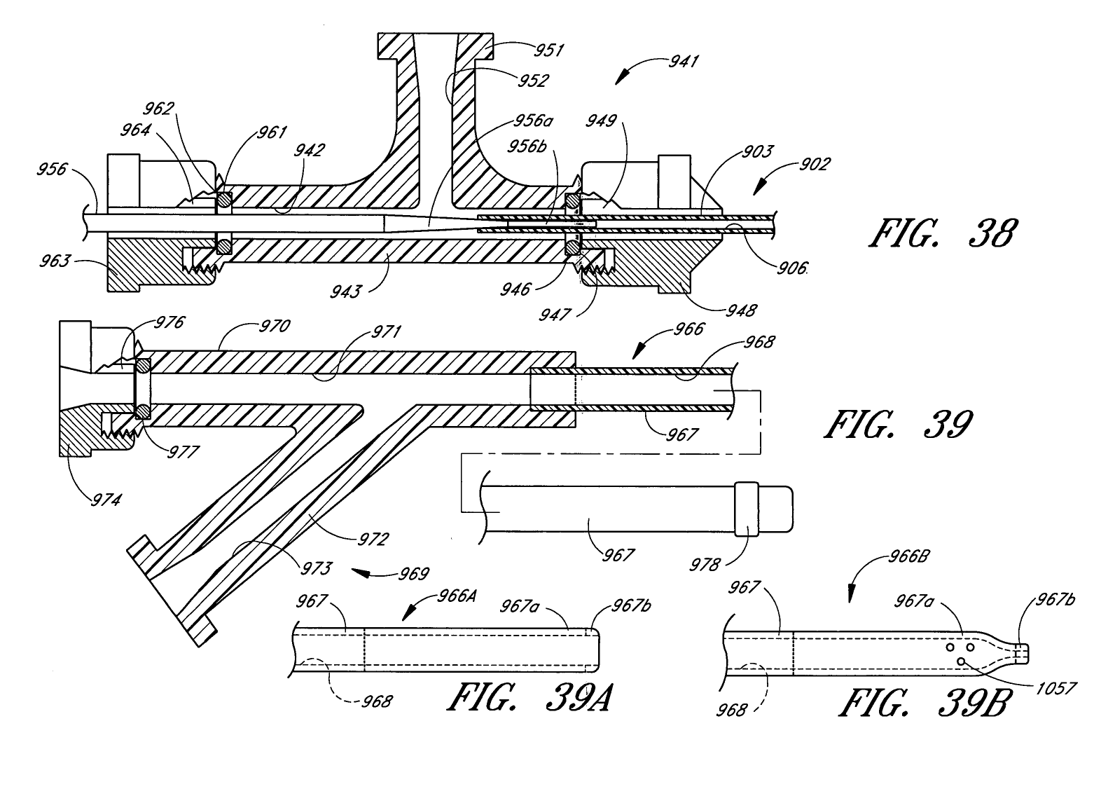

An irrigation catheter 966 incorporating the present invention is shown in FIG. 39 and consists of a flexible elongate tube 967 formed of a suitable material such as polyethylene, PEBAX, Hytrel or Teflon having a suitable size as for example an outside diameter of 0.066″ and an inside diameter of 0.058″ and having a length of approximately 150 cm. A lumen 968 is provided therein and extends from the proximal extremity to the distal extremity and is in communication with an adapter 969 provided on the proximal extremity of the tube 967. The adapter 969 is provided with a body 970 formed of a suitable material such as plastic and is provided with a bore 971 extending therethrough. The adapter 969 is provided with a side arm 972 which carries a conventional Luer-type connection and provides an irrigation port 973 in communication with the bore 971. A thumb screw 974 threadedly mounted on the body 970 carries a cylindrical protrusion 976 adapted to compress an o-ring 977 carried by the body 970 into engagement with a therapeutic catheter of the type hereinafter described. A radiopaque tip marker 978 of a suitable type, as for example one formed as a platinum-iridium band 978 is provided on the distal extremity of the flexible elongate element 967 to facilitate positioning of the irrigation catheter as hereinafter described.

Operation of the apparatus shown in FIGS. 29 through 39 in performing the method of the present invention for treating occluded vessels may now be briefly described as follows utilizing the cartoons which are shown in FIGS. 40-46. Let it be assumed that it is desired to treat a vessel 981 in the human body as for example a saphenous vein graft having at least a partial occlusion or stenosis 982 which is formed by plaque in the vessel. The main catheter 851 is introduced into the body through a conventional procedure such as for example by making an incision into the femoral artery in a leg of the patient.

Thereafter the main catheter 851 can be introduced into the femoral artery by use of a large conventional guiding catheter because the main catheter 851 is of a relatively large size, as for example 8 to 9.5 French. In order to eliminate the need for such a large guiding catheter, a smaller conventional guiding catheter 986 of the type shown in FIG. 40 can be utilized which can be introduced through the main catheter 851. Utilizing such a catheter, the main catheter 851 can be inserted independently through a conventional sheath (not shown) in the femoral artery and thereafter the guiding catheter 986 is introduced through the main catheter 851 so that its distal extremity 989 is in the vessel. Alternatively, the guiding catheter 986 can be deployed into the main catheter 851 and the guiding catheter 986 introduced at the same time into the femoral artery.

The guiding catheter 986 is conventional and thus will not be described in detail. It consists of a flexible elongate tubular member 987 (see FIG. 40) formed of a suitable material such as plastic having proximal and distal extremities 988 and 989. The distal extremity 989 is provided with a preformed bend as shown. An adapter 992 is mounted on the proximal extremity 988 and consists of a body 993 in the form a wye in which the central leg 994 is provided with a flow passage (not shown) therein in communication with the central lumen (not shown) extending from the proximal extremity 988 to the distal extremity 989 of the flexible elongate tubular member 987. The body 993 is provided with a side leg 996 which also is in communication with a lumen (not shown) extending from the proximal extremity 988 to the distal extremity 989. A knob 997 carrying an o-ring (not shown) secures the adapter 992 to the proximal extremity 988 with a fluid-tight seal. Another knob 998 is provided which is carried by the central leg 994 of the body 993 and is provided with an o-ring (not shown) which can be moved to close the flow passage in the central leg 994, or alternatively it can be opened to receive a guide wire which can be utilized for advancing the guide catheter 986 if that be necessary and then forming a fluid-tight seal with respect to the guide wire.

Assuming that the guiding catheter 986 has been inserted into the main catheter 851 before insertion of the main catheter 851 into the femoral artery, both catheters can be inserted in unison while advancing the distal extremity of the guide catheter 986 so that it precedes the distal extremity of the main catheter 851 and serves to guide the main catheter 851 into the vessel of interest, as for example the vessel 981 having the stenosis 982 therein. The main catheter 851 is then advanced so that its distal extremity is at the proximal side of the stenosis 982. By way of example, the main catheter 851 can be advanced through the aortic arch of the heart and thence into a saphenous vein graft so that the occlusion balloon 869 on its distal extremity is positioned proximal of the stenosis 982. As soon as this has been accomplished, the guiding catheter 986 can be removed.

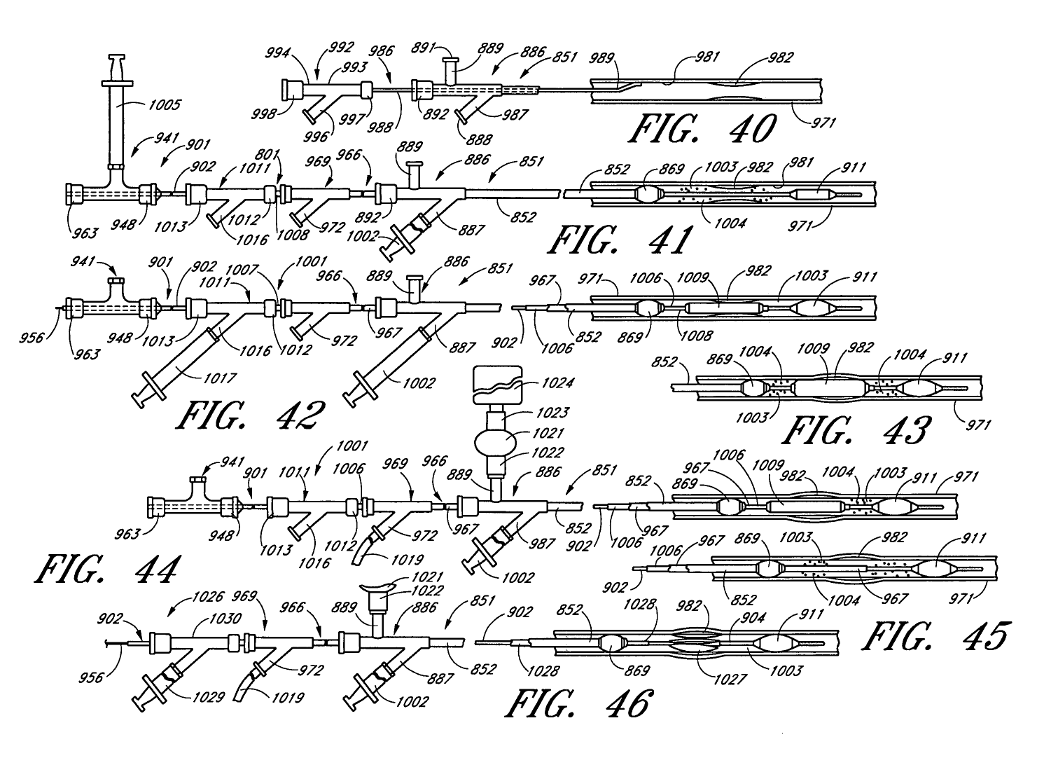

As soon as the distal extremity of the main catheter 851 has been deployed so that it is just proximal of the stenosis 982 to be treated, an assembly shown in FIG. 41 is introduced into the main catheter 851. This assembly can be provided by preloading the irrigation catheter 966 onto the therapeutic catheter 1001 by inserting the distal tip of the therapeutic catheter 1001 through the fitting 969 of the irrigation catheter 966 and advancing the therapeutic catheter 1001 until its therapeutic balloon 1009 exits from the irrigation catheter 966. The balloon-on-a-wire catheter 901 also is preloaded by removing the valve attachment 941 and then inserting the proximal end 903 into the guide wire lumen at the distal tip of the therapeutic catheter 1001 and then advanced proximally until the proximal end protrudes out of the proximal end of the therapeutic catheter. The valve attachment 941 is then reattached to the proximal end 903. The preassembled irrigation catheter 966, the therapeutic catheter 1001 and the balloon-on-a-wire catheter 901 are then introduced in unison as an assembly into the main catheter 851. The balloon-on-a-wire device 901 is then advanced until the distal extremity is near the distal extremity of the main catheter 851 but before the distal extremity has been advanced through the stenosis 982.

Let it be assumed that it is now desired to inflate the occlusion balloon 869 carried by the main catheter 851. This can be accomplished in a suitable manner such as with an inflation-deflation device represented schematically by a syringe 1002 secured to the fitting 887 (see FIG. 41) and supplying a balloon inflation fluid through the balloon inflation lumen 862 to inflate the occlusion balloon 869 to an occlusion pressure ranging from 1 to 3.9 atmospheres and preferably approximately one to two atmospheres to engage the side wall forming the vessel 981 to occlude the vessel 981 and to prevent further blood flow through the vessel and to thereby provide a working space 1003 distal of the occlusion balloon 869. As soon as the occlusion balloon 869 has been inflated, the balloon-on-a-wire device 901 can be advanced across the lesion or stenosis 982 until the deflated occlusion balloon 911 carried thereby is distal of the stenosis 982. It is safe to cross the stenosis 982 because the flow of blood through the stenosis 982 has been occluded by the occlusion balloon 869. Thus if any of the plaque forming the stenosis is dislodged by the occlusion balloon 911 on the balloon-on-a-wire device 901 as the occlusion balloon 911 is crossing the stenosis 982, the plaque particles or emboli 1004 will not be carried off by blood. The positive pressure of blood in secondary collaterals or vasculature will prevent emboli from traveling downstream into the secondary vasculature. If desired, aspiration can be supplied to the working space 1003 encompassing the stenosis 982 by placing a suitable vacuum connected to the fitting 889 of the main catheter.

The occlusion balloon 911 can then be readily inflated by use of a syringe 1005 secured to the fitting 951 of the removable valve fitting or attachment 941 of the balloon-on-a-wire device 901 proximal of the fitting 886 and accessible outside the body of the patient. The occlusion balloon 911 is inflated (see FIG. 42) to at least approximately one to two atmospheres to bracket the stenosis and to determine the size of the working space 1003 to provide a chamber. It should be appreciated that the size of this working space or chamber 1003 can be adjusted by changing the position of the occlusion balloon 911 in the vessel 981. If desired, this can be accomplished while the occlusion balloon 911 is inflated.

Now let it be assumed that the occlusion balloon 911 has been inflated with the appropriate working space 1003 and that it is desired to introduce a therapeutic balloon catheter 1001 into the working space 1003 to treat the stenosis 982. If the therapeutic catheter 1001 is not in the main catheter 851 as hereinbefore described, this can be readily accomplished in the present invention by inserting a plug mandrel 956 into the open end of the lumen 906 of the guide wire 902. After the plug mandrel 956 has been inserted, the syringe 1005 can be removed after which the thumb screws 948 and 963 can be loosened to permit the o-rings therein to become decompressed and to release the guide wire 902 and the plug mandrel 956 to permit the fitting or valve attachment 941 to be slipped off to provide a proximal end on the guide wire 902 which is free of obstructions. During removal of the valve attachment or fitting 941, the occlusion balloon 911 remains inflated and continues to be disposed distally of the stenosis 982. The occlusion balloon 869 also remains inflated because the syringe 1002 remains attached to the fitting 886 and is disposed proximal of the stenosis 982.

The conventional therapeutic catheter 1001 then can be delivered over the guide wire 902 if it is not already present. The therapeutic catheter 1001 is provided with a flexible elongate tubular member 1006 having proximal and distal extremities 1007 and 1008 with a central flow passage (not shown) extending between the same. A therapeutic balloon 1009 on its distal extremity is adapted to be inflated to therapeutic pressures ranging from 4-20 atmospheres through a balloon inflation lumen (not shown) carried by the flexible elongate tubular member 1006 through an adapter 1011 mounted on the proximal extremity 1007. The therapeutic balloon 1009 can be considered to be means for performing work carried by the distal extremity 1008 of the flexible elongate tubular member 1006. The adapter 1011 can be removable of the type hereinbefore described or alternatively can be permanently attached thereto. Assuming that it is a removable adapter, the removable adapter 1011 is provided with knobs 1012 and 1013 carrying o-rings (not shown) adapted to establish fluid-tight seals with the flexible elongate member 1006 and the plug mandrel 956, respectively. It is also provided with an inflation port 1016 similar to those hereinbefore described which is in communication with the inflation lumen (not shown) provided in the flexible elongate tubular member 1006 for inflating the therapeutic balloon 1009.

After the balloon catheter 1001 has been positioned by the use of radiopaque markers (not shown) conventionally employed in such devices, the therapeutic balloon 1009 is disposed so that it is in general alignment with the stenosis 982 as shown in FIG. 42. The therapeutic balloon 1009 is then inflated in a conventional manner to perform work by use of an inflation-deflation device schematically represented by the syringe 1017 attached to the inflation port 1016 to the desired pressure to compress the plaque forming the stenosis 982 as shown in FIG. 43 to increase the size of the opening through the stenosis 982 in the vessel 981.

Let it be assumed that during the compression of the plaque forming the stenosis 982, additional emboli 1004 are formed as shown in FIG. 44 by pieces of plaque becoming dislodged from the plaque 982 within the vessel 981. Let it also be assumed that it is desired to remove these emboli before deflation of the occlusion balloons 869 and 911 disposed proximally and distally of the stenosis 982. To accomplish this, the therapeutic balloon 1009 is deflated by use of the syringe 1017. *As soon as this has been accomplished, a saline solution can be introduced through the irrigation catheter 966 by connecting a tube 1019 carrying the saline solution from a suitable source as for example a free or pressurized saline bag (not shown) and delivered through the irrigation port or side arm 972 where it is carried through the large central lumen of the irrigation catheter 966 so that the saline solution is discharged into the working space 1003 disposed between the occlusion balloons 911 and 869 as shown in FIG. 44. At the same time suitable aspiration means is connected to the aspiration port 889 of the adapter 886 and as shown can consist of a hand operated bulb 1021 which has a one way check valve 1022 therein connected to the fitting 889.

The bulb 1021 is provided with another one-way check valve 1023 which is connected to a flexible collection bag 1024. The bulb 1021 makes it possible to generate a vacuum corresponding approximately to 3-30″ of mercury. Thus, by compressing the bulb 1021 by hand, it is possible to create suction within the chamber or space 1003 formed in the vessel between the occlusion balloons 869 and 911 each time the bulb 1021 is compressed and released. Alternatively, the aspiration can be accomplished by use of a syringe in place of the bulb 1021 and the collection bag 1024. Saline liquid supplied through the irrigation catheter 966 carrying the emboli 1018 is aspirated through the central lumen 857 of the main catheter 851. The aspirated liquid in each cycle of operation created by pressing the bulb 1021 is delivered to the collection bag 1024. With such a procedure it has been found that it is possible to aspirate emboli as large as 800 μm. Such removal can be assured by observing when clear liquid exits outside the body from the aspiration port 891. A chamber having a length ranging from 3 cm to 15 cm can be totally cleared of emboli within a short period of time ranging from 5 to 30 seconds. Alternatively, irrigation can be accomplished by removing the therapeutic catheter 1001 after deflating the therapeutic balloon 1009. The irrigation catheter can be advanced over the balloon-on-a-wire device 901 until the distal tip is just proximal of the occlusion balloon 911 as shown in FIG. 45 to provide a greater flow of saline and faster aspiration.

After all of the emboli 1004 have been removed, introduction of saline through the tube 1019 is halted. It should be appreciated that the ports for irrigation and aspiration can be reversed in function if desired. Thereafter, the occlusion balloon 911 is deflated by removing the plug 956 and utilizing a syringe 1005, after which the occlusion balloon 869 is deflated permitting blood flow to be reestablished in the vessel 981. Alternatively, the occlusion balloon 869 can be first deflated and aspiration commenced at that time, permitting emboli trapped distally of the occlusion balloon 869 by blood flowing from the proximal side of the occlusion balloon 869 to be aspirated through the central lumen 857. In order to prevent excessive expansion of the vessel 981 being treated, the pressure of the irrigation liquid is typically maintained under 30 psi. This pressure preferably should be below the occlusion balloon pressure.

If it is desired to deliver a stent to the site of the stenosis formed by the plaque 982, this can be readily accomplished during the same procedure. Typically it is desirable to permit the blood to flow normally for a period of several minutes after which the occlusion balloon 869 can be reinflated by the syringe 1005 and the occlusion balloon 911 can be reinflated by inserting the removable valve attachment 941 if it has been removed of the balloon-on-a-wire device 901 and utilizing the syringe 1003 to reinflate the occlusion balloon 911. The plug mandrel 956 can be inserted to keep the occlusion balloon 911 inflated after which the valve attachment 941 can be removed.

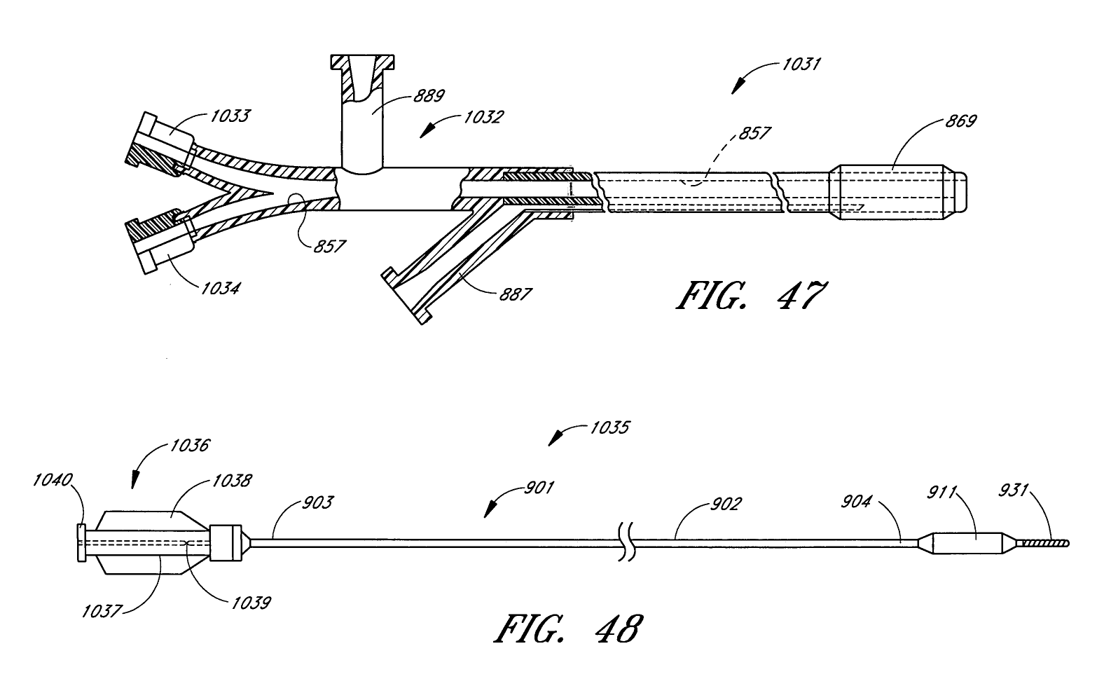

A conventional stent delivery catheter 1026 carrying a stent 1027 on its flexible shaft 1028 is introduced over the balloon-on-a-wire device 901 and delivered to the site of the dilated stenosis 982 (see FIG. 46). The stent 1027 can be of the self-expanding type or of the type which can be expanded by a balloon (not shown) carried by the catheter 1026 by connecting a syringe 1029 to an adapter 1030 of the type hereinbefore described of the stent delivery catheter 1026. After the stent 1027 has been deployed in the dilated stenosis 982, the stent delivery catheter 1026 can be removed after which the occlusion balloon 911 can be deflated followed by deflation of the proximal balloon 861 in the manner hereinbefore described. Also it should be appreciated that if desired in connection with the deployment of the stent delivery catheter 1026 before it is removed but after deflation of its balloon (not shown), it may be desirable to again flush the working space or chamber 1003 between the occlusion balloons 869 and 911 of emboli which may be dislodged during the delivery and deployment of the stent. The irrigation catheter 966 can be deployed in the same manner as hereinbefore described with a saline irrigation solution supplied to the working space 1003 in the manner hereinbefore described and liquid aspirated therefrom by the use of the bulb 1021 in the manner hereinbefore described.

Heretofore the apparatus of the present invention has been utilized for performing a procedure on a saphenous vein graft where there are no branches to be dealt with. An apparatus incorporating the present invention also can be useful in connection with vessels in a human being having branches therein, as for example the carotids. For this purpose, a main catheter 1031 (see FIG. 47) is provided which is very similar to the main catheter 851 with the exception that the adapter 1032 provided on the proximal extremity is provided with catheter ports 1033 and 1034 which are in communication with the large central lumen 857 extending the length of the main catheter. The catheter ports 1033 and 1034 have a construction similar to the exchange catheter and therapeutic catheter port 892 hereinbefore described in connection with the main catheter 851. These two catheter ports 1033 and 1034 are necessary because in a carotid procedure, two balloon-on-a-wire devices are utilized. The main catheter should be larger, as for example as large as 12 French, to provide a larger central lumen to accommodate the two balloon-on-a-wire devices.

One of the balloon-on-a-wire devices can be substantially identical to the balloon-on-a-wire device 901 described. The other balloon-on-a-wire device 1035 as shown in FIG. 48 differs from the device 901 shown in FIG. 33 in that in place of the removable valve attachment 941 there is provided a fixed adapter 1036 which consists of a body 1037 provided with diametrically extending wings 1038 to facilitate grasping of the adapter 1036. The body 1037 is provided with a bore 1039 which is in communication with the lumen 906 in the guide wire 902. The adapter is provided with a Luer-type fitting 1040 to provide a balloon inflation port.

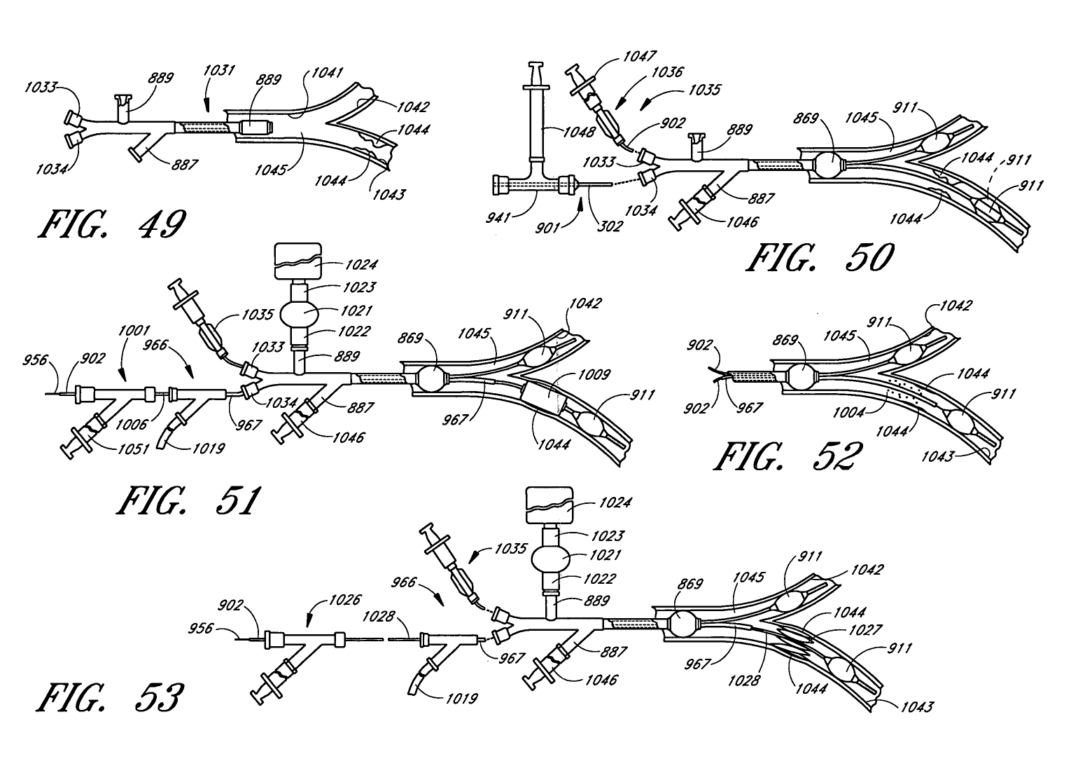

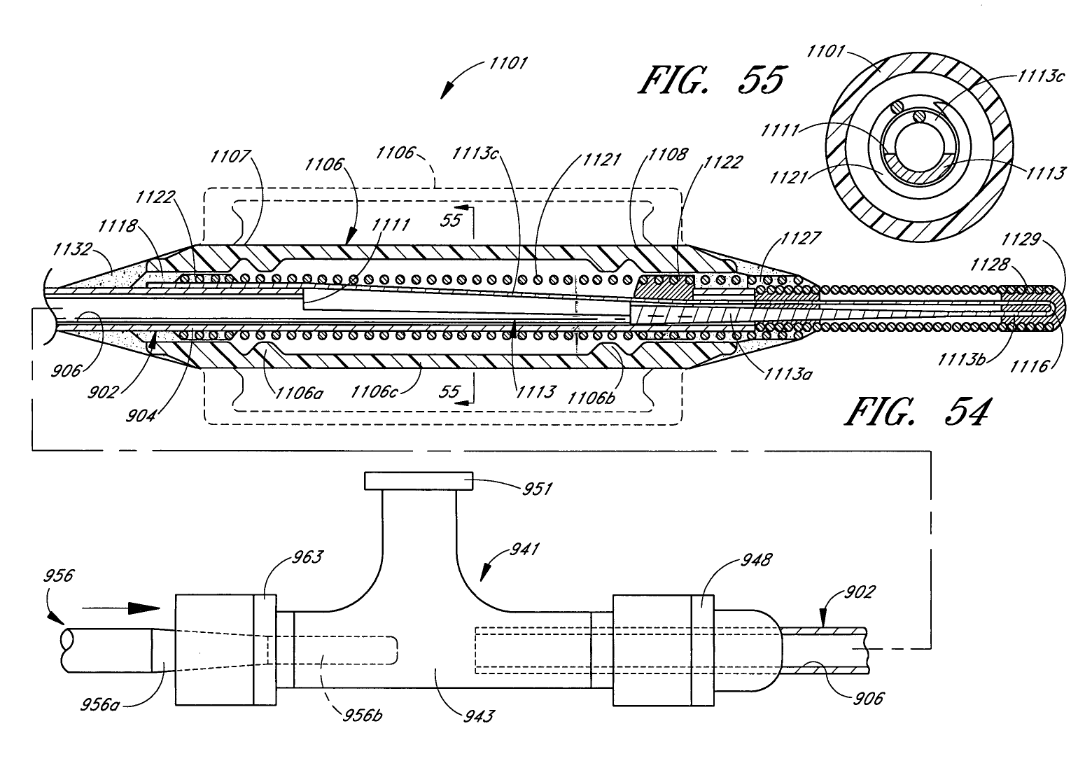

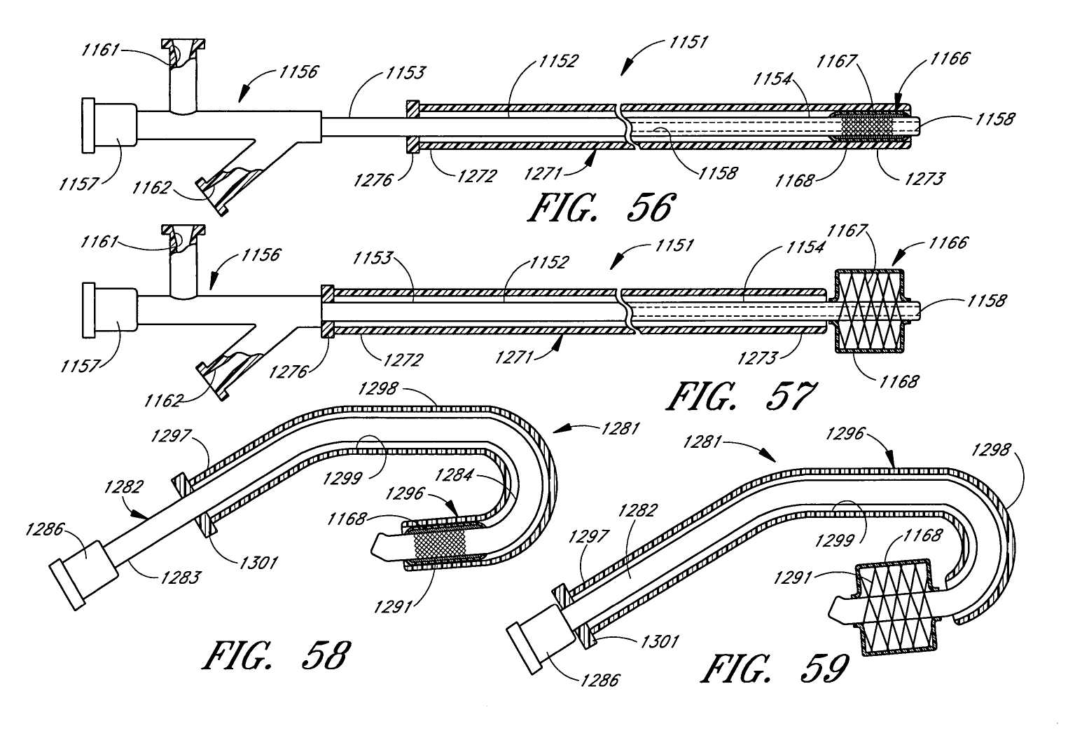

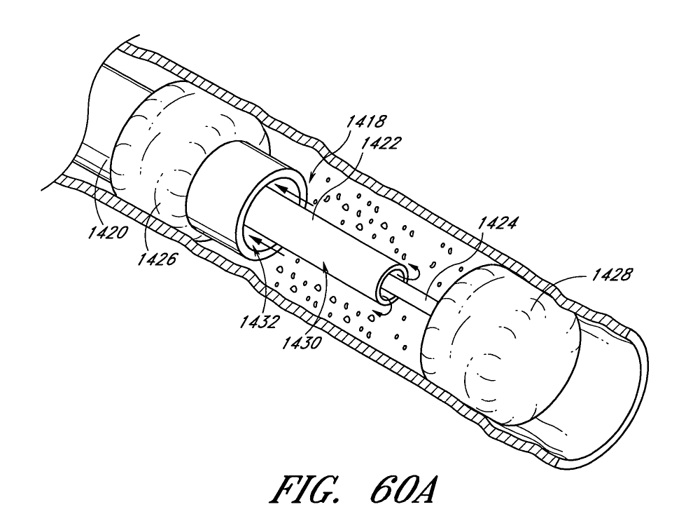

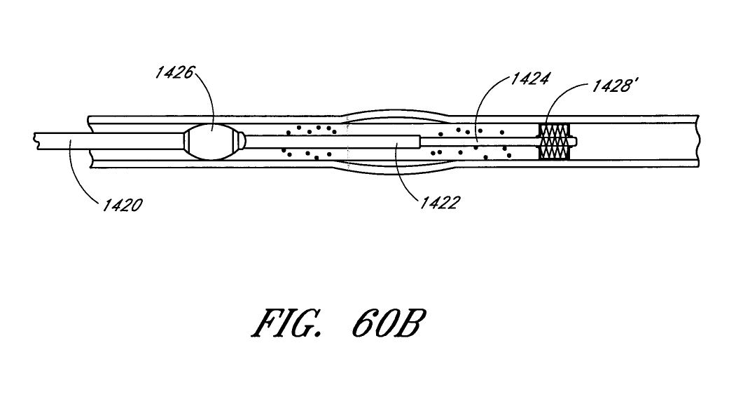

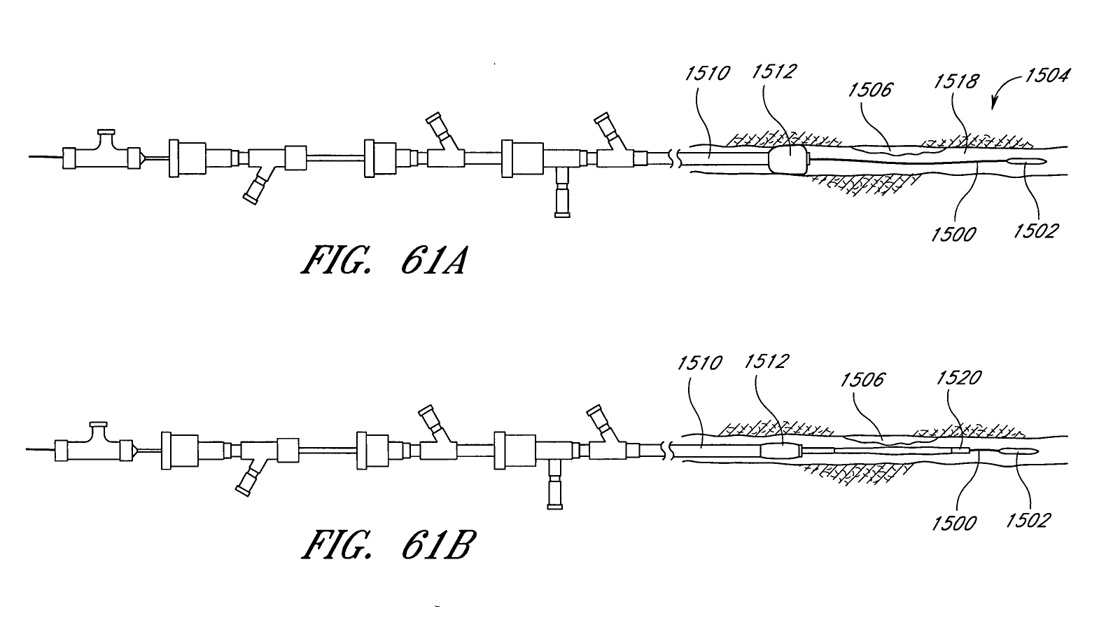

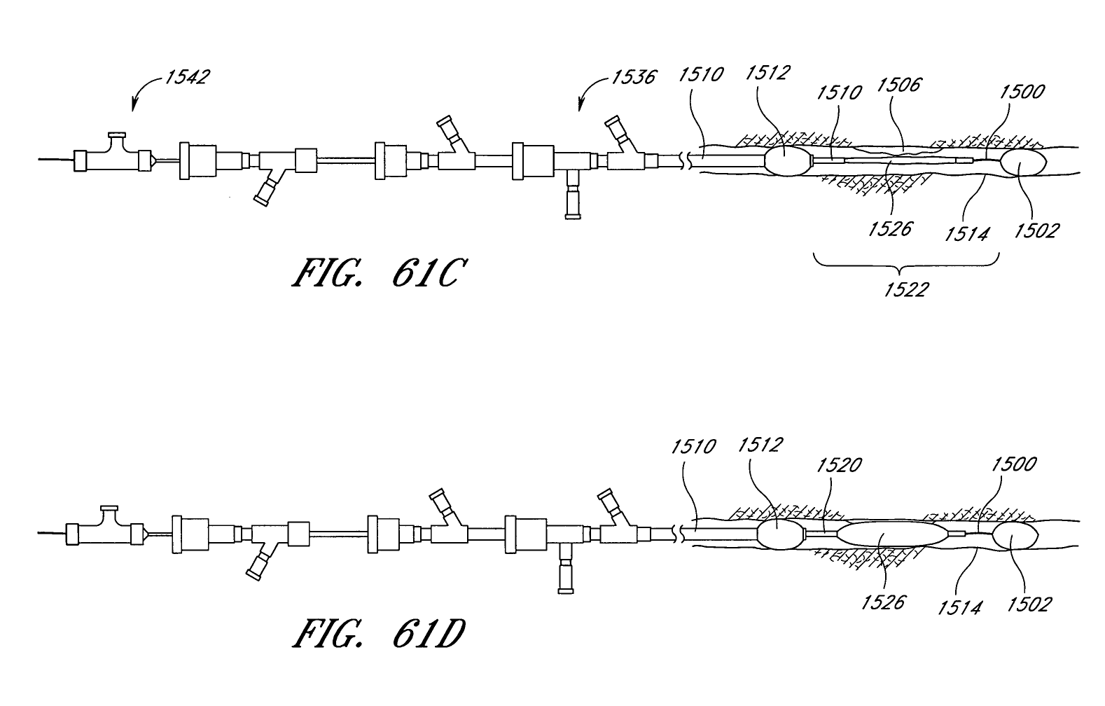

Operation and use of the apparatus of the present invention in performing a procedure in a carotid artery is shown in the cartoons in FIGS. 49-53. Let it be assumed that it is desired to perform a procedure with the apparatus of the present invention on a carotid artery in a patient, as for example common carotid 1041 which branches into an external carotid 1042 and an internal carotid 1043 and that there is a narrowing or a stenosis 1044 in the internal carotid 1043 near the bifurcation into the external and internal carotids 1042 and 1043. The main catheter 1031 can be introduced in the manner hereinbefore described with respect to a saphenous vein graft. For example it can be introduced through the femoral artery in the leg and then advanced into the aortic arch up into the common carotid 1041 until the occlusion balloon 869 carried thereby is near the bifurcation as shown in FIG. 49. The occlusion balloon 869 is then inflated to at least one atmosphere as shown in FIG. 50 to form a seal to occlude the common carotid 1041 and to temporarily stop the flow of blood to the face and brain of the patient through the common carotid 1041 and to provide a working space 1045 distal of the occlusion balloon 869. The inflation is accomplished by suitable means as for example a syringe 1046 secured to the balloon inflation fitting 887. Thereafter, a balloon-on-a-wire device 1031 of the type shown in FIG. 48 is introduced through the catheter port 1033 and advanced through the central lumen 857 of the main catheter 1031 after which the distal extremity is guided into the external carotid 1042 so it is disposed beyond the bifurcation. The occlusion balloon 911 carried by the distal extremity is then inflated by suitable means such as a syringe 1047 secured to the attachment 1036 to occlude the external carotid. As hereinbefore pointed out, the balloon 911 is an occlusion balloon that typically is inflated to a suitable occlusion pressure as for example one to two atmospheres.

Another balloon-on-a-wire device such as the balloon-on-a-wire device 901 is then introduced through the catheter port 1034 and advanced through the central passage or lumen 857 until it exits from the main catheter 1031 after which it is guided into the internal carotid 1043 past the stenosis 1044 so that the occlusion balloon 911 is distal of the stenosis 1044. The occlusion balloon 911 is then inflated as shown by the dotted lines in FIG. 50 by the use of a syringe 1048 secured to the inflation port carried by the removable valve attachment 941. Thus, the limits of the working space or chamber 1045 are defined by the occlusion balloons 869 and 911. As soon as the balloon 911 has been inflated, the balloon inflation lumen can be plugged in the manner hereinbefore described by the use of a plug mandrel 956 (see FIG. 51). It should be appreciated even though the guide wire 902 and the occlusion balloon 911 carried thereby may dislodge particles from the plaque forming the stenosis 1044, the dislodged particles will not travel to the brain because the common carotid supplying blood to the internal carotid 1043 has been occluded by the occlusion balloon 869.

The removable valve attachment 941 can then be removed in the manner hereinbefore described so that the proximal extremity of the guide wire 902 is free of obstructions as shown in FIG. 51. Thereafter the irrigation catheter 966 can be introduced over the guide wire 902 and thence into the port 1034 until its distal extremity extends beyond the distal extremity of the main catheter 1031. A therapeutic balloon catheter 1001 of the same type as hereinbefore described can then be introduced through the irrigation catheter 966. It should be appreciated that if desired, the therapeutic balloon catheter can be preloaded into the irrigation catheter 966 and the irrigation catheter 966 and the therapeutic balloon catheter 1001 can be introduced in unison. Assuming that the irrigation catheter 966 has been introduced first, the therapeutic balloon catheter 1001 is introduced through the irrigation catheter 966 until it extends beyond the distal extremity of the irrigation catheter 966 and is moved into the working space 1045 until the therapeutic balloon 1009 carried by the distal extremity thereof is in registration with the stenosis 1044. The therapeutic balloon 1009 is then inflated as shown in FIG. 51 by the use of an inflation/deflation device 1051 represented schematically by a syringe to a suitable therapeutic pressure to compress the plaque forming the stenosis 1044 to dilate the stenosis to increase the size of the flow passage through the stenosis 1044. The therapeutic balloon 1009 can then be deflated. In the event emboli 1004 are created as hereinbefore described by the passage of the therapeutic balloon 1009 through the stenosis, these emboli 1004 can be removed as shown in FIG. 52 by introducing a saline solution through the tube 1019 and into the irrigation port 973 of the irrigation catheter 966 to cause a saline solution to be discharged into the space formed between the two occlusion balloons 911 and 869. To achieve a more effective aspiration, the distal tip of the irrigation catheter 966 can be moved through the stenosis 1044 to just proximal of the occlusion balloon 911. Aspirate is removed through the aspiration port 889 through the use of the bulb 1021 and the collection bag 1024 to remove the saline solution carrying with it the emboli 1004 which may have been created and deposit the same in the collection bag 1024. This irrigation and aspiration can be carried on for a suitable period of time as for example 5 to 30 seconds after which the occlusion balloons 911 in both of the branches 1042 and 1043 can be deflated and the devices 901 and 1035 can be removed along with the catheter 1001 carrying the therapeutic balloon 1009. Similarly, the occlusion balloon 869 can be deflated to permit blood to flow into the common carotid 1041 and the external and internal carotids 1042 and 1043. Alternatively, the sequence of deflation of the balloons can be carried out in the manner hereinbefore described in connection with a vessel without a bifurcation.

In the event it is desired to deliver a stent into the dilated stenosis 1044, this can be accomplished by reinflating the occlusion balloon 869 and then reinflating the occlusion balloons 911 in both of the branches after which a balloon stent delivery catheter 1026 of the type hereinbefore described can be delivered over the guide wire 902 in the same manner as the therapeutic balloon catheter 966 and delivered into the desired location and then deployed in the dilated stenosis 1044. After the stent 1027 has been deposited and the balloon of the stent delivery catheter 1026 is deflated, the irrigation and aspiration procedures hereinbefore described can be repeated to remove any emboli within the space formed between the occlusion balloons 911 and 869. The stent delivery catheter 1026 can be removed. After a suitable period of irrigation and aspiration, as for example 5 to 30 seconds, the occlusion balloon 911 can be deflated after which the occlusion balloon 869 can be deflated and the balloon-on-a-wire devices 901 and 1035 removed along with the main catheter 852.

From the foregoing it can be seen that there has been provided a new and improved apparatus and a method for utilization of the same which makes it possible to carry out such stenosis opening procedures without the perfusion of blood. Complete stenosis procedures can be carried out in a period of time which is less than six minutes for each complete procedure. Even though blood flow is occluded during this period of time, this period of time is much less than the period of time, as for example 30 minutes, required for a conventional endoatherectomy. Thus, the procedures of the present invention can be carried out without endangering the patient, as for example the brain or the heart of the patient.

The desire to eliminate the use of a large guiding catheter for use with the main catheter 851 was hereinbefore discussed. Also, it was hereinbefore disclosed that the main catheter 851 can be inserted independently through a conventional sheath (not shown) in the femoral artery and thereafter a smaller conventional guiding catheter 986 is introduced through the main catheter so that its distal extremity 989 is in the vessel. In other procedures it may be desirable to carry this concept still further, i.e. eliminating the need for a large guiding catheter and also the need for a smaller guiding catheter to be advanced through the main catheter. To do this, it may be desirable to provide a distal extremity on the main catheter 851 which is shaped in a predetermined manner. For example, in the main catheter 851a shown in FIG. 29A there is provided in the distal extremity a conventional Judkins left shape and in the main catheter 851b shown in FIG. 29B there is provided in the distal extremity a conventional Judkins right shape. Other than the shaping of the distal extremities as hereinbefore described, the main catheters 851a and 851b are constructed in a manner very similar to the catheter 851 and are provided with occlusion balloons 869 as shown.

Since the main catheters 851a and 851b are relatively flexible, they can be inserted into the femoral artery and have their distal extremities guided into the desired locations with the catheter being selected for the appropriate bend to reach the desired location. With the main catheter having such capabilities, it is possible in connection with the present invention to advance the main catheter 851 into the desired location by the use of a balloon-on-a-wire device of the type hereinbefore described, or alternatively over a conventional guide wire. This makes it possible to eliminate the use of a guiding catheter and therefore substantially simplify the procedures of the present invention and reduce the costs of such procedures.

In connection with the irrigation catheter 966 hereinbefore described in FIG. 39, it should be appreciated as shown in FIGS. 39A and 39B that irrigation catheters 766a and 766b can be provided which have soft distal extremities to provide additional flexibility and trackability and thereby reduce trauma in vessels through which they are introduced. Thus in the irrigation catheter 966a shown in FIG. 39A, the main portion of the flexible elongate tubular member 967 which can be considered to be the shaft can have a greater stiffness than the distal portion 967a of the distal extremity. This can be readily accomplished by utilizing a plastic such as Pebax and Hytrel of various desired durometers. For example, the main shaft 967 can have a durometer ranging from 80-100 whereas the distal portion 967a can have a durometer ranging from 50-70. The cylindrical tip 967c with a rounded forward edge as shown is provided with a still lower durometer as for example 35-55 durometer. Thus it can be seen that there has been provided an irrigation catheter which has a very soft tip and which has a distal portion in the distal extremity which is very flexible to permit tracking and to reduce trauma.

In the irrigation catheter 966b shown in FIG. 39, the shaft 967 can have a durometer ranging from 80-100 whereas the portion 967a can have a durometer ranging from 60-70 and which has a portion 967b formed of the same durometer material that is inclined inwardly and distally to reduce the size of the opening for the passage or lumen 968 as shown. The tip 967 which can be formed of a low durometer as for example 35-55 durometer is mounted on the distal extremity 967b . In order to enhance the flow of irrigation fluid from the lumen 968 a plurality of holes 1057 is circumferentially distributed around the portion 967a to augment the flow of irrigation fluid other than through the passage 1056. The use of the embodiments 966a and 966b of the irrigation catheter is very similar to that hereinbefore described with the irrigation catheter 966 shown in FIG. 39. It should be appreciated that if differing stiffnesses are desired for the main catheters 851 and 1031, the same concepts as disclosed for the irrigation catheter 966 can be utilized by selecting materials having desired durometers for various portions of the catheters.

Another embodiment of the balloon-on-a-wire device is shown in FIGS. 54 and 55 in which the balloon-on-a wire device 1101 is in many respects very similar to the balloon-on-a-wire device 901 shown in FIG. 33 as hereinbefore described. The balloon-on-a-wire device 1101 consists of a flexible elongate member in the form of a guide wire 902 having proximal and distal extremities 903 and 904 with a lumen 906 extending therethrough. A removable valve attachment or fitting 941 is provided on the proximal extremity 1107. A plug mandrel 956 is carried by the removable valve attachment 941 for use in plugging the bore 906 when necessary. An elastomeric balloon 1106 is provided on the distal extremity 904 and is provided with proximal and distal extremities 1107 and 1108. The balloon 1106 has a suitable length as for example 10 millimeters and a suitable diameter when collapsed or deflated of 1 mm. In order that the balloon 1106 assume a generally rectangular shape as viewed in cross-section as shown in FIG. 54 with generally right angle comers, the balloon 1106 is provided with spaced-apart cylindrical regions 1106a and 1106b of greater thickness than an intermediate portion 1106c. For example, portions 1106a and 1106b can have a thickness of 0.006″ to 0.010″ and portion 1106c of 0.003″ wall thickness. Such a balloon when inflated will have a squareness as illustrated by the dotted lines in FIG. 54. This squareness of the balloon comers helps to assure that emboli will not become entrapped between the balloon and the vessel wall and thereby will not roll by the balloon as it is moved in the vessel.

An elongate slot 1111 is ground into the distal extremity of the guide wire 902 to a suitable depth which is in excess of one half of the diameter of the guide wire 902. The slot 1111 is in communication with the lumen 906 and opens into the interior of the balloon 1106. A tapered core wire 1113 is mounted in the distal extremity 904 of the guide wire 902. The core wire 1113 is provided with a portion 1113a which has a progressive decrease in diameter extending from the proximal extremity to a portion 1113b which is generally of a uniform diameter of a suitable size, as for example 0.003″ and is formed into a bend 1116 and extends proximally along the slot 1116 and proximally thereof where it is secured to the guide wire 902 by suitable means such as an adhesive 1118. A coil spring 1121 formed of a suitable material such as stainless steel or platinum extends over the slot 1111 and proximally and distally of the slot 1111 and is secured thereto by suitable means as solder 1122. Positioned in this manner, the coil 1121 generally circumscribes the inner circumference of the balloon 1106 and serves to protect the balloon 1106 from any sharp edges as for example sharp edges formed by the slot 1111 in the coil wire 902. A tip coil 1126 formed of a suitable radiopaque material such as a platinum or a platinum alloy is mounted over the distal extremity of the guide wire 902 and secured thereto by suitable means such as solder 1127. The distal extremity of the tip coil 1126 which may have a suitable length, as for example 3 mm, is bonded to the core wire 1113b by a solder 1128 which encloses the bend 1116 and provides a rounded forwardly protruding surface 1129. The distal extremity 1108 of the balloon 1106 is secured to the coils 1121 and 1126 by an adhesive 1131. Similarly, the proximal extremity 1107 of the balloon 1106 is secured to the guide wire 902 and the portion 1113b by an adhesive 1132.

The balloon-on-a-wire device 1101 can be utilized in the same manner as the balloon-on-a-wire device 901 hereinbefore described. It is believed that the balloon-on-a-wire device 1101 has several desirable features. For example the balloon 1106 is protected from any sharp edges by the coil spring 1121. The slot 1111, in addition to providing a means for inflating the balloon, also serves to provide a progressive weakening of the distal extremity of the guide wire 902 to impart additional flexibility to the distal extremity of the device.

By utilizing a balloon-on-a-wire constructions herein disclosed, it is possible to reduce the overall size of the apparatus for the procedures. In view of the fact that guide wires having a size of 0.014″ to 0.018″ are utilized in the present invention, many conventional therapeutic balloon devices can be utilized by advancing the same over such size guide wires. By the provision of removable valve attachments for the balloon-on-a-wire devices, it is possible to use such devices for providing the one or more balloons necessary for a procedure while at the same time making it possible to utilize such devices as guide wires after removing the removable valve attachments on the proximal extremities. This makes it possible to utilize conventional stent delivery catheters, ultrasound catheters and the like by advancing them over the already in place guide wires.

It should be appreciated that it may be possible to eliminate the use of the occlusion balloons 911 which are distal of the proximal balloon carried by the main catheter and distal of the stenosis, since blood flow is occluded during the time that the occlusion balloon 869 is inflated.

Another embodiment of a catheter apparatus incorporating the present invention for treating occluded vessels is shown in FIGS. 56 and 57. As shown therein, the catheter apparatus 1151 consists of a flexible elongate member 1152 similar to those hereinbefore described which is provided with proximal and distal extremities 1153 and 1154. A conventional adapter 1156 is mounted on the proximal extremity and is provided with a Tuohy-Borst fitting 1157 which is in communication with a large central lumen 1158 extending from the proximal extremity 1153 to the distal extremity 1154. An aspiration fitting 1161 is provided on the adapter 1156 as well as an irrigation fitting 1162, both of which are in communication with the central lumen 1158. However, it should be appreciated that if desired separate lumens can be provided in the flexible elongate member 1152 for both of the fittings 1161 and 1162.

Self-expanding sealing means 1166 is mounted on the distal extremity 1154. This self-expanding sealing means 1166 can take any suitable form. For example, as shown it can consist of a braided structure 1167 formed of a suitable shape memory material such as a nickel titanium alloy that will attempt to expand to a predetermined shape memory. Other than shape memory materials, other materials such as stainless steel, titanium or other materials can be utilized in the braid 1167 as long as they have the capability of expanding when the self-expanding seal means is released. Also it should be appreciated that the self-expanding seal means 1166 can be comprised of an absorbent material which when it absorbs saline or blood expands to form a seal. Such seals can be readily accomplished because it is only necessary to form a seal of approximately one atmosphere to prevent small particles from moving downstream.

In order to prevent abrasion of a vessel, it is desirable to cover the braided structure 1167 with a covering 1168 of a suitable material such as a polymer which extends over the braided structure 1167 and which moves with the braided structure 1167 as it expands and contracts. The polymer can be of a suitable material such as silicone, C-flex, polyethylene or PET which would form a good sealing engagement with the wall of the artery.

Means is provided for compressing the self-expanding sealing means 1166 so that the apparatus can be inserted into the vessel 981 and consists of an elongate sleeve 1271 having proximal and distal extremities 1272 and 1273 and a bore 1274 extending from the proximal extremity 1272 to the distal extremity 1273. A collar 1276 is mounted on the proximal extremity 1272 of the sleeve 1271 and is positioned near the adapter 1156. The collar 1276 serves as means for retracting the sleeve as shown in FIG. 57 to uncover the self-expanding sealing means 1166 after the catheter has been deployed to permit the self-expanding sealing means 1166 to expand and form a seal with the arterial vessel adjacent the stenosis to be treated.

Another embodiment of a catheter apparatus for treating occluded vessels incorporating the present invention is shown in FIGS. 58 and 59. As shown therein, the apparatus 1281 consists of a guiding catheter 1282 having proximal and distal extremities 1283 and 1284. As shown, the distal extremity 1283 is provided with a pre-formed bend of a conventional type. A conventional attachment 1286 is mounted on the proximal extremity 1283. Self-expanding seal means 1291 is mounted on the distal extremity 1284 and is of the type hereinbefore described in connection with the embodiments shown in FIGS. 56 and 57. A sleeve 1296 similar to the sleeve 1271 of the previous embodiment is provided in the present embodiment for encasing the self-expanding seal means 1291 and for releasing the same after it has been disposed in an appropriate position within a vessel adjacent the occlusion to be treated. Thus a sleeve 1296 is provided having proximal and distal extremities 1297 and 1298 and having a bore 1299 extending from the proximal extremity to the distal extremity which is sized so that it can receive the guide catheter 1282. It is provided with a collar 1301 on its proximal extremity which is adapted to be disposed outside the patient and which is adapted to be grasped by the physician for pulling the sleeve 1296 proximally to uncover the self-expanding seal 1291 after the apparatus has been deployed to permit the self-expansion of the sealing means 1291 to form a seal with the vessel wall is shown in FIG. 59.

In accordance with the hereinbefore described descriptions, it is apparent that the apparatus can be readily deployed and serve the same function as the main catheter. To accomplish this, the assembly 1281 can be introduced into the femoral artery and the distal extremity advanced into the desired location in the arterial vessel. After it has been properly positioned, the physician can retract the sleeve 1296 to permit the self-expanding seal means 1291 to expand and to form a seal with the wall of the arterial vessel to occlude the arterial vessel and interrupt the flow of blood in the vessel to provide a working space distal of the occlusion formed. This prevents small particles which may thereafter be dislodged from moving downstream. Since a central lumen is available, the therapeutic procedures hereinbefore described can be employed with the catheter apparatus shown in FIGS. 56, 57, 58 and 59.

Thus it can be seen that it has been possible to substantially reduce the complexity of the apparatus utilized in such procedures. This reduces the cost of the apparatus used therein as well as reducing the time required for performing such procedures making the procedures less costly.

E. Treatment Methods Described in U.S. Pat. No. 6,022,336

Referring to FIG. 60A, there is shown a schematic illustration of the catheter system of the present invention and the manner in which it forms an emboli containment chamber for efficient emboli removal. The catheter system, in this embodiment, comprises a three-catheter system, including an outer or main catheter 1420, an intermediate catheter 1422, and an inner or guidewire catheter 1424. This catheter system is shown schematically inserted within a relatively small vessel having a diameter d. As set forth above, the diameter d of the vessel may be as small 3 mm to 4 mm; although the present system can be efficiently utilized within vessels of larger diameter. An emboli containment chamber is formed between the outer 1420 and inner 1424 catheters, each of which in the preferred embodiment are provided with inflatable occlusion balloons 1426 and 1428. As noted above, the present invention is compatible with other types of occlusive devices, including those which permit perfusion and those which have other deployment mechanisms, such as filters, braids and the like. An embodiment with a distal occlusive filter 1428′ is shown in FIG. 60B. The present system is also compatible with containment chambers of variable length. Chambers of longer lengths contain a large volume of fluid and, thus, increase the time for emboli evacuation and/or increase the pressure differential (Δp) required to achieve desirable evacuation flow rates. Thus, containment chambers in the range of about 0.3 cc to 30 cc are preferable.

Although FIG. 60A illustrates the present catheter system deployed within a straight vessel, it will be understood that the principles of the present invention also include other vessel configurations, including branches vessels. In such cases, a third or even additional occlusive devices may be used in order to contain the emboli and form a working chamber. Such occlusive devices could be mounted on additional inner catheters similar to the one illustrated in FIG. 60A, or on a single inner catheter having itself two branches, or otherwise. FIG. 60A illustrates an important feature of the present invention in which the catheters 1420, 1422, 1424 are telescoped within one another. Thus, the inner catheter 1424 is relatively small in outer diameter and fits within the inner diameter of the intermediate catheter 1422 and can, in some applications, serve as a guidewire therefor. Likewise, the outer diameter of the intermediate catheter 1422 fits within the inner diameter of the outer or main catheter 1420. The catheters 1420, 1422, and 1424 thus form inner and outer pathways, 1430 and 1432 between the inner 1424 and intermediate 1422 catheters and the intermediate 1422 and outer 1424 catheters, respectively. It is through these pathways 1430, 1432 that irrigation or aspiration may be performed. Advantageously, in the present system, irrigation can be performed through the inner pathway 1430 and aspiration through the outer pathway 1432, or vice versa. As explained below in more detail, irrigation refers to the injection of fluid through one of the pathways into the containment chamber in order to generate an evacuation flow rate. Fluid, together with emboli, are evacuated through the other pathway, being assisted by the aspiration pressure which is in reality a suction or negative pressure. It is this pressure differential over some length within the chamber which generates the evacuation fluid flow.

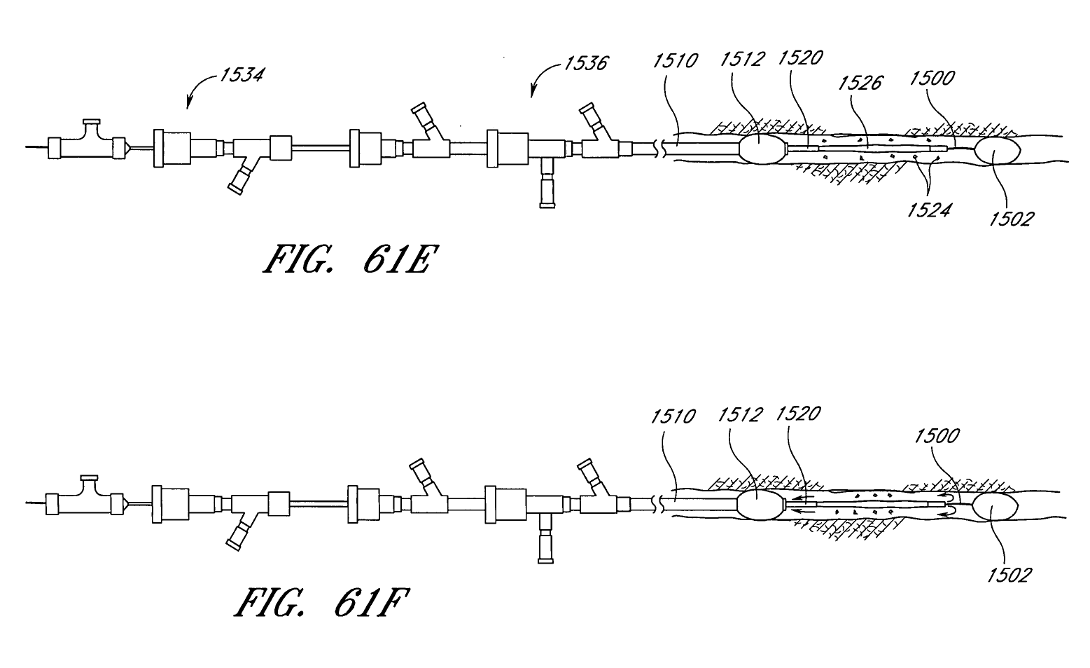

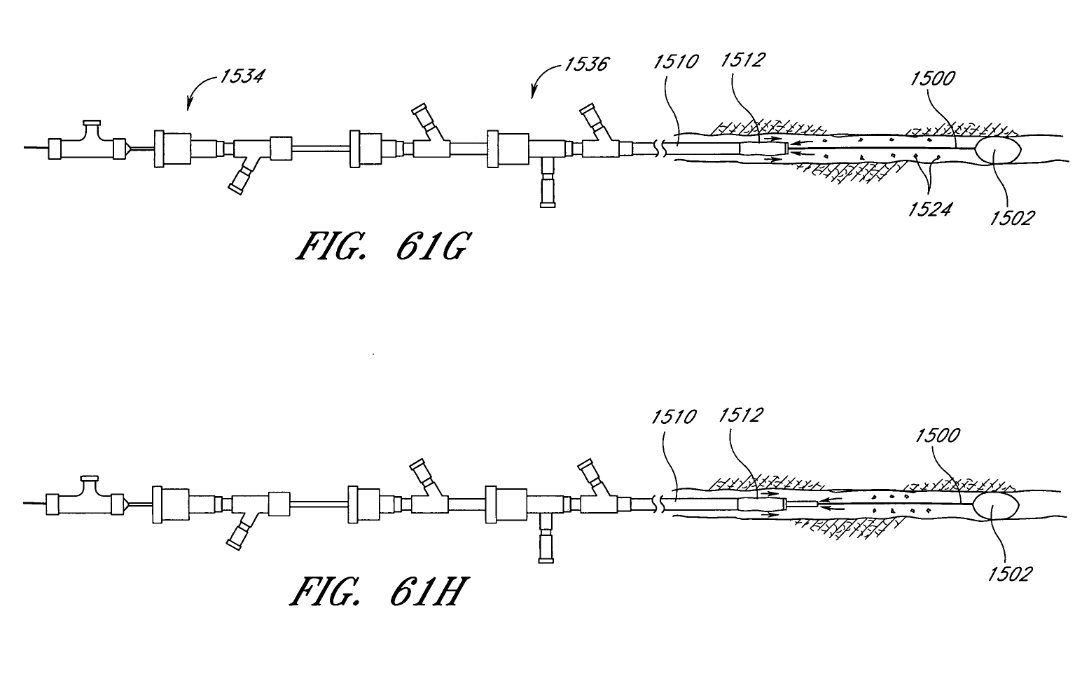

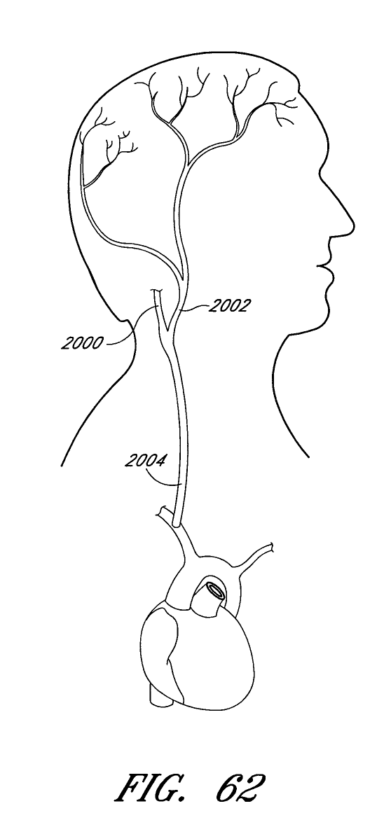

The operation and use of the emboli containment system utilizing the catheters of the present invention for treating occluded vessels will now be described in connection with an occlusion formed by a stenosis in a carotid artery, as illustrated in FIGS. 61A-H. It should be noted that this application is merely exemplary, and that the method of the present invention can be used in other blood vessels in the body as well. The word “proximal” as used herein refers to the portion of the catheter closest to the end which remains outside the patient's body, while “distal” refers to the portion closest to the end which is inserted into the body.

A guiding catheter (not shown) is first introduced into the patient's vasculature through an incision in the femoral artery in the patient's groin. The guide catheter is advanced through the artery into the aorta of the heart of the patient and into the ostium of the carotid artery to be treated, where it remains throughout the procedure if needed. Fluoroscopy is typically used to guide the catheter and other devices to the desired location within the patient. The devices are typically marked with radiopaque markings to facilitate visualization of the insertion and positioning of the devices.

Referring now to FIG. 61A, a main catheter 1510 having a distal attached occlusive device 1512, in this example an inflatable balloon, is advanced into the ostium of the carotid artery and into the lumen 1518 of the vessel 1514. The main catheter 1510 with the occlusive device 1512 thereon is advanced until the device 1512 is just proximal to the stenosis 1506. The device is activated, i.e. the balloon 1512 is then inflated, to occlude the vessel 1514. The inner catheter, in this example a guidewire 1500, having an occlusive device 1502, in this example an inflatable balloon, at its distal end 1504 is next delivered through the main catheter 1510. The occlusive device 1502 is positioned just distal to the occlusion 1506. The occlusive device is activated, i.e., the balloon 1502 is inflated to create an isolated chamber within the vessel which surrounds the occlusion. The balloons 1502, 1512 are each progressively inflated until they engage the side wall of the vessel 1514 to occlude the lumen 1518. Preferably, the distance between the proximal end of the occlusive device on the guidewire 1504 and the distal tip of the occlusive device on the main catheter 1516 should be approximately 5-10 cm. Advantageously, the present invention allows for the creation of a treatment and containment chamber whose length can be easily adjusted to isolate a specific area within a blood vessel. As soon as both balloons 1502, 1512 are inflated, a working space 1522 is provided between the balloons 1502, 1512, so that therapeutic procedures can be undertaken to remove or reduce the occlusion 1506 in the space between the balloons 1522, without risk of unwanted particles or emboli escaping into the blood stream. The inner catheter 1500 and the main catheter 1510 with their attached distal occlusive devices 1502, 1512 are therefore used to create a chamber 1522 surrounding the occlusion 1506, and act to contain the emboli and debris 1524 resulting from the treatment of the occlusion 1506 as illustrated in FIG. 61C.

Alternatively, a guide catheter or angiography catheter can first be delivered to the site of the occlusion. The inner catheter is inserted through the guide or angiography catheter, and positioned within the patient. The guide or angiography catheter is removed, and the main catheter is inserted over the inner catheter into position proximal to the occlusion. The occlusive device at the distal end of the main catheter is activated, the occlusive device on the inner catheter is put into position distal to the occlusion and activated, and the procedure continues as described above.

Alternatively, the main catheter can be delivered directly to a position just proximal to the occlusion, without use of a guide or angiography catheter. The inner catheter is then delivered through the main catheter as described above.

In another alternative embodiment of the present invention, the inner catheter can be delivered first through the guide catheter. The occlusive device on the distal end of the inner catheter is positioned distal to the occlusion. The main catheter is introduced over the inner catheter and advanced into the ostium of the carotid artery and into the lumen of the vessel. The main catheter is advanced until the balloon is just proximal to the occlusion. The intermediate catheter is then delivered into the chamber to provide appropriate therapy. The occlusive devices on the distal ends of the inner and main catheters are activated, to create a treatment and isolation chamber surrounding the occlusion. This method can be used when the physician determines that the risk of crossing the occlusion prior to activation of the proximal occlusive device is minimal.

Referring now to FIG. 61B, once the chamber has been created around the occlusion, an intermediate catheter 1520 is delivered to the site of the occlusion 1506. In the example illustrated in FIGS. 61A-F, the intermediate catheter 1520 is a therapy catheter having an angioplasty balloon on its distal end. The intermediate catheter 1520 is delivered to the sit, of the occlusion 1506 as shown in FIG. 61B.

The term “therapy catheter” is meant to include any of a number of known devices used to treat an occluded vessel. For example, a catheter carrying an inflatable or mechanically activated balloon for use in balloon angioplasty, as is used in this example, can be delivered to dilate the stenosis. Thermal balloon angioplasty includes the use of heat to “mold” the vessel to the size and shape of the angioplasty balloon. Similarly, an intravascular stent can be delivered via a balloon catheter and deployed at the site of the stenosis to keep the vessel open. Cutting, shaving, scraping, or pulverizing devices can be delivered to excise the stenosis in a procedure known as atherectomy. A laser or ultrasound device can also be delivered and used to ablate plaque within the vessel. Various types of rheolitic devices could be used. Various thrombolytic or other types of drugs can be delivered locally in high concentrations to the site of the occlusion. It is also possible to deliver various chemical substances or enzymes via a catheter to the site of the occlusion to dissolve the obstruction. A combined aspiration and therapy catheter can also be used. The term “therapy catheter” encompasses these and other similar devices.

Referring now to FIG. 61D, after the balloons 1502 and 1512 are properly inflated, and the therapy catheter 1520 in place, therapy begins. For emboli containment systems featuring balloon dilatation treatment, it is desired to compress the plaque or material forming the 1506 to provide a larger passageway through the vessel. Thus, a balloon angioplasty catheter 1520 is positioned such that the distal end with the balloon 1526 thereon is at the site of the occlusion 1506. When the balloon 1526 has been properly positioned within the 1506, the balloon 1526 is inflated with a suitable inflation medium, as for example a radiopaque liquid. The angioplasty balloon 1526 can be inflated to the desired pressure to cause compression of the plaque of the occlusion 1506 against the sidewall of lumen 1514 by the application of appropriate inflation pressure, as shown in FIG. 61D. As in conventional angioplasty procedures, the balloon 1526 can be formed of a non-elastic relatively non-compliant material so that appropriate pressures, such as 10-15 atmospheres, can be created within the balloon to apply compressive forces to the vessel 1514 without danger of rupturing the vessel 1514. It should be appreciated that the non-elastic capabilities can also be achieved by a composite elastic material.

After appropriate therapy has been performed and the occlusion 1506 has been removed or lessened using any of the methods and apparatus described above, the therapy balloon 1526 is deflated as illustrated in FIG. 61E. A source of irrigation fluid (not shown) is connected to the adaptor 1534 located at the proximal end of the therapy catheter 1520, and a source of aspiration pressure (not shown) is connected to an adaptor 1536 located at the proximal end of the main catheter 1510, as illustrated in FIG. 61F. Preferably, the source of irrigation fluid is a bag of normal saline, typically used in intravenous infusion. The source of aspiration pressure is preferably a syringe. After the source of irrigation and aspiration are connected, irrigation and aspiration are begun. Irrigation fluid is provided through the inner pathway between the therapy catheter 1520 and the guidewire 1500, while aspiration is provided through the outer pathway between the therapy catheter 1520 and the main catheter 1510 as shown by the small arrows in FIG. 61F. Of course it is to be understood that irrigation fluid could be provided through the outer pathway while aspiration is provided through the inner pathway. In either case, suitable pressures are provided to ensure that the change in pressure inside the chamber does not damage the vessel. The change in pressure as fluid flows into and out of the chamber should not exceed about 50 psi. Suitable pressures range from approximately −10 to −30 in-Hg aspiration pressure, and about 5 to 30 psig irrigation pressure. Note that these pressures are measured at the proximal end of the catheters.

In an alternative embodiment not shown, after therapy has been performed to remove or reduce the occlusion the therapy catheter is removed from the emboli containment system, and an irrigation catheter is delivered to the emboli containment chamber. The irrigation catheter is inserted through the main catheter lumen. The main lumen of the irrigation catheter can ride over the inner catheter, or the inner catheter can be positioned in a separate lumen adjacent to the main lumen. The distal end of the irrigation catheter is positioned just proximal the distal occlusion balloon, preferably approximately 1-2 cm from the balloon. As noted above, the irrigation and main catheter are sized such that the irrigation catheter can pass through the main catheter lumen and the annulus or outer pathway between the main catheter lumen and the irrigation catheter is large enough to allow aspiration of the blood and debris through it. Irrigation fluid is provided through the inner pathway between the inner catheter and the irrigation catheter. Alternatively, an aspiration catheter, a combined irrigation/aspiration catheter, or similar debris removing device such as a rheolitic device, can be used as the intermediate catheter. In this embodiment of the invention, the aspiration catheter is delivered in the same manner as described above for the irrigation catheter. Aspiration then occurs through the inner pathway, while irrigation is provided through the outer pathway.

Once the desired catheters are properly positioned, irrigation and aspiration are performed. The irrigation fluid and aspiration pressure are delivered in such a way as to ensure that the change of pressure within the chamber is below about 50 psi to avoid damaging the vessel. The irrigation fluid, preferably normal saline solution, is preferably delivered at a pressure of from about 5 psi to about 50 psi; 5 psi is preferred. The aspiration pressure is preferably between about −5 and −30 in-Hg, and more preferably is about −20 in-Hg. Again, these pressures are measured from at the proximal end of the catheters. The irrigation and aspiration can be delivered simultaneously, continuously, or delivery can be pulsed, or one can be delivered continuously while the other is delivered in a pulsed fashion. The user can determine the best method of delivery to provide optimized flow, turbulence, and clearance within the chamber.

Referring again to FIG. 61F, it is preferable that the inflation pressure within the distal occlusion balloon 1502 is maintained at a level greater than the pressure in the chamber and the jet created by irrigation to avoid the leakage of fluid and debris past the distal occlusion balloon 1502. Similarly, the inflation pressure in the proximal occlusion balloon 5232 should be maintained at a level greater than the pressure in the chamber and the aspiration pulsation to avoid having fluid aspirated from behind the balloon 1512 and possibly aspirating the balloon 1512 itself. Again, the irrigation and aspiration pressures provided are such that the change in pressure during fluid flow into and out of the vessel does not damage the vessel. The change in pressure is preferably no greater than about 50 psi.

In another embodiment of the present invention, after the therapy catheter is removed, the aspiration catheter is delivered such that its distal end is positioned approximately 1-2 cm from the distal occlusive device. The proximal occlusive device is then deactivated, to allow blood flow into the chamber. This blood flow is used as irrigation flow. The blood, acting as irrigation fluid, is aspirated together with particles and debris through the aspiration catheter. This eliminates the need for a separate source of irrigation fluid. In this embodiment, it is preferred that the blood flow rate in the vessel is greater than about 100 cc/min, and flow rates of 60-80 cc/min are preferred. This method is illustrated in FIG. 61G.

In yet another embodiment, illustrated in FIG. 61H, after the therapy catheter is removed, the proximal occlusive device is deactivated, allowing blood flow into the chamber. The blood, acting as irrigation fluid, is aspirated together with particles and debris through the opening in the main catheter. This eliminates the need for a separate aspiration catheter and a separate source of irrigation fluid, thereby reducing the time necessary to complete the procedure.

Aspiration and irrigation are continued until particles and debris 1524 are removed from the chamber 1522, then the irrigation, aspiration, or the therapy catheter 1520, is removed. First the distal 1502 and then the proximal 1512 occlusion balloons are deflated, and the guidewire 1500 and main catheter 1510 are removed. Finally, the guide catheter is removed, and the incision in the patient's femoral artery is closed.

F. Treatment Methods Described in U.S. application Ser. No. 09/270,150

The preferred embodiments of the present invention provide improved methods for containing and removing emboli resulting from plaque, thrombi or other occlusions. The preferred methods are particularly advantageous for use in the carotid artery and other arteries above the aortic arch. The preferred methods may be used, for example, in the treatment of a stenosis or an occlusion which has a length and a width or thickness resulting in at least partial occlusion of the vessel's lumen. Thus, the preferred methods are effective in treating both partial and substantially complete occlusions of arteries. It is to be understood that “occlusion” as used herein, includes both complete and partial occlusions, stenoses, emboli, thrombi, plaque, and any other substance which at least partially occludes the lumen of the artery. Although the methods disclosed herein are described with specific reference to the carotid arteries, they can be applied to other vessels as well, particularly bifurcated vessels.

As illustrated in FIG. 62, the common carotid artery 1610 is located in the neck and branches off into the internal carotid 1612, and the external carotid 1614 arteries. The internal carotid artery 1612 supplies blood to the brain, while the external carotid artery 1614 supplies blood to the head and face. The preferred methods of the present invention will be described for the treatment of an occlusion within the internal carotid artery. It is to be understood that this method can be used on other arteries as well.

Generally, the preferred methods are adapted for the percutaneous treatment, containment and removal of occlusions within the carotid arteries or other arteries above the aortic arch. In one of these methods, a main catheter having an occlusive device on its distal end is first delivered to the common carotid artery, proximal to the site of the occlusion. It should be noted that, as used herein, “proximal” refers to the portion of the apparatus closest to the end which remains outside the patient's body, and “distal” refers to the portion closest to the end inserted into the patient's body. The occlusive device is activated to stop the downstream flow of blood. Collateral pressures from the Circle of Willis and other vessels keep the blood flow in the direction of the main catheter, preventing any emboli from moving downstream. In another embodiment, a main catheter without an occlusive device on its distal end, or a main catheter having an occlusive device which is not deployed, is delivered to the common carotid artery, proximal to the site of the occlusion.

In either case, an inner catheter having an occlusive device on its distal end is delivered through the main catheter and across the site of the occlusion. Alternatively, a detachable occlusive device can also be used. In either case, the occlusive device is activated at a site distal to the occlusion.

In some cases, a second inner catheter is used to provide a third occlusive device. One inner catheter is delivered to the internal carotid artery, while the other inner catheter is delivered to the external carotid artery. When activated, the three occlusive devices completely isolate the area surrounding the occlusion to be treated.

A therapy catheter is then delivered to the site of the occlusion to treat the occlusion. Such treatment includes, but is not limited to, balloon angioplasty, thermal balloon angioplasty, delivery of an intravascular stent, atherectomy, or radiation treatment.

In one embodiment of the present invention, once therapy is complete, an irrigation catheter is delivered into the working area to provide irrigation fluid. Alternatively, anatomical irrigation can be used, as explained below. Aspiration of the area surrounding the treated occlusion is begun using either the main catheter or a separate aspiration catheter. Blood flow is allowed into the working area to be aspirated by deactivating the occlusive devices on the main and/or inner catheters. This helps to irrigate the area and ensure the removal of particles and debris from the artery.

In another embodiment of the present invention, the need for a separate irrigation catheter and irrigation fluid are eliminated. In the context of removing plaque, thrombi or other blockages from blood vessels, separate irrigation fluid is generally provided through an irrigation catheter to the site of treatment. It has been discovered that the patient's own blood can be used as irrigation fluid, without the need for delivery of a separate irrigation catheter and irrigation fluid.

Although the patient's own flow of blood can provide an irrigation source, situations sometime arise where providing separate irrigation fluid is desired. In such cases a separate catheter is introduced into the patient after the therapy catheter is removed and is delivered within close proximity to the occlusive device. Once the catheter is delivered proximal to the occlusive device, the area is first aspirated through the catheter. By delivering the catheter close to the occlusive device a turbulence is created freeing debris from the edge of the occlusive device and other areas where it may be trapped. The debris is then aspirated from the patient. Following aspiration, irrigation fluid is provided if desired to flush any remaining particles and debris from the internal carotid.

Main Catheter

In the preferred methods, a main or guide catheter is first introduced into the patient's vasculature. This catheter is used to guide the insertion of other catheters and devices to the desired site. A guide catheter (e.g., 9 F) or a long sheath (e.g., 7 F) may be used as the main catheter. If the guide catheter is not sufficiently stiff, then an angiography catheter may be positioned inside the guide catheter, and both the guide catheter and the angiography catheter can be delivered on a guidewire. (The term guidewire is used broadly herein to include elongate members (such as hollow or tubular members) made of metal as well as other materials, such as plastic.) Once the guide catheter is properly positioned, the angiography catheter can be removed. In some embodiments of the present invention, the main catheter has an occlusive device on its distal end. The occlusive device can be an inflatable balloon, filter, expandable braid or other mechanical occlusive device. The occlusive device should be capable of preventing the migration of particles and debris from the working area, either through total or partial occlusion of the vessel. Note that the occlusion of the vessel need not be complete, and that substantial occlusion of the vessel may be sufficient. The catheter should be sized so as to slidably receive the inner, therapy and intermediate (irrigation and/or aspiration) catheters inserted therethrough.

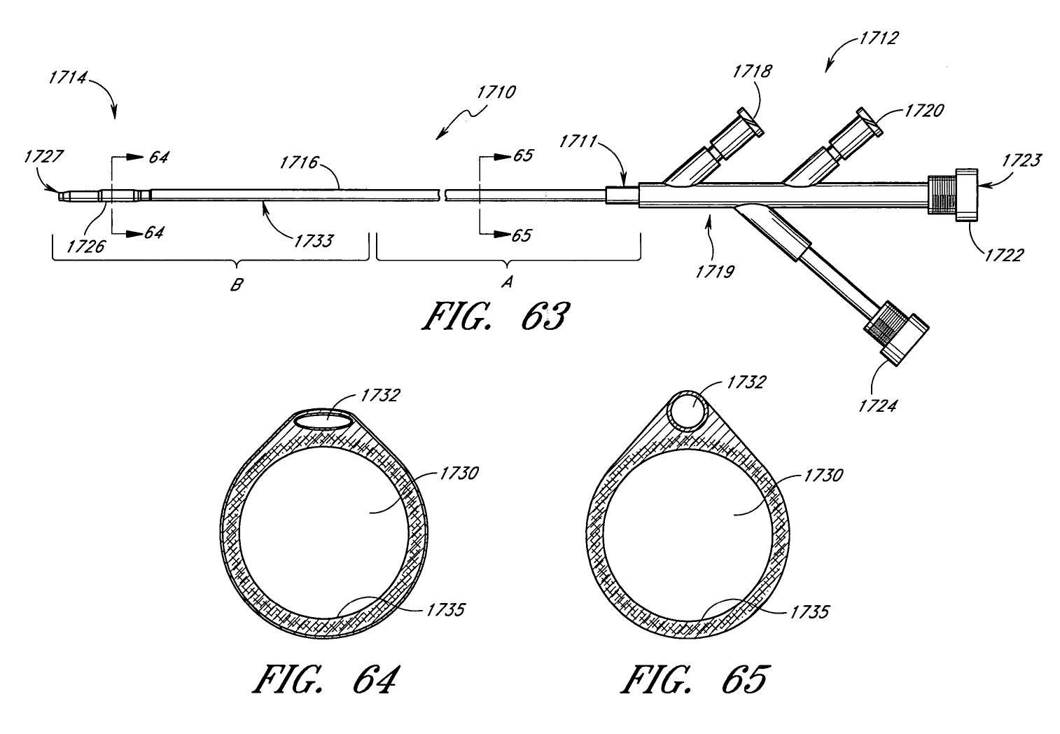

FIG. 63 illustrates a side view of a catheter which can be used as the outer or main catheter of the present system. Catheter 1710 generally comprises an elongate flexible tubular body 1716 extending between a proximal control end 1712 and a distal functional end 1714. The tubular body 1716 has a main lumen 1730 which extends between the ends 1712 and 1714. The main lumen 1730 terminates in a proximal opening 1723 and a distal opening 1727. A smaller inflation lumen 1732, configured in a side-by-side relationship with the main lumen 1730, extends along the length of the tubular body 1716, and terminates within an occlusive device such as an occlusion balloon 1726 mounted on the distal end 1714 of the catheter 1710, as described below. The inflation lumen 1732, illustrated in FIGS. 64 and 65, is in fluid communication with the occlusion balloon 1726, such that fluid passing through the inflation lumen 1732 may be used to inflate or deflate the balloon 1726. The proximal end of the inflation lumen can terminate at one of the ports 1722, 1724 on the proximal end of the catheter 1710.

A control manifold 1719 is provided at the proximal end 1712 of the catheter 1710. The control manifold 1719 is generally provided with a number of ports to provide access to the catheter lumen 1730. For example, for the embodiment depicted in FIG. 63, the control manifold 1719 is provided with a catheter end-access port 1722 and a catheter side-access port 1724, to provide an introduction point for the insertion of other catheters into the lumen 1730. Ports 1722 and 1724 are preferably provided with standard Touhy Borst connectors, although other types of connectors may be used. An inflation port 1718, in fluid communication with the small inflation lumen 1732, is further provided on the manifold 1719 for attachment of devices to inflate or deflate the occlusion balloon 1726. The manifold 1719 is also provided with an irrigation/aspiration port 1720 which is in fluid communication with the lumen 1730, for attachment of devices to provide irrigation fluid or aspiration pressure. Other embodiments of the main catheter 1710 may feature more or less ports, depending upon the number of lumens in the catheter and the desired functionalities of the catheter.

The manifold 1719 is preferably formed out of hard polymers or metals, which possess the requisite structural integrity to provide a functional access port to the catheter lumen, such as for balloon inflation or delivery of irrigation fluid and/or aspiration pressure. In one preferred embodiment, the manifold 1719 is integrally formed out of polycarbonate. Of course, any suitable material may be used to form the manifold 1719, including acrylonitrile butadiene styrene (ABS).

As illustrated in FIG. 63, an inflatable balloon 1726 is mounted on the distal end 1714 of the catheter 1710. The inflatable balloon 1726 will function as an occlusion balloon, to prevent blood and debris from passing through the blood vessel distal to the balloon 1726. Thus, the inflatable balloon 1726 is preferably able to expand to fit a variety of different blood vessel diameters. Accordingly, it is preferred that the inflatable balloon 1726 have a compliant expansion profile, tending to increase in radial diameter with increasing inflation pressure. To achieve this, the balloon 1726 may be made out of materials which impart such expansion characteristics, including elastomeric materials such as latex or irradiated polyethylene. In one preferred embodiment, the inflatable balloon 1726 is formed out of a material comprising a block copolymer of styrene-ethylene-butylene-styrene, sold under the trade name C-FLEX. Non-compliant balloons, such as those made from PET can also be used.

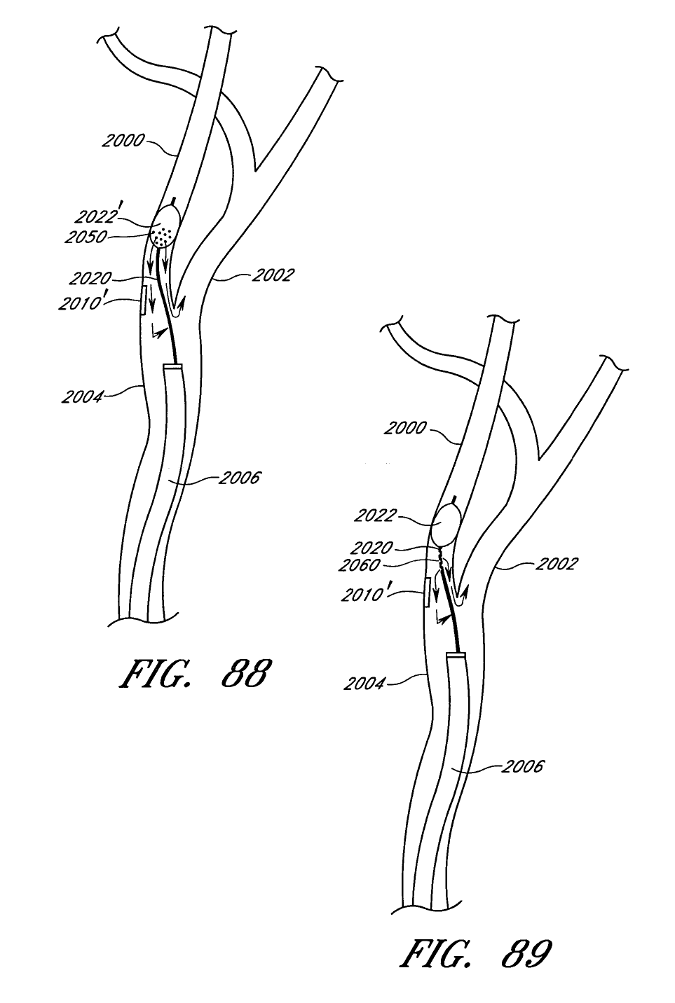

Alternatively, as illustrated in FIGS. 80-81, the main catheter 2006 does not include a distal occlusive device, or the distal occlusive device on the main catheter is not used.

Inner Catheter

An inner catheter or guidewire having an occlusive device on its distal end is preferably made of metals such as stainless steel or nitinol, or plastics or composites. The preferred methods can be effectively carried out using any of a number of guidewires or catheters that perform the function of occluding the vessel and allowing for the slidable insertion of various other catheters and devices. The term “catheter” as used herein is therefore intended to include both guidewires and catheters with these desired characteristics.

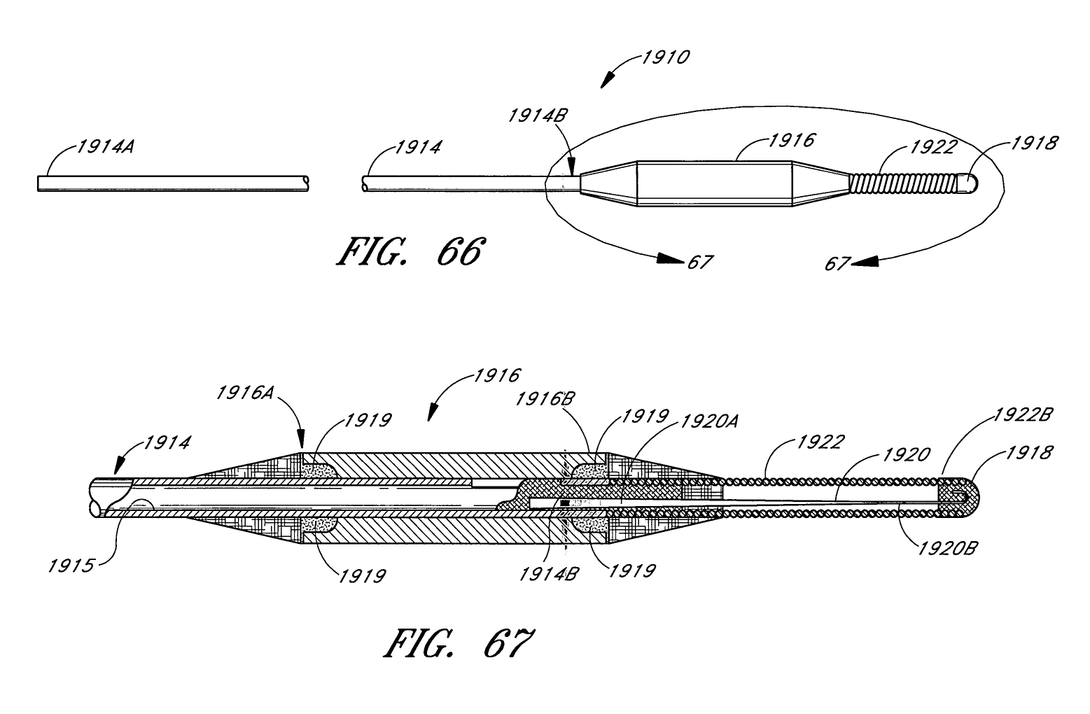

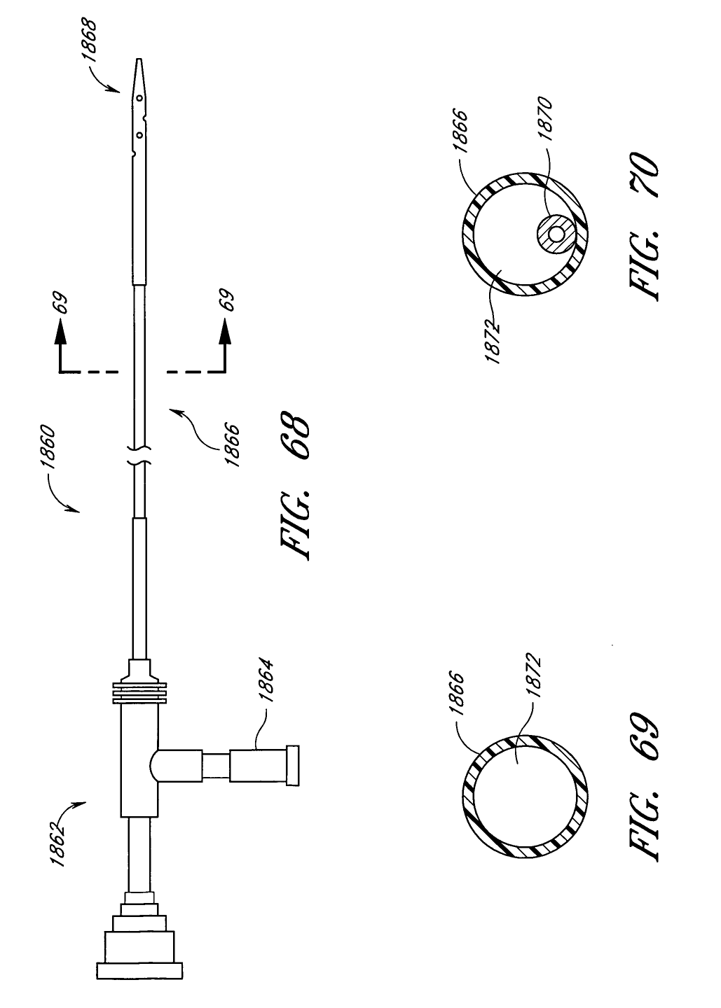

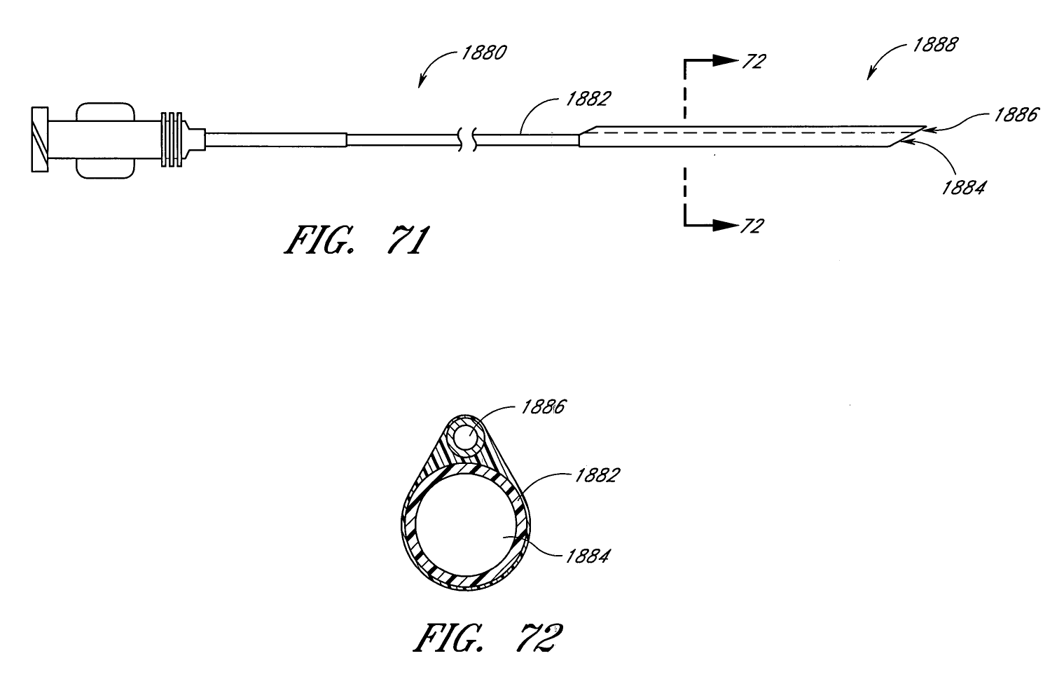

A preferred inner catheter is illustrated in FIGS. 66 and 67. The catheter apparatus 1910 is generally comprised of four communicating members including an elongated tubular member 1914, an inflatable balloon member 1916, a core-wire member 1920 and a coil member 1922. The catheter apparatus 1910 is preferably provided with an outer coating of a lubricous material, such as TEFLON.

The body member 1914 of the catheter apparatus 1910 is in the form of hypotubing and is provided with proximal and distal ends 1914A and 1914B as well as an inner lumen 1915 extending along the tubular member 1914. The balloon member 1916 is coaxially mounted on the distal end 1914B of the tubular member 1914 by suitable adhesives 1919 at a proximal end 1916A and a distal end 1916B of the balloon member 1916 as in the manner shown in FIG. 67. The core-wire member 1920 of the catheter 1910 may be comprised of a flexible wire 1920. The flexible wire 1920 is joined by adhesives, soldering, brazing or crimping at a proximal end 1920A of the flexible wire 1920 to the distal end 1914B of the tubular member 1914 as in the manner show in FIG. 67.

Preferably, the proximal end 1920A of the flexible wire 1920 has a transverse cross sectional area substantially less than the smallest transverse cross-sectional area of the inner lumen 1915 of the tubular member 1914. In the preferred embodiment, the flexible wire 1920 tapers in the distal end 1920B to smaller diameters to provide greater flexibility to the flexible wire 1920. However, the flexible wire may be in the form of a solid rod or a ribbon or combinations thereof.

As shown in FIG. 67, the distal end 1920B of the flexible wire 1920 is secured to a rounded plug 1918 of solder or braze at the distal end 1922B of the coil member 1922. The coil member 1922 of the catheter 1910 may be comprised of a helical coil 1922. The coil member 1922 is coaxially disposed about the flexible wire 1920, and is secured to the flexible wire 1920 by soldering, brazing or adhesives at about the proximal end 1920A of the flexible wire 1920 as in the manner shown in FIG. 67.

The balloon member 1916 is preferably a compliant balloon formed of a suitable elastic material such as a latex or the like, but can be made of non-compliant materials as well. The flexible coil 1922 is preferably formed of a wire of platinum based alloys or gold. The flexible core-wire 1920 and the tubular member 1914 are preferably formed of a nickel-titanium alloy or stainless steel.

Once the inner catheter has been properly positioned inside the carotid artery at a point distal to the occlusion, the occlusive device at the distal end of the inner catheter is actuated to occlude the vessel distal to the existing occlusion to create a working area. When a detachable occlusive device is used, the occlusive device is positioned at a point distal to the occlusion to be treated, and activated to occlude the artery. It is to be understood that the stenosis or occlusion could be in a discrete location or diffused within the artery. Therefore, although placement of the occlusive device is said to be distal to the stenosis or occlusion to be treated, portions of the diffused stenosis or occlusion may remain distal to the occlusive device.

Therapy Catheter

After the area surrounding the occlusion has been isolated, a therapy catheter then is delivered to the site of the occlusion. The term “therapy catheter” is meant to include any of a number of known devices used to treat an occluded vessel. For example, a catheter carrying an inflatable balloon for use in balloon angioplasty can be delivered to dilate the occlusion. Thermal balloon angioplasty includes the use of heat to “mold” the vessel to the size and shape of the angioplasty balloon. Similarly, an intravascular stent can be delivered via a balloon catheter and deployed at the site of the occlusion to keep the vessel open. Cutting, shaving, scraping or pulverizing devices can be delivered to excise the occlusion in a procedure known as atherectomy. A laser or ultrasound device can also be delivered and used to ablate plaque in the vessel. Thrombectomy devices can be used, as can rheolitic devices, and devices which create a venturi effect within the artery. Various thrombolytic or other types of drugs can be delivered locally in high concentrations to the site of the occlusion. It is also possible to deliver various chemical substances or enzymes via a catheter to the site of the stenosis to dissolve the obstruction. The term “therapy catheter” encompasses these and similar devices.

Aspiration and Irrigation Catheters

After the therapy has been performed and the occlusion has been treated, the working area may be aspirated to remove fluid and debris. Aspiration can be provided through the main catheter if desired. A source of negative pressure is attached at the proximal end of the main catheter, and fluid and debris are aspirated through the main catheter's main lumen. Alternatively, an aspiration catheter or similar debris removing device can be delivered to the working area to remove particles and any other debris. The term “aspiration catheter” includes any device which creates an area of fluid turbulence and uses negative pressure to aspirate fluid and debris, and includes thrombectomy catheters, rheolitic devices and those devices which create a venturi effect within the vessel. Thus, it is possible that a single catheter is used as both the therapy catheter and the aspiration catheter.

An aspiration catheter particularly suited for use with the preferred methods is illustrated in FIG. 68. The catheter 1860 includes an adapter 1862 and a seal at its proximal end. The catheter 1860 further includes an aspiration port 1864 to which a source of negative pressure is attached. The aspiration catheter further comprises a long hollow shaft 1866 having a distal end 1868. The distal tip 1868 can/include a radiopaque marker to aid in locating the tip 1868 during insertion into the patient, and is preferably soft to prevent damage to the patient's vasculature.

The aspiration catheter illustrated in FIG. 68 is an over-the-wire catheter. As seen in FIG. 69, the catheter shaft 1866 is hollow. During insertion of the aspiration catheter 1860, the proximal end of a guidewire 1870 is inserted into the distal end of the aspiration catheter 1868, and the aspiration catheter 1860 is slidably advanced over the guidewire 1870, which is positioned inside the hollow lumen 1872 of the aspiration catheter 1860. The position of the guidewire 1870 relative to the shaft 1860 of the aspiration catheter 1860 is illustrated in FIG. 70, but of course, can vary. For this type of aspiration catheter 1860, a very long guidewire 1870, generally around 1900 centimeters in length, is used to facilitate the insertion of the aspiration catheter 1860 over the guidewire 1870.

Alternatively, the aspiration catheter 1880 can be of a single operator design, as illustrated in FIGS. 71-72. The catheter 1880 has an adapter and an aspiration port at its proximal end. Like the over-the-wire aspiration catheter 1860 the single operator aspiration catheter 1880 further comprises a long hollow shaft 1882 having a distal end 1888. The distal tip 1888 can include a radiopaque marker to aid in locating the tip 1888 during insertion into the patient, and is preferably soft to prevent damage to the patient's vasculature. At the distal end of the shaft 1888, a guidewire lumen 1886 is attached. This lumen 1886 provides a separate lumen, apart from the main aspiration lumen 1884 of the catheter 1880, for the insertion of the guidewire. This guidewire lumen 1886 can be as short as 5 centimeters or longer. As illustrated in FIG. 72, during delivery of the aspiration catheter 1880, the proximal end of the guidewire is inserted into the distal end of the guidewire lumen 1886, and the guidewire lumen 1886 is slidably advanced over the guidewire. Unlike the over-the-wire catheter 1860 described above, only a short segment of the single operator aspiration catheter 1880 rides over the guidewire, and the guidewire remains in the guidewire lumen 1886 and does not enter the aspiration lumen 1884 of the aspiration catheter 1880. With the single operator system 1880, the long guidewire used with the over-the-wire catheter 1860, and the extra operator needed to handle it, are not required.

Although the guidewire lumen 1886 is shown in FIG. 71 as being located only on the distal end 1888 of the shaft of the aspiration catheter 1880, the lumen 1886 can also be made to extend the entire length of the shaft 1880 if desired. In both embodiments, the aspiration lumen 1884 is advantageously left completely unobstructed to provide more efficient aspiration. The guidewire lumen 1886 can also include a slit in the outside wall of the lumen to facilitate faster and easier insertion and removal of the guidewire through the side wall of the lumen.

In another embodiment not shown, the aspiration catheter can be configured such that the therapy catheter can be inserted through the lumen of the aspiration catheter. The lumen is made large enough to accommodate the desired therapy catheter. This allows the aspiration catheter and the therapy catheter to be delivered into the patient at the same time. When therapy is complete, the therapy catheter is removed while the aspiration catheter remains in place. This eliminates the need to separately deliver the aspiration catheter after removal of the therapy catheter, saving valuable time.

In yet another embodiment, also not shown, the therapy catheter can be built over the aspiration catheter. For example, a dual or triple lumen catheter having a dilatation balloon at its distal end can be used. One lumen is used to inflate the dilatation balloon to be used for angioplasty, while the second lumen is used for aspiration. The third lumen is used as a guidewire lumen. Alternatively, the aspiration catheter can be designed to deploy a stent within the occluded artery, or could include an atherectomy device on its distal end. These designs allows a single combined aspiration catheter and therapy catheter to be delivered into the patient. When therapy is complete, aspiration is carried out without the need to first remove the therapy catheter or separately deliver an aspiration catheter.

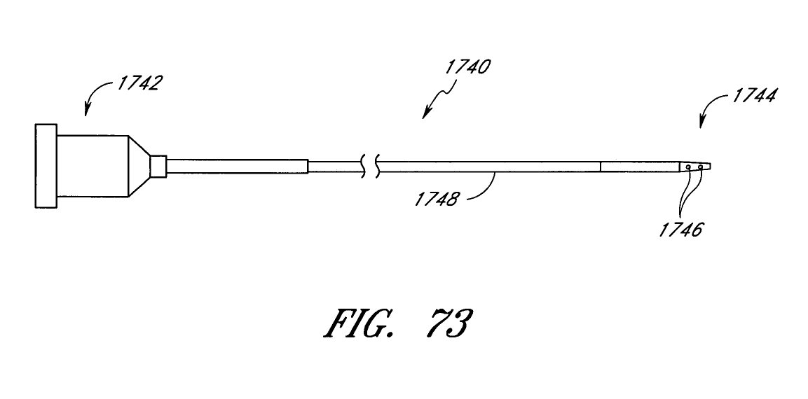

FIG. 73 is a side view of an irrigation catheter 1740 or aspiration catheter which may be utilized in the preferred methods. It should be understood that when an irrigation catheter is used, aspiration occurs through the outer pathway between the irrigation and main catheters, while irrigation occurs through the irrigation pathway. Similarly, when an aspiration catheter is used, aspiration occurs through the aspiration catheter while irrigation occurs through the pathway between the aspiration and main catheters. Irrigation fluid is supplied under pressure at the proximal end of the catheter 1742 and delivered through the side holes 1746 and through the distal end of the catheter 1744. Alternatively, aspiration can be provided at the proximal end of the catheter 1742 and fluid and debris aspirated through the side holes 1746 and through the distal end of the catheter 1744. The catheter 1740 can be about 125 centimeters in length and constructed from a plastic material such as HYTREL tubing or high density polyethylene (HDPE) or PEBAX (Atochem, France). In order to achieve a softer distal section, the durometer of the tube 1748 material is reduced in the distal section to about 1755 whereas that of the proximal section 1742 is higher, such as about 1780. Proximal valves and fittings which are well known in the art can be mounted on the catheter 1740 of FIG. 73.

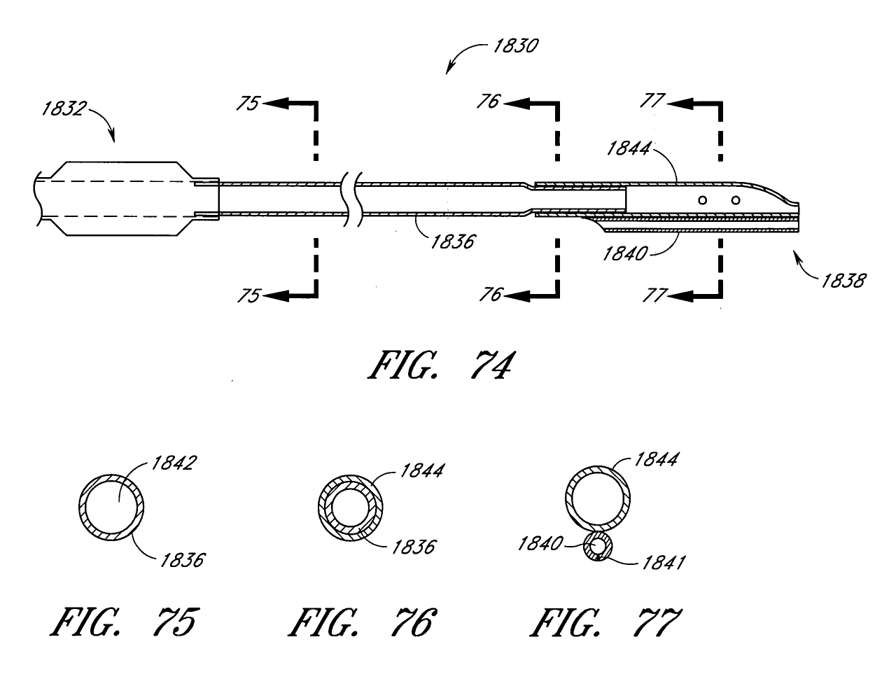

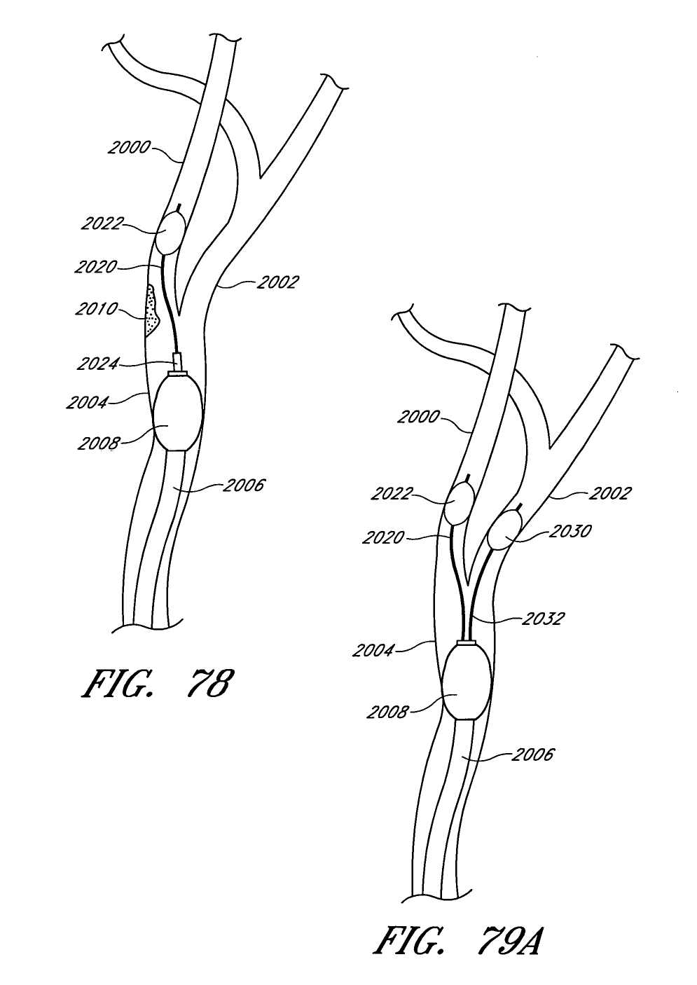

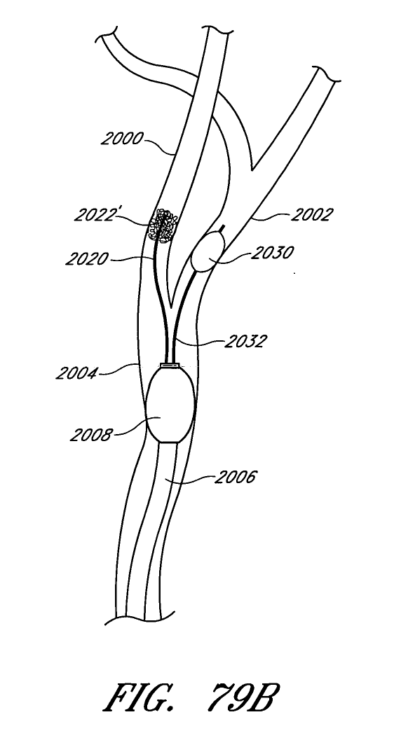

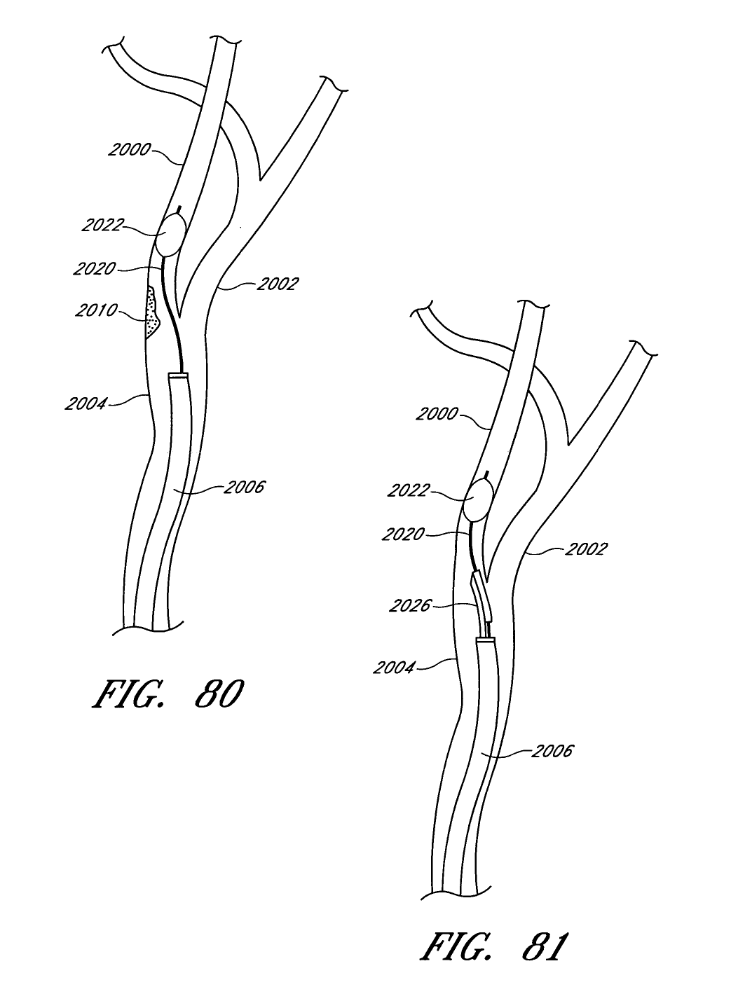

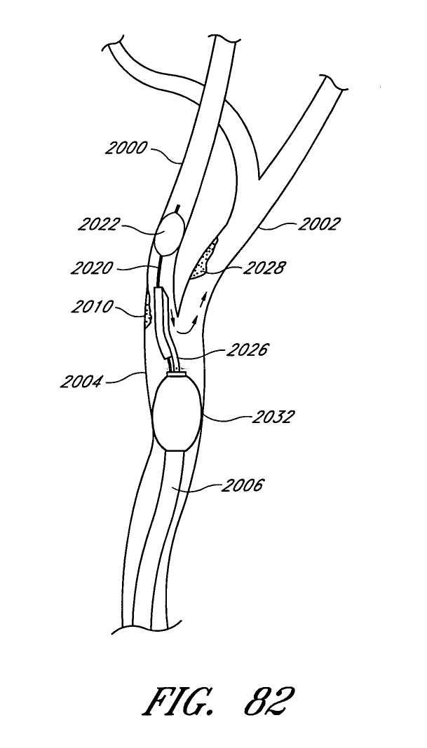

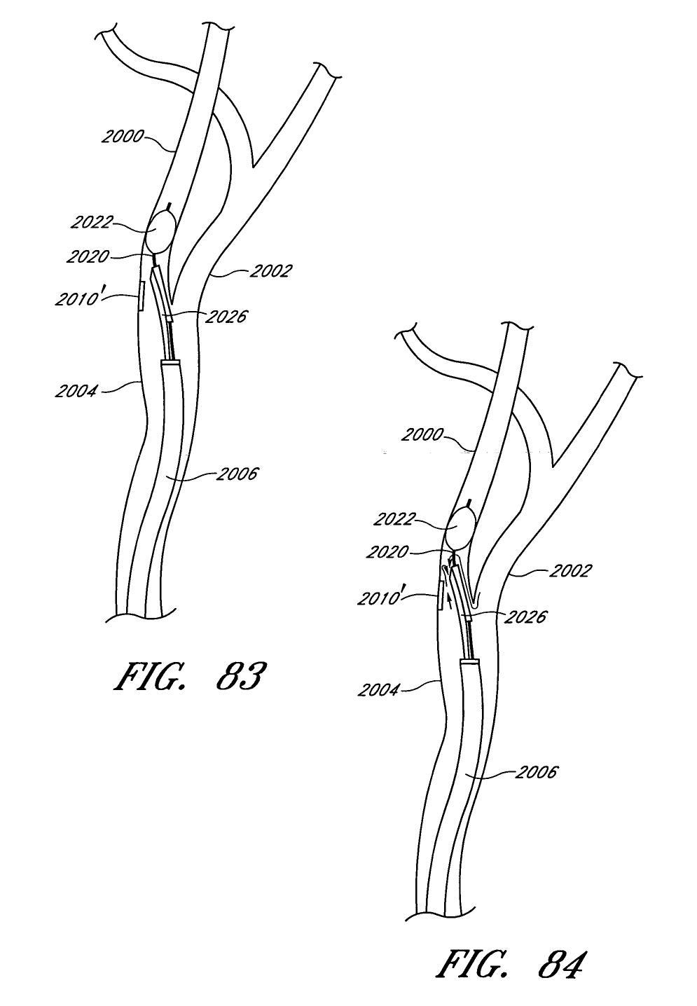

FIGS. 74-77 illustrate another type of irrigation or aspiration catheter 1830, a single operator catheter, which can be used in the present system. In the case of the irrigation catheter, irrigation is through the inner pathway and aspiration is through the outer pathway. If the catheter is used for aspiration, aspiration is through the inner pathway and irrigation is through the outer pathway. As shown in FIGS. 74-77, the catheter 1830 has an adaptor 1832 on its proximal end. This single operator catheter 1830 further comprises a long tubular body 1836 having a distal end 1838. The distal tip 1838 can include a radiopaque marker to aid in locating the tip 1838 during insertion into the patient, and is preferably soft to prevent damage to the patient's vasculature. At the distal end of the shaft 1838, an inner catheter lumen 1840 is attached. This lumen 1840 provides a separate lumen, apart from the main irrigation or aspiration lumen 1842 of the catheter 1830, for the insertion of the inner catheter, and has an inner diameter sized to received the inner catheter. In a preferred embodiment, the inner diameter of the lumen is about 0.016″ to about 0.020″, and more preferably is about 0.019″. This inner catheter or guidewire lumen can be as short as 5 centimeters, but can extend 30 centimeters or longer in a proximal direction. During delivery of the catheter 1830, the proximal end of the inner catheter is inserted into the distal end of the inner catheter lumen 1840, and the lumen 1840 is slidably advanced over the inner catheter. Only a short segment of the single operator catheter 1830 rides over the inner catheter, and the inner catheter remains in the lumen 1840 and does not enter the main lumen 1842 of the catheter 1830.

Although the inner catheter lumen 1840 is shown in FIG. 74 as being located only on the distal end 1838 of the shaft of the catheter 1836, the lumen 1840 can also be made to extend the entire length of the shaft 1836 if desired. In both embodiments, the main lumen 1842 is advantageously left completely unobstructed to provide more efficient irrigation or aspiration. As seen in FIG. 77, the inner catheter lumen 1840 can also include a slit 1841 or weakened area in the outside wall of the lumen 1840 along the entire length of the lumen 1840 to facilitate faster and easier insertion and removal of the inner catheter through the side wall of the lumen 1840. By inserting and removing the inner catheter through the side wall of the lumen 1840 on the catheter 1836, the need to remove adapters and attachments from the proximal end prior to slidably advancing or removing the catheter 1836 over the inner catheter is eliminated. It should be understood that this slit 1841 or weakened area through which the inner catheter can be inserted and removed can exist on the intermediate catheter regardless of whether the catheter is used for irrigation, aspiration, therapy or some other purpose.

In another embodiment, not shown, the irrigation and aspiration are conducted through a multi lumen catheter. In this embodiment, a single catheter is used. The catheter includes at least two separate lumens; one lumen is used for aspiration and has a source of negative pressure attached at the proximal end, while a second lumen is used to provide irrigation and has a source of irrigation fluid attached at the proximal end.

Preferred Methods

A. Dual Balloon System

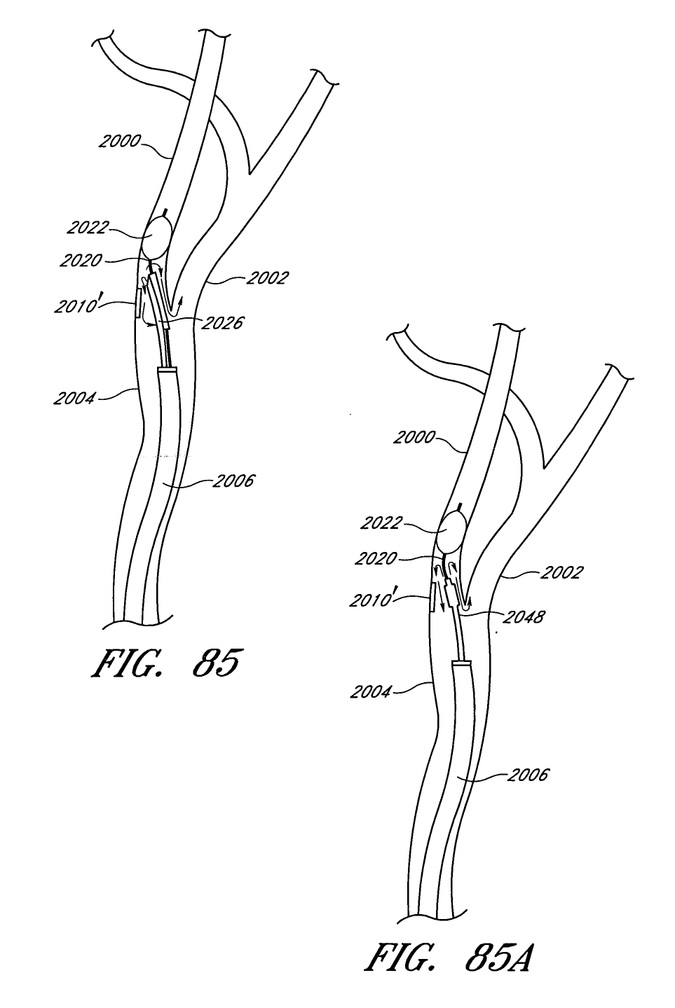

FIG. 78 illustrates the removal of plaque and any associated thrombi from the internal carotid artery 2000. It should be noted that this method is merely exemplary, and that occlusions in other locations, such as within the external carotid 2002, common carotid 2004 artery, or other arteries above the aortic arch, may be treated.

A main catheter or guide catheter 2006 is introduced into the patient's vasculature through an incision in the femoral artery in the groin of the patient, or through direct access to the arteries in the neck (e.g., jugular access, in which case the catheters do not need to be as long as in the case of femoral access). The main catheter 2006 has a lumen sized to receive other catheters and devices, and can be used to guide the insertion of these other catheters and devices. The main catheter 2006 is guided through the vasculature until it reaches the common carotid artery 2004, where it can remain in place throughout the procedure. Fluoroscopy is typically used to guide the main catheter 2006 and other devices to the desired location within the patient. The devices are frequently marked with radiopaque markings to facilitate visualization of the insertion and positioning of the devices within the patient's vasculature.

Once the main catheter 2006 is in place, with its occlusive device 2008 at a position proximal to the occlusion 2010, the occlusive device 408 is activated. Downstream blood flow is effectively stopped, and blood flow coming from collateral blood vessels distal to the occlusive device prevents the downstream migration of any free particles. In this example, the occlusive device 2008 is an inflatable balloon. The balloon is inflated to occlude the common carotid artery 2004.

Next, an inner catheter or guidewire 2020 having an occlusive device 2022 at its distal end is delivered through the main catheter 2006 into the internal carotid artery 2000 and past the site of the occlusion 2010. Alternatively, a detachable occlusive device can be deployed at the site distal to the occlusion, and the delivery device removed. In this example, the occlusive device 2022 is also an inflatable balloon. The balloon is inflated to occlude the internal carotid artery at a site distal to the occlusion 2010. It should be understood that the occlusion within the artery can be in a discrete location or diffused within the vessel. Therefore, although placement of the distal occlusive device is said to be distal to the occlusion to be treated, portions of the diffuse occlusion may remain distal to the occlusive device.

A working area is therefore created between the two occlusive devices 2008, 2022 surrounding the occlusion 2010. A therapy catheter (not shown) is then delivered. The therapy catheter can be any of a number of devices, including a balloon catheter used to perform angioplasty, a catheter which delivers a stent, a catheter for delivering enzymes, chemicals, or drugs to dissolve and treat the occlusion, an atherectomy device, a thrombectomy device, a rheolitic device, a device which creates a venturi effect within the artery, or a laser or ultrasound device used to ablate the occlusion.

Once the desired therapy is performed, the therapy catheter is withdrawn from the patient's body and an aspiration catheter 2024 is delivered through the main catheter 2006, preferably over the inner catheter or guidewire 2020. The aspiration catheter 2024 rides over the guidewire 2020 with the guidewire 2020 inserted through the aspiration lumen of the catheter 2024. Alternatively, a single operator type aspiration catheter can be used, in which only a portion of the aspiration catheter rides over the guidewire, which is inserted into a separate guidewire lumen. FIG. 78 illustrates the treatment site after the over-the-wire aspiration catheter 2024 is inserted into the internal carotid artery 2000.

After the aspiration catheter 2024 is in place, aspiration is begun. A source of negative pressure is connected to the aspiration catheter 2024 at its proximal end. A preferred source of negative pressure is any container containing a fixed vacuum, such as a syringe, attached to the proximal end of the aspiration catheter 2024 at the aspiration port. A mechanical pump or bulb or any other appropriate source of negative pressure can also be used, including the creation of a venturi effect within the blood vessel. The difference between the existing pressure within the vessel and the aspiration or negative pressure within the vessel should not exceed about 50 psi. If too much aspiration is applied, the change in pressure in the vessel will be too great and damage may occur to the vessel itself.

Prior to aspiration, simultaneous with aspiration, or after aspiration is begun, the proximal occlusive device 2008 is deactivated to allow blood flow into the area The blood flow into the area provides irrigation fluid which creates turbulence and facilitates the removal of particles and debris. Preferably, the anatomical irrigation pressure provided is approximately 1-1.5 psi, and the blood flow into the area is at least 10 cubic centimeters/min and more preferably about 60-80 cubic centimeters/min. In a preferred embodiment, the proximal occlusive device is then reactivated, and the distal occlusive device is deactivated. This allows blood flow into the working area from the distal end. Following aspiration, the distal occlusive device is reactivated. This method of alternately deactivating and reactivating the occlusive devices acts to contain and direct the emboli to an area within the working area where they will be aspirated. Particles are initially contained between the two occlusive devices. When the proximal occlusion device is deactivated, blood flow forces particles and debris toward the distal end of the working area. The working area is aspirated, and the occlusive device reactivated. When the distal occlusive device is deactivated, blood flow forces particles and debris back toward the proximal end of the working area, where they are then aspirated. The steps of deactivating and reactivating the occlusive devices and aspirating the working area can be repeated as often as desired, until the working area is substantially free of particles and debris.

When the deactivating and reactivating of the occlusive devices and aspiration steps are complete, the aspiration catheter is removed, and the occlusive devices are deactivated. The main and inner catheters are also removed from the patient.

As described above, the aspiration catheter can be sized such that it can receive the therapy catheter within its lumen. In this case, the aspiration catheter and the therapy catheter are delivered into the artery together. When therapy is complete, the therapy catheter is removed while the aspiration catheter remains in place. When aspiration is complete, the aspiration catheter, inner catheter and main catheter are removed from the patient's body. Delivering the aspiration catheter and therapy catheter together saves time, which is critical during these types of procedures.

In yet another embodiment, aspiration takes place through the lumen of the inner catheter or guidewire. The occlusive device on the inner catheter is positioned distal to the occlusion, and the occlusive device is activated to at least partially occlude the vessel. The therapy catheter is delivered and therapy performed. A source of negative pressure is provided at the proximal end of the inner catheter, and aspiration occurs through openings located at the distal end of the catheter just proximal to the occlusive device. This eliminates the need for a separate aspiration catheter, and the need to remove the therapy catheter prior to aspiration. Again, this saves time, which is critical during these types of procedures.

B. Triple Balloon System

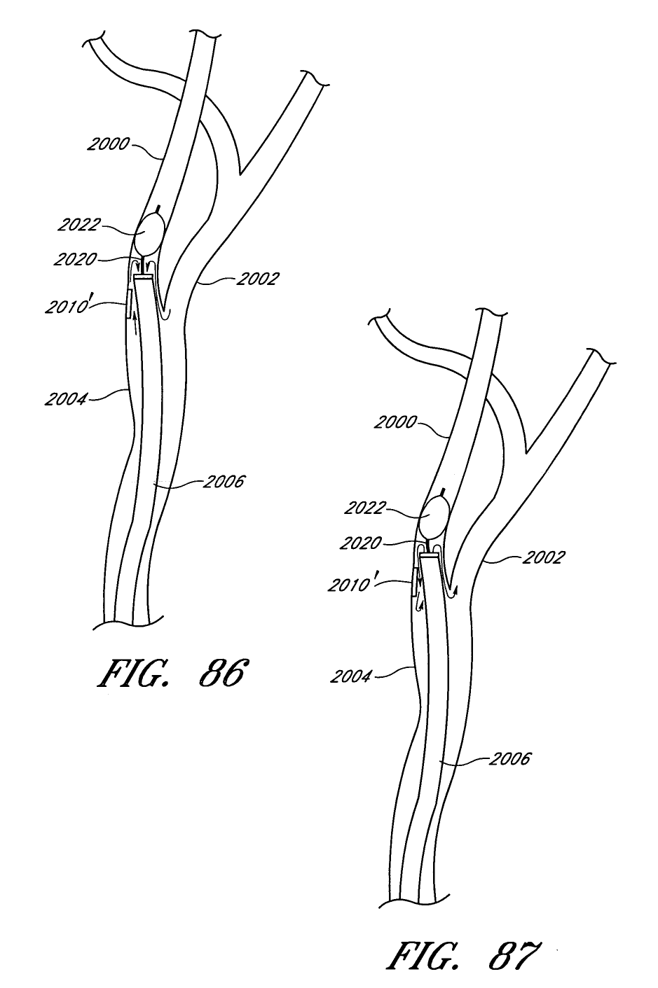

In another embodiment illustrated in FIG. 79A, a third occlusive device 2030 is used to occlude the external carotid artery 2002. Once the main catheter 2006 is in place and the common carotid artery 2004 is occluded, inner catheters 2020, 2032 are delivered to both the internal 2000 and external 2002 carotid artery branches and occluded. Following therapy and aspiration of the internal carotid artery 2000, the aspiration catheter is moved in a proximal direction, and delivered over the inner catheter 2020 into the external carotid artery branch 2002. Aspiration is then performed in that branch to remove any particles or debris that may have been moved into the external carotid artery 2002. The three occlusive devices can be alternately deactivated and reactivated as described above, to ensure the desired clearance of the working area. When aspiration is complete, the occlusive devices are deactivated, and the main 2006, aspiration, and inner catheters 2020, 2032 are removed from the patient.

Should it be desired that a separate irrigation catheter be used to provide irrigation fluid, an irrigation catheter can be delivered to the site of the occlusion following therapy and removal of the therapy catheter. The irrigation catheter is delivered through the main catheter and over the inner catheter. Irrigation fluid is provided through the irrigation catheter, while aspiration is provided through the main catheter.

FIG. 79B illustrates an embodiment like that of FIG. 79A, except that a filter 2022′ is provided in the internal carotid artery distal to the occlusion instead of a balloon.

C. Single Balloon System

In another embodiment illustrated in FIG. 80, only a single occlusive device is used. As described above, a main catheter 2006, with or without a distal occlusive device, is introduced into the patient's vasculature through an incision in the femoral artery in the groin of the patient or through direct access to the arteries in the neck. The main catheter 2006 is guided through the vasculature until it reaches the common carotid artery 2004, where it can remain in place throughout the procedure.

Once the main catheter 2006 is in place proximal to the occlusion 2010, an inner catheter or guidewire 2020 having an occlusive device 2022 at its distal end is delivered through the main catheter 2006 into the internal carotid artery 2000 and past the site of the occlusion 2010. Alternatively, a detachable occlusive device can be deployed at the site distal to the occlusion, and the delivery device removed. In this example, the occlusive device 2022 is an inflatable balloon. The balloon is inflated to occlude the internal carotid artery 2000 at a site distal to the occlusion 2010. As noted before, it should be understood that the occlusion within the artery can be in a discrete location or diffused within the vessel. Therefore, although placement of the distal occlusive device is said to be distal to the occlusion to be treated, portions of the diffuse occlusion may remain distal to the occlusive device.

A therapy catheter, not shown, is then delivered. Again, the therapy catheter can be any of a number of devices, including a balloon catheter used to perform angioplasty, a catheter which delivers a stent, a catheter for delivering enzymes, radiation, chemicals, or drugs to dissolve and treat the occlusion, an atherectomy device, a thrombectomy device, a rheolitic device, a device which creates a venturi effect within the artery, or a laser or ultrasound device used to ablate the occlusion.

Once the desired therapy is performed, the therapy catheter is withdrawn from the patient's body and an intermediate catheter 2026 is delivered through the main catheter 2006. A single operator type catheter may be used in which only a portion of the catheter rides over the guidewire, which is inserted into a separate guidewire lumen (as illustrated in FIG. 81). Alternatively, an over-the-wire type catheter can be used. The intermediate catheter 2026 is delivered into the internal carotid artery 2000 to a location just proximal to the occlusive device 2022. Preferably, in order to maximize the effectiveness of the aspiration or irrigation, the catheter 2026 is positioned less than two centimeters from the proximal end of the occlusive device 2022 at some point during aspiration. Delivering the intermediate catheter 2026 in such close proximity to the occlusion device 2022 will allow the creation of a turbulent effect near the occlusive device during aspiration and irrigation thus aiding in the removal of the particles and debris. During aspiration, the intermediate catheter 2026 can be moved in a proximal direction, to ensure more effective aspiration of the area.

Delivery of the intermediate catheter 2026 near the occlusive device 2022 requires passing the intermediate catheter 2026 across the previously occluded vessel. In order to minimize the risk to the patient the intermediate catheter 2026 is preferably soft, small and flexible. A preferred embodiment of this invention comprises delivering a soft-tipped intermediate catheter 2026 made of a compound of a durometer 55 or less.

Once the intermediate catheter 2026 is delivered in close proximity to the occlusive device 2022, the area is first aspirated. As noted above, the intermediate catheter 2026 can be moved backward in a proximal direction during aspiration. This forward and backward movement of the intermediate catheter 2026 can be repeated as often as desired to provide effective aspiration. At some point during aspiration, the distal end of the aspiration catheter should be positioned about 2 cm or less from the proximal end of the occlusive device to ensure effective aspiration. Following aspiration, the area is irrigated by supplying a fluid, such as saline, through the intermediate catheter 2026. The irrigation fluid acts to flush any remaining particles or debris from the internal carotid 2000, to the external carotid 2002, as indicated by the arrows in FIG. 82. The steps of sequential aspiration and irrigation or flushing, can be repeated as many times as necessary to remove all of the particles and debris from the vessel.

In one embodiment, the intermediate catheter 2026 has a single lumen for delivery of aspiration pressure and irrigation fluid, such as the aspiration or irrigation catheters shown in FIGS. 68 through 77. The proximal end of the intermediate catheter 2026 is connected to a source of negative pressure (as described above) and is used to aspirate the debris and particles around the occlusive device 2022.

Following aspiration of the area, the proximal end of the intermediate catheter 2026 is connected to a source of irrigation fluid, such as saline, in order to irrigate the area near the occlusive device 2022. Preferably, the volume of fluid used to irrigate the area near the occlusive device 2022 is equal to or greater than the volume of the area between the proximal end of the distal occlusive device and the start of the internal carotid artery at the bifurcation of the common carotid artery. For example, at least 10 cubic centimeters of fluid is delivered to the area that is between the distal occlusive device and the start of the internal carotid branch, which is approximately 1-5 cubic centimeters. As a result of this irrigation, any particles or debris remaining in the internal carotid 2000 will be flushed into the external carotid 2002.

In yet another embodiment, the intermediate catheter 2026 has two lumens, one for aspiration and another for irrigation. The lumen providing aspiration is attached at its proximal end to a negative pressure source. A second lumen is attached at its proximal end to source of irrigation fluid. An advantage of this embodiment is that the particles and debris removed are in a separate lumen, eliminating the possibility that they could be flushed back into the vessel when the irrigation fluid is delivered through the same lumen as the aspiration pressure. As with the single lumen embodiment, the steps of aspirating and irrigating can be repeated as many times as necessary. Once the emboli have been flushed away, the distal occlusive device may be deactivated and removed from the patient.

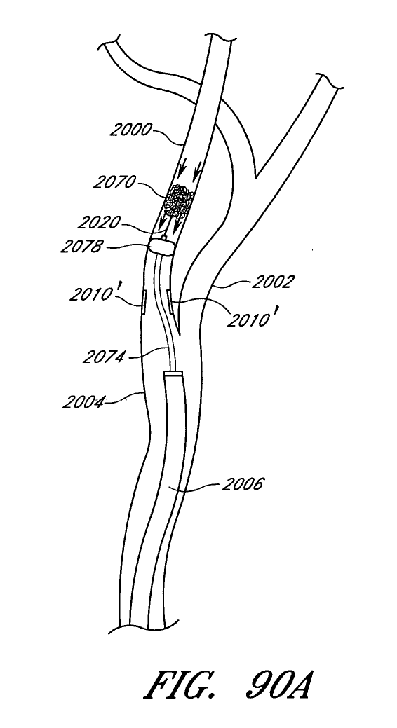

As illustrated in FIG. 83, after the occlusion 2010 has been treated, the distal end of the intermediate catheter 2026 may be advantageously placed distal to the treated occlusion, i.e., between the treated occlusion 2010′ and the occlusive device 2022. This facilitates more thorough flushing of the region between the occlusive device 2022 and the treated occlusion 2010′, and around the treated occlusion generally, so that any particles and debris remaining after therapy can be more effectively removed as the aspiration fluid (i.e., blood from the common carotid) passes across the treated occlusion 2010′.

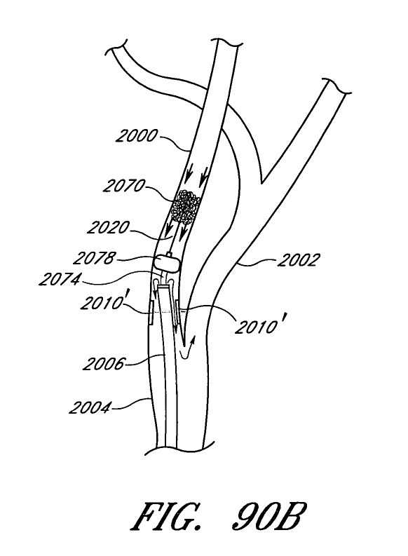

Flushing of the region in and around the treated occlusion 2010′ may be accomplished in a number of ways. For example, as illustrated in FIG. 84, the intermediate catheter 2026 may be used as an aspiration catheter. Alternatively, as illustrated in FIG. 85, the intermediate catheter 2026 may be used as an irrigation catheter, in which particles and debris are flushed towards the point where the common carotid 2004 intersects the internal carotid 2000, and towards the external carotid 2002. Upon reaching the external carotid 2002, particles, debris, and emboli are flushed down the external carotid with anatomical blood flow. The therapy catheter 2048 of FIG. 85A may also be used for irrigation following therapy, in which saline solution is pumped through the annulus between the therapy catheter 2048 and the guidewire 2020. The therapy catheter 2048 advantageously comprises a therapy device (such as a balloon for balloon angioplasty, a stent, an atherectomy device for cutting away plaque, or a rheolitic catheter) for use after the occlusive device 2022 is deployed.

The steps illustrated by FIGS. 84 and 85 may be performed sequentially, i.e., the intermediate catheter 2026 may first be used as an aspiration catheter and then as an irrigation catheter. In FIGS. 84 and 85 (or 85A), the distal end of the catheter 2026 (or therapy catheter 2048) is preferably positioned beyond the treated occlusion 2010′ to more efficiently remove emboli from the treated region. In general, aspiration is performed as close to the occlusive device 2022 as possible. The intermediate catheter 2026 (like the therapy catheter 2048) is advantageously separate from the guidewire 2020 and slidable on it, so that the catheter 2026 may be properly positioned by the user.

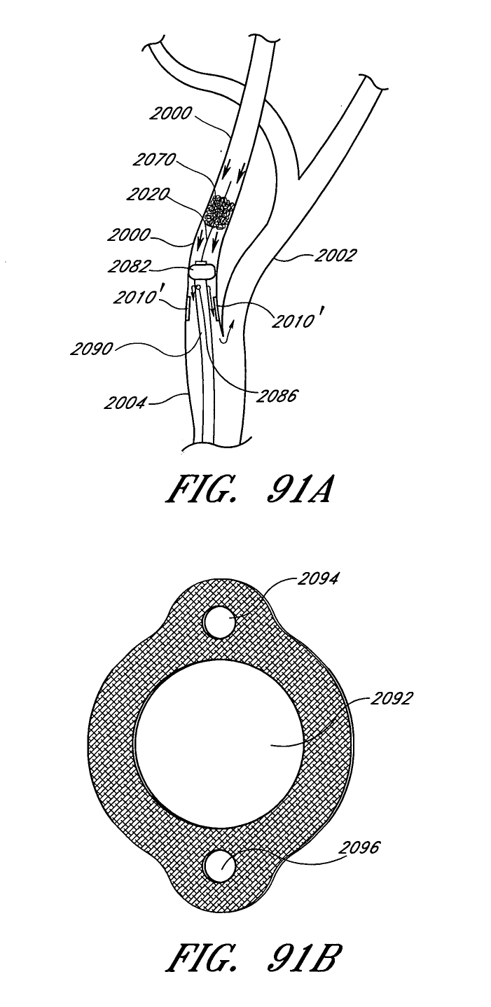

The main (outer) catheter 2006 itself may be used for aspiration and irrigation of the region in and around the treated occlusion 2010′, as illustrated in FIGS. 86 and 87, respectively. The main catheter 2006 has a radial extent that permits the therapy catheter 2048 and the intermediate catheter 2026 to pass through the main catheter 2006. In FIG. 87, the distal end of the main catheter 2006 is positioned distal to the treated occlusion 2010′, and blood containing emboli or other particles is aspirated away into the main catheter and removed from the patient. In FIG. 87, the main catheter 2006 is used to flush the region in and around the treated occlusion 2010′ by ejecting, for example, saline solution which then transports particles and debris away from the internal carotid 2000 and down the external carotid 2002. Either of the treatment methods illustrated by FIGS. 86 and 87 may be used to flush away particles, or both may be used sequentially, e.g., the main catheter 2006 may be used for aspiration and then for flushing. (However, the presently preferred methods utilize the main catheter 2006 only for irrigation.)

Other combinations of the methods illustrated by FIGS. 84-87 can be utilized. For example, the intermediate catheter 2026 of FIG. 84 may be used for aspiration, followed by flushing with the main catheter 2006 (as illustrated in FIG. 87). Also, the main catheter 2006 may be used for aspiration (as illustrated in FIG. 86), followed by flushing with the intermediate catheter 2026 (as illustrated in FIG. 85). In general, the best results are obtained by first aspirating (thereby removing particles from the patient) and then flushing, and the aspirating and flushing steps may be repeated as necessary. The same catheter can be used for aspiration and then irrigation, but in this case, this catheter should be removed from the patient and cleaned after aspiration to remove emboli from it. Irrigation then removes any emboli remaining in the patient. (However, if the main catheter 2006 is used for both aspiration and irrigation, this cleaning step should be foregone since the main catheter 2006 should be the last catheter to be removed from the patient.) It is preferred, however, to use a separate aspiration catheter and irrigate through the main catheter 2006.

Aspiration and irrigation may be performed simultaneously by having two catheters deployed at the same time and irrigating through one (e.g., the therapy catheter 2048 or the intermediate catheter 2026) and aspirating through another (e.g., the main catheter 2006). In this case, the main catheter 2006 may be deployed to a location near the intersection of the internal carotid 2000 and the external carotid 2002. Alternatively, aspiration may be performed through the intermediate catheter 2026 while irrigating through the main catheter 2006, in which the distal ends of the intermediate catheter 2026 and the main catheter 2006 are preferably positioned distal and proximal, respectively, to the treated lesion 2010′.

As shown in FIG. 88, flushing may also be performed in a blood vessel (not necessarily a bifurcated vessel as illustrated herein), using an occlusive device 2022′ that passes saline solution (or another suitable flushing solution). In this embodiment, the saline solution may be advantageously passed through a lumen in the guidewire 2020 and into the occlusive device 2022′. The occlusive device 2022′ has at least one fluid flow opening and is preferably microporous on its proximal end, having a plurality of holes 2050 (e.g., 1610-1650) that are preferably less than 1000 microns in diameter and more preferably between 50 and 100 microns in diameter. The holes may be formed in the occlusive device 2022′ by laser drilling, for example. As saline solution passes through the occlusive device 2022′ and into the internal carotid 2000, emboli, particulates, and other debris are flushed past the treated occlusion 2010′ and down the external carotid 2002. During irrigation, the fluid flow may be maintained with a pressurized syringe located outside the patient. However, while therapy is being performed on the occlusion 2010, the fluid flow may be advantageously reduced to avoid overpressurizing that segment of the internal carotid artery 2000 between the occlusion device 2022′ and the occlusion 2010 (pressures should be kept less than 50 psi). Thus, the saline solution is used for inflating the occlusive device 2022′ as well as for irrigating emboli from the internal carotid 2000 down the external carotid 2002. The irrigation method of FIG. 88 may be augmented by aspirating the region in and around the treated occlusion 2010′ through a catheter, e.g., through the intermediate catheter 2026 (as in FIG. 84) or the main catheter 2006 (as in FIG. 86).

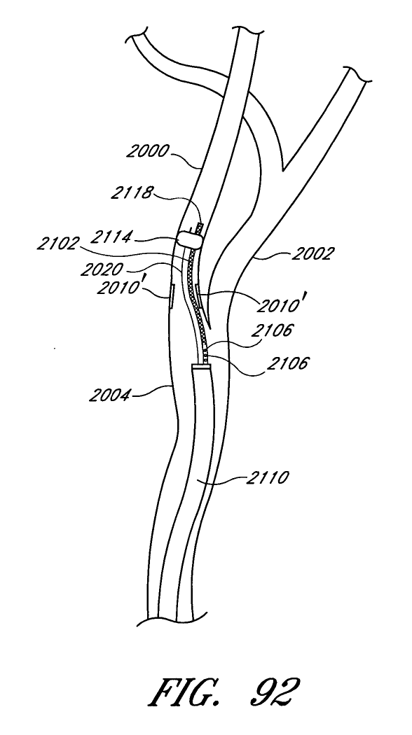

Another irrigation device and method is disclosed in FIG. 89, in which one or more holes 2060 in the guidewire 2020 are located distal to the treated lesion 2010′ and proximal to the occlusive device 2022. (For example, 1, 2, or 3 holes of dimensions 0.050″.times.0.002-0.003″ maybe used, or 10 holes of dimensions 0.003″.times.0.003″, to provide a flow such that the pressure inside the vessel does not exceed 50 psi.) Irrigation fluid is pumped through the guidewire 2020 and out of the holes 2060 (which may advantageously be 50-300 microns in diameter) to flush away emboli from the treated lesion 2010′ and down the external carotid 2002. The guidewire 2020 may have a single lumen (not shown) that is in fluid communication with both the internal carotid artery 2000 (via the holes 2060) and the occlusive device 2022, in which case the irrigation fluid and the fluid used to inflate the occlusive device 2022 are the same. Alternatively, the guidewire 2020 may have dedicated lumens (not shown) for irrigation and inflation. The irrigation method of FIG. 89 may be augmented by aspirating the region in and around the treated occlusion 2010′ through a catheter, e.g., through the intermediate catheter 2026 (as in FIG. 84) or the main catheter 2006 (as in FIG. 86).

Instead of pumping irrigation fluid through the holes 2060 as shown in FIG. 89, a larger slot (not shown) of dimensions 0.005″.times.0.100-0.200″ may be cut into the guidewire 2020 and then covered with a braid (not shown) that extends 0.010-0.030″ beyond the edges of the slot. As irrigation fluid is passed through the guidewire 2020, the braid expands, permitting the irrigation fluid to pass out of the slot and into the internal carotid 2000. Instead of using a braid, this slot may alternatively be covered with a plastic sheath (not shown) having a plurality of slits or pores (not shown) which are in fluid communication with the slot. Ten pores having a diameter of 50-100 microns may advantageously be used.

Irrigation rates for the methods disclosed herein are preferably between 0.1 cc/sec and 3 cc/sec, more preferably between 0.5 and 1.5 cc/sec, and still more preferably about 1 cc/sec. Aspiration rates are preferably between 0.5 and 5 cc/sec, and more preferably between 0.5 and 1.1 cc/sec. The fluid pressure used to generate the irrigation and aspiration rates may be pulsed on and off to better flush away emboli. For example, fluid pressure may be alternately applied for 5 seconds (in the form a pulse) and then turned off for 2-3 seconds. In general, fluid is irrigated (or aspirated) through a lumen in a catheter, with the lumen being in fluid communication with a fluid flow opening at a distal end portion of the catheter.

The single balloon methods disclosed herein may additionally comprise inflating a balloon on the main catheter 2006 within the common portion of the vessel to occlude the common portion.

D. Alternate Dual Balloon System

Under certain circumstances, use of a second occlusive device is desired, as illustrated in FIG. 82. The second occlusive device 2032 is positioned on the distal end of the main catheter 2006 and acts to occlude the main carotid artery 2004. A second occlusive device 2032 may be desired where the physician is concerned about crossing the occlusion 2010 in the internal carotid artery 2000 with the inner catheter 2020 or where there is another occlusion 2028 in the external carotid artery 2002 resulting in decreased flow through the external carotid 2002.

Once the main catheter 2006 is delivered to the common carotid artery 2004, the occlusive device 2032 is activated. The activation of the occlusive device 2032 will have the effect of occluding the common carotid artery 2004 thereby cutting off the blood flow to both the internal carotid 2000 and the external carotid 2002 arteries.

Next, an inner catheter 2020 with an occlusive device 2022 is delivered distal to the occlusion 2010 in the internal carotid artery 2000 and activated, thus isolating the occlusion 2010 between the two occlusive devices 2032, 2022. This is followed by therapy on the occlusion 2010 as described above. Sequential aspiration and irrigation are then performed as described above.

The main advantage of using two occlusive devices is that when the internal carotid artery 2000 is irrigated, a back pressure is created in the chamber defined by the proximal occlusive device 2032 and the distal occlusive device 2022. This back pressure will force the fluid, particles and debris from the internal 2000 and common 2004 carotid arteries through the external carotid artery 2002.

E. Alternate Triple Balloon System

In some cases, a triple balloon system is used. This system is especially advantageous in those patients where occlusion of the common carotid artery results in blood from collateral vessels flowing from the external carotid artery to the internal carotid artery. The direction of blood flow in a particular patient can be determined through angiography.

In this system (not shown), following activation of the occlusive device in the common carotid artery, but before crossing the occlusion with an inner catheter, a first inner catheter with an occlusive device is delivered to the external carotid artery and the occlusive device activated. This prevents flow from collateral blood vessels moving from the external to the internal carotid artery. Next, a second inner catheter is delivered to the internal carotid artery past the site of the occlusion and the occlusive device activated to occlude the internal carotid artery. Alternatively, the first inner catheter can be positioned within the internal carotid artery and the occlusive device activated, followed by delivery of the second inner catheter to the external carotid artery and activation of the occlusive device. In either case, the occlusion is completely isolated between the three occlusive devices. This is followed by therapy on the occlusion, aspiration, and irrigation if desired, as described above.

F. Use of Occlusive Devices in Combination with Perfusion

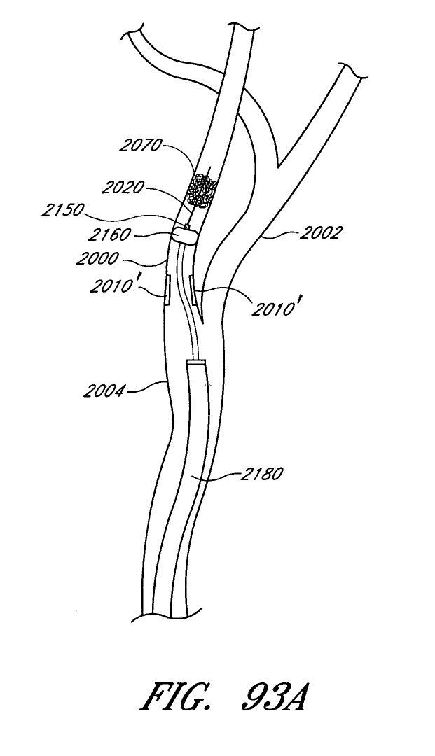

For some applications, it may be desirable to provide for the perfusion of blood while medical procedures are performed on a blood vessel such as the internal carotid artery. Several such embodiments are now discussed in connection with FIGS. 90-93. Perfusion may be necessary for those patients who can not tolerate an excessive reduction of blood flow to their brain. One exemplary method is illustrated in FIGS. 90A and 90B. FIG. 90A shows a guidewire 2020 to which an expandable member 2070 is attached. In its undeployed state, the expandable member 2070 has a relatively narrow profile so that the member 2070 may be delivered through a blood vessel. The expandable member 2070 may be, for example, a mesh- or filter-like device which, when deployed, permits the perfusion of blood but is capable of entraining emboli. After positioning the (undeployed) expandable member 2070 distal to the lesion 2010 within the internal carotid artery 2000 to be treated, the member 2070 may be deployed so that it expands to fill the internal carotid artery. The expandable member 2070 may be deployed by any one of a number of techniques, for example, the distal end of the member 2070 may be attached to a pull wire (not shown) that passes through the guidewire 2020 such that when the pull wire is retracted, the member 2070 expands to fill the internal carotid artery 2000. (Conversely, in this example, the member 2070 may be returned to its undeployed position when the pull wire moves in the distal direction.) Alternatively, the member 2070 may be self-expanding and deployed by retracting a low profile sheath (not shown) that surrounds the expandable member.

After the expandable member 2070 is deployed, therapy may be performed on the lesion 2010, resulting in a treated lesion 2010′. To this end, a therapy catheter (not shown in FIGS. 90A and 90B) may be deployed over the guidewire 2020, and any one of a number of therapy operations performed, such as balloon angioplasty, deploying a stent, or the application of drug therapy. The therapy catheter may then be withdrawn from the patient. Emboli that are created as a result of the therapy are blocked by the expandable member 2070 when these emboli are flushed distally by anatomical blood flow, so that the emboli are prevented from traveling downstream towards the brain, for example. However, the expandable member 2070 still permits the perfusion of blood, so that the health of tissue supported by the internal carotid artery 2000 is not jeopardized.

Following removal of the therapy catheter, an intermediate catheter 2074, to which an occlusive device 2078 (such as an occlusion balloon) is attached, is directed through the common carotid 2004 (by passing the catheter 2074 over the guidewire 2020 and through a guide or main catheter 2006) and positioned such that the occlusive device 2078 is distal to the treated lesion 2010′ and proximal to (and preferably adjacent proximal to) the expandable member 2070. The occlusive device 2078 may then be deployed to occlude the internal carotid artery 2000, thereby preventing anatomical blood flow. Emboli that have been entrained within the expandable member 2070 may then be advantageously aspirated away by applying suction through the intermediate catheter 2074, such that blood distal to the expandable member 2070 passes through the expandable member, entraining emboli in the process. The aspirated emboli and blood pass through the intermediate catheter 2074 and are directed out of the patient. If the expandable member 2070 is clogged, aspiration will nevertheless draw emboli from the expandable member by drawing blood from the proximal side of the expandable member.

To remove any emboli that may be remaining in and around the treated lesion 2010′, the main catheter 2006 may be brought distal to the treated lesion, as illustrated in FIG. 90B. With the occlusive device 2078 still deployed, irrigation fluid is ejected from an opening near the distal end of the main catheter 2006, such that emboli are carried towards the intersection of the internal carotid 2000 and the external carotid 2002, whereupon the emboli are flushed down the external carotid 2002 by anatomical blood flow. The expandable member 2070 may be retracted, preferably while aspirating through the intermediate catheter 2074, so that any emboli that are dislodged during the retraction of the expandable member are removed from the patient. The expandable member 2070, the intermediate catheter 2074, and the main catheter 2004 may then be removed from the patient.

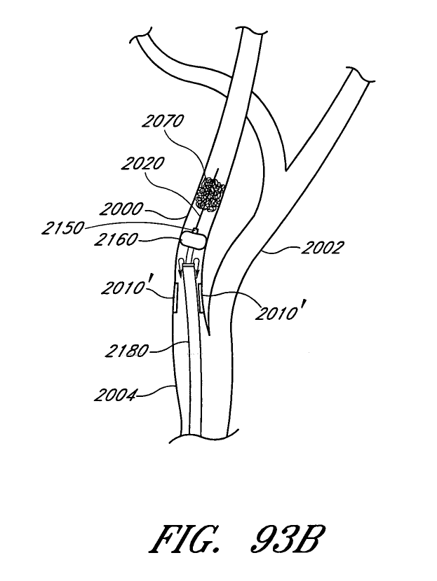

Another embodiment in which an occlusive device is combined with perfusion is illustrated in FIGS. 91A and 91B, in which the expandable member 2070 is positioned and deployed distal to the lesion 2010 as in the embodiment of FIG. 90A. After performing therapy on the lesion 2010, a catheter such as an outer catheter or main catheter 2090 is brought distal to the treated lesion 2010′. An occlusive device 2082, such as a balloon, located on the main catheter is then deployed, occluding the vessel 2000. Aspiration is then performed through the main catheter 2090, so that blood flows through the expandable member 2070 and into the main catheter, thereby removing emboli trapped in the expandable member, which may have been produced as a result of therapy. If the expandable member 2070 is clogged, aspiration will nevertheless draw emboli from the expandable member by drawing blood from the proximal side of the expandable member. Irrigation fluid may be advantageously ejected from one or more openings 2086 in the main catheter 2090 distal to the treated lesion 2010′, so that emboli remaining in and around the treated lesion are flushed towards the intersection of the internal carotid 400 and the external carotid 2002, whereupon they are flushed down the external carotid by anatomical blood flow. The aspiration and irrigation steps may be performed sequentially or simultaneously. While the expandable member 2070 is retracted, aspiration is preferably performed to remove any emboli that are dislodged during the process of retracting the member 2070.

FIG. 91B shows the cross section of the main catheter 2090, illustrating that the main catheter 2090 of FIG. 91A has at least three lumens—a lumen 2092 for passing the guidewire 2020 and the therapy catheter (not shown in FIG. 91A), a lumen 2094 for inflating the occlusive device 2082, and a lumen 2096 for delivering irrigation fluid to one or more openings 2086 distal of the treated lesion 2010′.

Yet another embodiment in which an occlusive device is combined with perfusion is illustrated in FIG. 92, in which a hypotube2102 having a plurality of holes 2106 is passed through an outer or main catheter 2110. In the embodiment illustrated in FIG. 92, an occlusive device such as an occlusion balloon 2114 is used rather than the expandable member 2070, and the hypotube 2102 extends beyond the occlusion balloon 2114 in the distal direction to provide perfusion while therapy is performed on a lesion 2010.

After positioning the (uninflated) occlusion balloon 2114 distal of the lesion 2010 and positioning an opening 2118 of the hypotube 2102 distal of the occlusion balloon 2114, the occlusion balloon is inflated so that the occlusion balloon occludes the vessel 2000 while contacting the hypotube 2102. (The holes 2106 in the hypotube 2102 are preferably located at least 1-2 mm proximal of the lesion to reduce the risk of emboli entering the holes and traveling distal of the occlusion balloon 2114.) Therapy is then performed on the lesion 2010, while the inflated balloon 2114 blocks emboli (produced as a result of the therapy) from traveling downstream. However, blood may still pass through the holes 2106 in the hypotube 2102 and exit the opening 2118 in the hypotube, so that perfusion of blood is allowed. After the therapy is complete and the therapy catheter is removed, a fluid port of the catheter 2110 may be advantageously positioned distal to the treated occlusion 2010′ (and proximal to the balloon 2114) and irrigation fluid delivered distal to the treated lesion 2010′, so that fluid flows across the treated occlusion in a distal to proximal direction. The irrigation fluid carries away emboli towards the junction of the internal carotid 2000 and the external carotid 2002, whereupon the emboli are flushed down the external carotid by anatomical blood flow. As an alternative (not shown) to using a hypotube dedicated for perfusion, holes may be introduced into the guidewire, with the guidewire having a lumen that extends through the occlusion balloon leading to an opening distal of the balloon. The holes 2106 in the hypotube 2102 or in the guidewire 2020 may advantageously have diameters of 0.005″ or larger, or slits of dimensions 0.005″.times.0.005″. Several such hole (or slits) are preferably used to create a flow of blood of between 8 and 50 cc/min.

The perfusion illustrated in FIG. 92 is passive, since the blood passes through the hypotube 2102 on its own. In the case of active perfusion, a pump such as a syringe pump (not shown) may be attached to the proximal end of a hypotube that does not have holes. Alternatively, a guidewire such as guidewire 2020 may be used in which the guidewire passes through the occlusive device 2022—such a guidewire has a lumen therein having an opening which is distal to the balloon 2114. The lumen could have a diameter between 0.010″ and 0.025″, and more preferably a diameter of about 0.018″. A preferred pump rate is between 8 and 40 cc/min.

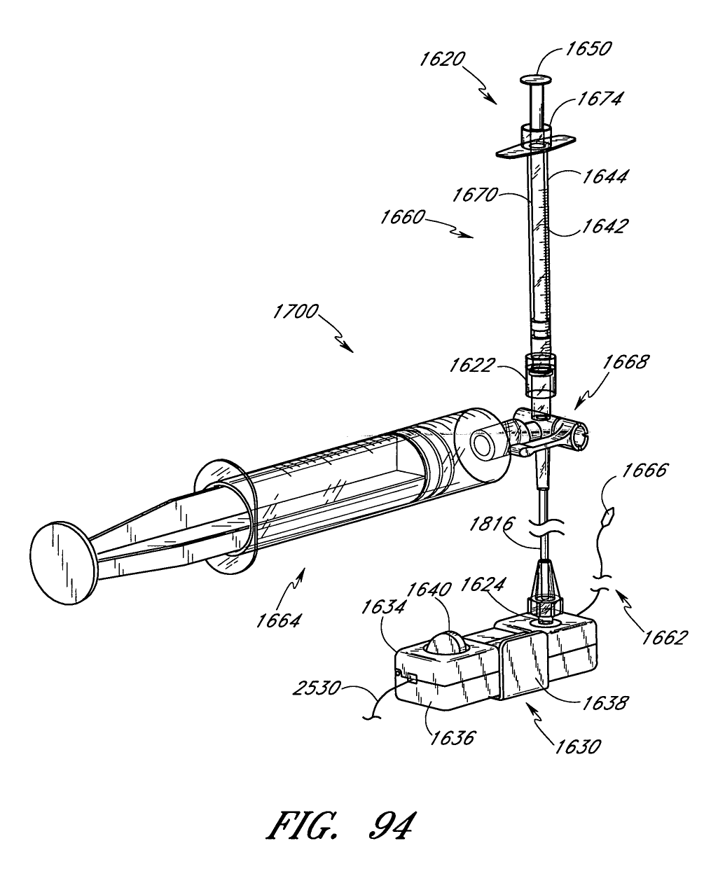

Another embodiment that combines features of occlusion with perfusion is illustrated in FIGS. 93A and 93B. The guidewire 2020 is brought through the vessel 2000 until the expandable member 2070 is located distal to the lesion 2010 to be treated. After deploying the expandable member 2070, therapy is performed on the lesion 2010, e.g., using a therapy catheter 2150 to which an angioplasty balloon 2160 is attached. During and after performing therapy on the lesion 2010, the expandable member 2070 collects emboli that may be produced as a result of the therapy, as blood travels from one side of the expandable member to the other in a proximal to distal direction. After performing therapy (e.g., after deploying the angioplasty balloon 2160), the angioplasty balloon 2160 is deflated and the therapy catheter 2150 is moved over the guidewire 2020 in the distal direction such that the angioplasty balloon 2160 is distal of the treated lesion 2010′. The angioplasty balloon 2160 is then reinflated so that the vessel 2000 is occluded. With the vessel 2000 occluded, a catheter 2180 such as an outer or main catheter is then positioned such that a fluid port of the catheter 2180 is distal of the treated lesion 2010′. As illustrated in FIG. 93B, irrigation fluid is ejected from the catheter 2180 to flush away any emboli that may remain in and around the treated lesion 2010′. The irrigation fluid and any emboli entrained within it travel in a distal to proximal direction towards the intersection of the internal carotid 2000 and the external carotid 2002, whereupon the irrigation fluid and emboli are carried down the external carotid artery 2002 by anatomical blood flow. (Alternatively, the catheter 2180 may be used to aspirate the region in and around the treated lesion 2010′ to create a flow of fluid in the proximal to distal direction.) The expandable member 2070, the therapy catheter 2150, and the catheter 2180 can then be removed from the patient. When removing the expandable member 2070 from the vessel 2000, however, care should be taken to avoid introducing any emboli into the bloodstream.

G. Accommodating Changes in Vessel Diameter

As a result of therapy being performed on a lesion, the diameter of the vessel or vessels being occluded may increase. For example, if the internal carotid artery is occluded distal to a lesion within the carotid artery, and then treatment is performed on that lesion, the diameter of the internal carotid artery may increase substantially as a result of the treatment. If the occlusive device in the internal carotid does not accommodate this increase in diameter, resulting in a break in the seal between the occlusive device and the walls of the internal carotid, the risks to the patient may be significant.

A method for avoiding this possibility involves applying an expansion force to the occlusive device beyond that which is required to seal the occlusive device to the walls of the vessel. For example, if an occlusion balloon is used as the occlusive device in the internal carotid, then the balloon may be advantageously inflated to a pressure beyond that which is required to maintain a seal in the internal carotid. As the balloon begins to be inflated, it will expand both axially and radially. The balloon continues to expand radially until it mates with the walls of the vessel, at which point further expansion of the balloon in the radial direction is hindered by the tendency of the vessel to resist enlargement. Continuing to inflate the balloon at this point results in the balloon expanding preferentially in the axial direction, rather than in the radial direction. As the balloon expands in the axial direction, potential energy continues to be stored up in the balloon.

If the vessel expands (e.g., as a result of therapy being performed on it), then the potential energy stored in the balloon is harnessed in that the balloon expands in the radial direction (while correspondingly contracting somewhat in the axial direction), such that the balloon continues to make contact with the vessel during and following treatment, thereby preventing a break in the seal which could result in injury to the patient. Thus, with such a method, it is not necessary to actively adjust the pressure in the balloon as a result of treatment of a lesion, and in this sense, the seal is self-accommodating with respect to changes in vessel diameter.

The occlusive device may also comprise a self-expanding material such as nitinol, for which it is possible to obtain a nearly constant level of stress over a relatively wide range of strain. For example, if an occlusive device comprising a nitinol filter-like mesh is capable of sealing a 6 mm diameter vessel, such an occlusive device may be used to occlude a vessel that is initially 5 mm in diameter, so that if the vessel expands, the perfusion-filter will also expand to maintain occlusion within the vessel.

The diameter of the internal carotid artery may increase substantially as a result of therapy performed on it. For example, a vessel that has a 4 mm diameter at the point where the occlusion balloon is located may increase to 5 mm or more as a result of therapy. Thus, in this method, the balloon may be advantageously positioned in a blood vessel such that the vessel diameter is at least 20% less than the maximum useful sealing diameter of the balloon. As the lesion is treated, the balloon will continue to seal against the walls of the vessel, even if the diameter of the vessel should expand in response to the treatment. This method can be used with a variety of expandable members other than balloons, such as braids, coils, ribs, ribbon-like structures, slotted tubes, and filter-like meshes, which may be partially or completely covered with a membrane or another covering to provide a seal with the vessel.

H. Inflation Apparatus

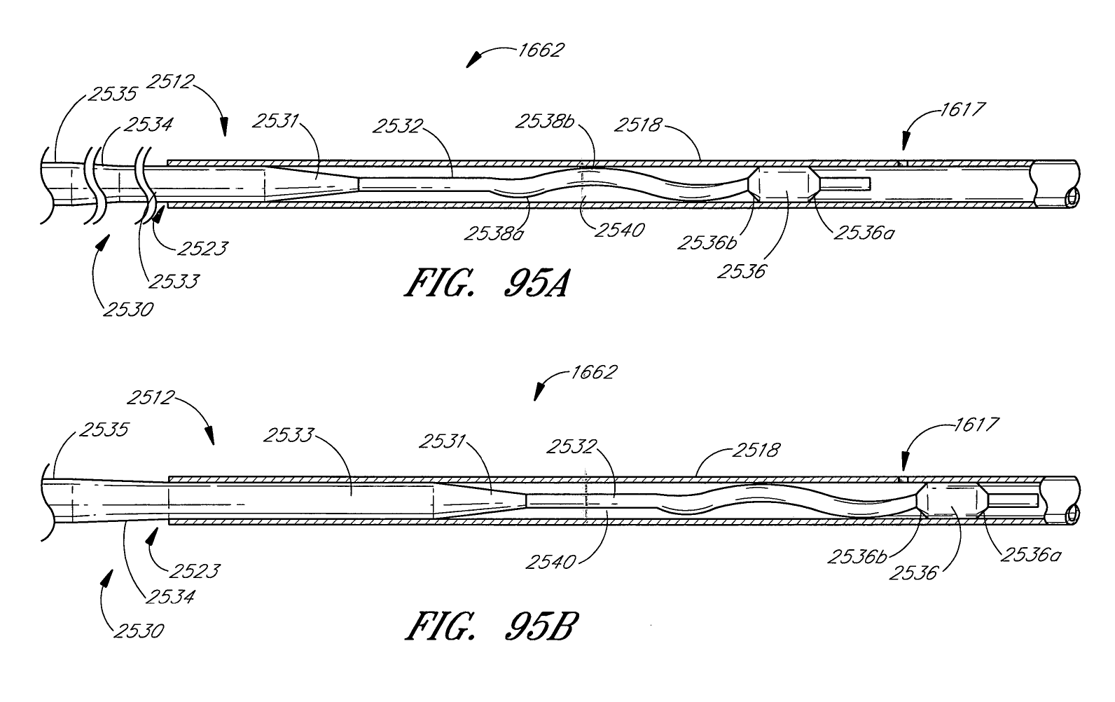

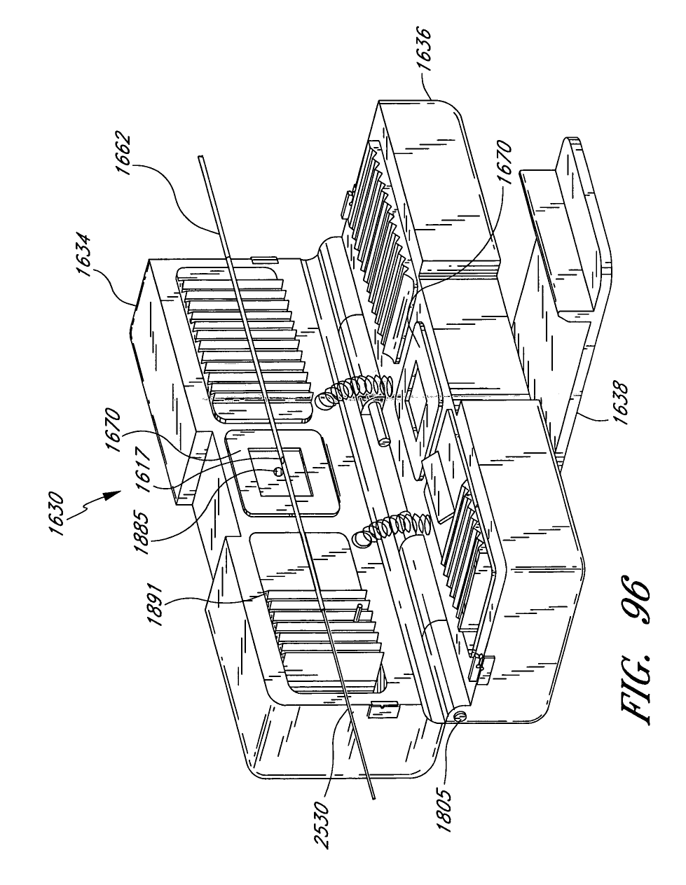

A preferred embodiment of a low volume or inflation syringe 60 in a syringe assembly 100 for inflating an occlusion balloon in accordance with the present invention is shown in FIG. 94. Also shown in FIG. 94 is an illustrative connection of the assembly 1700 to an occlusion balloon guidewire catheter 1662 (such as guidewire 2020) utilizing an inflation adapter 1630. The syringe assembly 1700, comprising the inflation syringe 1660 and a larger capacity or reservoir syringe 1664, is attached via tubing 1816 to the inflation adapter 1630 within which a sealing member 2530 (see FIGS. 95A and 95B) and the balloon catheter 1662 are engaged during use. Alternatively, other devices that control the flow of inflation fluid, such as flow controllers, may be used.

The sealing member 2530, described in more detail below in connection with FIGS. 95A and 95B, is inserted into an open proximal end of the catheter 1662. The syringe 1660 is used to inject inflation fluid through the adapter 1630 and inflation port 1617 into a lumen of the catheter 1662, and into a balloon 1666 (such as balloon 2022). The inflation adapter 1630, described in more detail below in connection with FIG. 96, is used to open and close the sealing member 2530 to permit the inflation or deflation of the balloon 1666 mounted on the distal end of the catheter 1662. However, it will be emphasized that other types of adapters, valves, and/or sealing members can be employed with the inflation syringe and/or syringe assembly of the present inflation, in order to achieve rapid and accurate inflation/deflation of medical balloons or other nonballoon medical devices. Therefore, although illustrated in connection with a low volume occlusion balloon 1666, other types of balloons and nonballoon devices may be utilized.

If the balloon 1666 is mounted on the distal end of the catheter 1662, the syringe 1660 and/or syringe assembly 1700 is preferably connected at the proximal end of the catheter 1662. Prior to use of the syringe 1660 to inflate the balloon 1666 to the proper size for the vascular segment to be treated, the distal end of the catheter 1662 and the balloon 1666 are first “primed” or evacuated. The reservoir syringe 1664 of the assembly 1700 may be used for the evacuation. Access to the vascular site is through a port in the patient obtained, for example, using an introducer (not shown). A preferred system and method for accomplishing the occlusion balloon inflation is described below.

The inflation syringe 1660 may be provided with a stop mechanism 1620 for limiting both the intake of fluid into the syringe and the delivery of fluid from the syringe. The syringe 1660 has an elongate cylinder 1644 and plunger arrangement 1650 which provide for greater displacement or travel by the plunger along the cylinder length than is necessary to expel a relatively small amount of inflation fluid. Thus, with the stop mechanism 1620, the clinician is provided with an enhanced sense of whether the fluid in the syringe 1660 has been delivered to the balloon, which helps compensate for lack of precision by the clinician. The stop mechanism 1620 may be mounted on the syringe 1660 during production, or as separate components that can be retro-fit onto an existing supply of syringes.

Referring to FIGS. 94, 95A, 95B, and 96, the catheter 1662 has the sealing member 2530 inserted into its proximal end and has a side-access inflation port 1617, shown in greater detail in FIGS. 95A and 95B. The inflation port 1617, proximal end of the catheter 1662 and distal end of the sealing member 2530 are positioned within the inflation adapter 1630 (see FIG. 96) to which a syringe assembly 1700 in accordance with the present invention has been operably coupled. The inflation syringe 1660 is coupled via an injection cap 1622 at its distal end to a valve 1668 that also connects the large capacity syringe 1664 and a short tube segment 1816. The tube segment 1816 is adapted to connect to a fitting or male luer member 1624 of the inflation adapter 1630. Thus, the sealing member 2530 is engaged by the adapter 1630 to allow use of the low volume syringe 1660 of the syringe assembly 1700 to inflate the balloon 1666 at the end of the catheter 1662.

The catheter 1662 (depicted in FIGS. 95A and 95B) has a proximal end 2512, and a distal end (not shown in FIGS. 95A and 95B) to which is mounted the inflatable balloon 1666. A central lumen 2540 extends within a tubular body 2518 between the proximal and distal ends. An opening 2523 to lumen 2540 is present at the proximal end 2512 of catheter 1662. The inflation port 1617 in fluid communication with lumen 2540 is provided on tubular body 2518.

The sealing member 2530 is inserted into lumen 2540 through central lumen opening 2523. Sealing member 2530 has a first region 2535 which has an outer diameter substantially the same as the outer diameter of the proximal end 2512 of the catheter tubular body. Region 2535 has a taper 2534, reducing in diameter to a second region 2533 which has an outer diameter less than the inner diameter of lumen 2540. In one embodiment, region 2533 tapers over length 2531 to form a plug mandrel wire 2532. As a consequence, region 2533 and plug mandrel wire 2532 are slidably insertable into the proximal opening 2523 of catheter 1662 and may move within lumen 2540. In one preferred embodiment, region 2535 has an outer diameter of about 0.013 inches, region 2533 has an outer diameter of about 0.008 inches, and plug mandrel wire 2532 has a diameter of about 0.006 inches, with region 2533 and plug mandrel wire 2532 being inserted into a catheter having a central lumen 2540 with an inner diameter of about 0.009 inches.

The length of sealing member region 2535 extending proximally of catheter 1662 may vary in length depending upon the intended use environment. For example, where catheter 1662 is to be used as a guide for other catheters in an “over-the-wire” embodiment, it is preferred that the total length of catheter 1662 and sealing member region 2535 be about 300 centimeters. Alternately, where catheter 1662 is to be used in a single operator or rapid exchange embodiment, it is preferred that the total length of catheter 1662 and region 2535 be about 190 centimeters. Accordingly, with a known catheter length and use environment, an appropriate length for region 2535 may be chosen.

Regions 2535 and 2533 and plug mandrel wire 2532 may all be made out of metals such as stainless steel. Alternatively, combinations of materials may be used as well. For example, in some applications it may be desirable to manufacture regions 2535 and 2533 out of stainless steel, while manufacturing plug mandrel wire 2532 out of nitinol. Furthermore, the various sealing member regions may be made from a single metal wire strand coined at various points to achieve the desired dimensional tolerances, or multiple segments may be joined together to form sealing member 2530.

Where multiple segments are joined, region 2535, region 2533, and plug mandrel wire 2532 are attached to one another by any suitable means of bonding metal to metal, such as crimping, soldering, brazing, adhesives and the like. In one preferred embodiment, cyanoacrylate adhesives are used to adhere these various parts of sealing member 2530 to one another.

As illustrated in FIGS. 95A and 95B, the outer diameter of sealing member region 2533 is less than the inner diameter of lumen 2540, such that region 2533 is slidably insertable into lumen 2540. In addition, the outer diameters of the tapered portions 2531 and wire 2532 are also small enough such that they too are slidably insertable in lumen 2540. However, the outer diameter of region 2535 is greater than the inner diameter 2540, and thus only a small portion of tapered portion 2534 of sealing member 2530 between region 2535 and region 2533 is insertable into lumen 2540 through opening 2523. Advantageously, this provides for a snug interference fit when sealing member 2530 is fully inserted into catheter 1662. This interference fit provides a frictional force which counteracts the tendency of the pressurized fluids and internal wire flexing in the catheter to push sealing member 2530 out of opening 2523.

As illustrated in FIGS. 95A and 95B, sealing member 2530 has movement-force increasing structure which increases the force required to move sealing member 2530 within lumen 2540. The movement-force increasing structure consists of waves 2538a and 2538b formed in wire 2532 near its distal end. Waves 2538a and 2538b contact the inner surface of lumen 2540, thereby increasing the frictional force which must be overcome to move wire 2532 within lumen 2540. In one preferred embodiment, wire 2532 is made of nitinol and has an outer diameter of about 0.006 inches, and is inserted into a nitinol catheter which has an inner lumen 2540 with a diameter of about 0.009 inches. In one embodiment, waves are formed on wire 2532 for 3 cycles with an amplitude of about 0.019 inches to increase the valve-opening movement force. Alternatively, by increasing the length over which wire 2532 contacts the inner wall of the tubular body 2518, the frictional forces may be increased.

A lumen sealer portion 2536 is coaxially and fixedly mounted on wire 2532. Sealer portion 2536 forms a fluid tight seal with the outer diameter of wire 2532 and the inner diameter of lumen 2540, such that fluid introduced into lumen 2540 through the inflation port 1617 is prevented from flowing past sealer portion 2536 when sealer portion 2536 is inserted into lumen 2540 distally of the inflation port 1617. Sealer portion 2536 forms the fluid tight seal by firmly contacting the entire inner circumference of a section of lumen 2540 along a substantial portion of the length of sealer portion 2536.

As shown in FIG. 95A, sealer portion 2536 is positioned proximally of the inflation port 1617, so that an unrestricted fluid passageway exists between inflation port 1617 and the inflatable balloon at the distal end of catheter 1662, which is like a valve “open” position. In this position, region 2533 is shown partially withdrawn from opening 2523. Referring to FIG. 95B, sealer portion 2536 is positioned distally of inflation port 1617, so that fluid flow between inflation port 1617 and the inflatable balloon 1666 at the distal end of catheter 1662 are substantially blocked, which is like a valve “closed” position.

Catheter 1662 is changed from the valve open position to the valve closed position by the movement of sealing member 2530 and its various components. Preferably, the exact length of movement needed to change catheter 1662 from the valve closed to the valve open position is built into the movement function of the adaptor used to manipulate sealing member 2530 thereby opening and closing the catheter valve. In this regard, it is preferred that catheter 1662 be used with an adaptor such as adaptor 1630, which provides for such controlled precise movement.

The “stroke-length”, or overall movement in one dimension, of sealing member 2530 required to open or close the valve may be varied depending upon the catheter requirements. When relying upon the inflation adaptor to control movement, however, it is important that the movement of the controlling elements of the adaptor be coordinated with those of sealing member 2530.

Referring to FIGS. 94 and 96, the inflation adapter 1630 comprises a housing having two halves 1634, 1636 preferably formed of metal, medical grade polycarbonate, or the like. In one embodiment, the halves 1634, 1636 are attached by hinges 1805 to be separated or joined in a clam shell manner. A locking clip 1638 secures the halves while the adapter 1630 is in use. A groove within the housing has a width to accept the proximal end of the catheter 1662 having the sealing member 2530. The male luer member 1624 (FIG. 94), or other suitable connector, extrudes from a top of the housing to provide an inflation passageway. Seals 1670 are provided within the housing and around the internal segment 1885 of the inflation pathway to conduct the pressurized fluid provided by the syringe 1660 attached to the male luer member 1624.

An actuator 1640, shown in FIG. 94 at the top of the adapter housing, controls a cam which operates sliding panels 1891 (FIG. 96) contained in the housing. Preferably, the catheter 1662 is positioned within the housing with the sealing member 2530 in the closed position (FIG. 95B), such that the side inflation port 1617 is located in the sealed inflation area 1885 of the housing. An adjacent proximal portion of the catheter 1662 extends outside the housing (and into the patient), and a proximal portion of the sealing member 2530 extends out of the other side of the housing. The locking clip 1638 is then secured and then the syringe 1660 may be attached. The actuator 1640 is moved from a first position to a second position, such that the sliding panels 1891 within the housing cause the sealing member 2530 to be in an open position to allow fluid flow through the inflation port 1617 (FIG. 95A). “Closing” the sealing member 2530 is accomplished by moving the actuator 1640 from the second position back to the first position (FIG. 95B), such that the balloon inflation is maintained.

While the foregoing detailed description has described several embodiments of the apparatus and methods of the present invention, it is to be understood that the above description is illustrative only and not limiting of the disclosed invention. It will be appreciated that the specific dimensions of the various catheters and guidewires can differ from those described above, and that the methods described can be used within any biological conduit within the body and remain within the scope of the present invention. Thus, the invention is to be limited only by the claims which follow.

What is claimed is:

1. A method for treating an occlusion in an internal carotid artery, comprising:
delivering a first catheter carrying a first occlusive device to a location in a common carotid artery proximal to the occlusion;
expanding the first occlusive device within the common carotid artery;
delivering a second occlusive device through a lumen in the first catheter to a location in an external carotid artery;
expanding the second occlusive device within the external carotid artery;
delivering an expandable filter through the lumen in the first catheter to a location distal to the occlusion in the internal carotid artery, the filter being carried by a guidewire;
expanding the filter distal to the occlusion in the internal carotid artery;
delivering a therapy device to the occlusion in the internal carotid artery over the guidewire; and
treating the occlusion with the therapy device.

2. The method of claim 1, further comprising aspirating emboli generated from treating the occlusion.

3. The method of claim 2, wherein emboli are aspirated through the lumen of the first catheter.

4. The method of claim 1, wherein the therapy device is an angioplasty balloon.

5. The method of claim 1, wherein the first occlusive device is a balloon.

6. The method of claim 1, wherein the second occlusive device is a balloon.

7. The method of claim 1, wherein the second occlusive device is delivered on a second catheter delivered through the lumen of the first catheter.

8. The method of claim 1, wherein the filter is self-expanding.

9. The method of claim 1, wherein the filter is expanded prior to delivering the therapy device.

10. A method for treating an occlusion in a blood vessel, comprising:
delivering a catheter carrying an occlusive device to a location proximal to the occlusion;
expanding the occlusive device proximal to the occlusion;
delivering an expandable filter through a lumen in the catheter to a location distal to the occlusion;
expanding the filter distal to the occlusion;
delivering a therapy device to the occlusion; and
treating the occlusion with the therapy device.

11. The method of claim 10, further comprising aspirating emboli generated while treating the occlusion.

12. The method of claim 10, further comprising aspirating emboli through the lumen in the catheter.

13. The method of claim 10, wherein the therapy device is an angioplasty balloon.

14. The method of claim 10, wherein the occlusive device is a balloon.

15. The method of claim 10, wherein the filter is carried by a guidewire.

16. The method of claim 15, wherein the therapy device is delivered over the guidewire.

17. A method for treating an occlusion in a branch of a bifurcated vessel comprising a common portion and two branches, comprising:
delivering a guidewire carrying an expandable filter through the common portion and into one of the branches to a location distal to the occlusion;
expanding the filter distal to the occlusion in the carotid artery;
delivering a therapy device to the occlusion over the guidewire; and
treating the occlusion with the therapy device.

18. The method of claim 17, further comprising aspirating emboli generated while treating the occlusion.

19. The method of claim 17, further comprising, prior to delivering the guidewire distal to the occlusion, delivering a catheter carrying an occlusive device into the common portion, and expanding the occlusive device in the common portion.

20. The method of claim 19, further comprising aspirating emboli through a lumen of the catheter.

21. The method of claim 20, wherein emboli are aspirated after treating the occlusion with the therapy device.

22. The method of claim 17, wherein the therapy device is an angioplasty balloon.

23. The method of claim 19, wherein the occlusive device is a balloon.

24. The method of claim 19, wherein the guidewire is delivered distal to the occlusion in an internal carotid artery, and further comprising, prior to delivering the guidewire to the occlusion, delivering a catheter carrying an occlusive device into the external carotid artery, and expanding the occlusive device in the external carotid artery.
